(19) United States
(12) Patent Application Publication
(10) Pub. No.: US 2025/0127883 A1
(43) Pub. Date: Apr. 24, 2025

(54) METHOD FOR INHIBITING ZOPHOBAS MORIO BLACK WASTING DISEASE

(71) Applicant: Rutgers, The State University of New Jersey, New Brunswick, NJ (US)

(72) Inventors: Jason KAELBER, Dunellen, NJ (US); Judit PENZES, Piscataway, NJ (US)

(21) Appl. No.: 18/920,760

(22) Filed: Oct. 18, 2024

Related U.S. Application Data
(60) Provisional application No. 63/591,484, filed on Oct. 19, 2023.

(51) Int. Cl. A61K 39/23; A61K 9/00; A61K 39/00; A61P 31/20; C12N 7/00

(52) U.S. Cl. CPC A61K 39/23; A61K 9/0019; A61P 31/20; C12N 7/00; A61K 2039/5254; A61K 2039/54; A61K 2039/552; C12N 2750/14034; C12N 2750/14051

(57) ABSTRACT

A reared colony of larval superworms (Zophobas morio) experienced the swift and unexplained death of about 90% of its population. From dead larvae, a high-abundance virus was isolated and identified as a novel densovirus, Zophobas morio black wasting virus (ZmBWV), by means of cryo-electron microscopy. Strains of this virus were sequenced and an engineered vaccine virus sequence was developed. The black wasting disease was replicated in larvae by inoculation with pathogenic strains of ZmBWV. Larvae were inoculated with a non-pathogenic strain as prophylaxis and later challenged by inoculation with a pathogenic strain. The larvae that received a non-pathogenic strain were protected from later disease and death but the larvae that received a saline solution were not. The invention provides methods and vaccine products to inhibit morbidity and mortality of ZmBWWV in darkling beetle larvae.

### CROSS-REFERENCE TO RELATES APPLICATION

This application claims the benefit of U.S. provisional application Ser. No. 63/591,484, filed 19 Oct. 2023. The entire contents of the aforementioned provisional application is hereby incorporated by reference as if fully set forth herein.

### REFERENCE TO ELECTRONIC SEQUENCE LISTING

This application contains a Sequence Listing which has been submitted electronically in .XML format and is hereby incorporated by reference in its entirety. Said .XML copy, created on Nov. 29, 2024, is name “15290-035US1.xml” and is 70,289 bytes in size. The sequence listing contained in this .XML file is part of the specification and is hereby incorporated by reference herein in its entirety.

### BACKGROUND OF THE INVENTION

### 1. Field of the Invention

The invention relates to the general field of animal health. In particular, the invention relates to a vaccine composed of a nonvirulent wild-isolate virus which is useful against a virulent virus in a beetle larvae system.

### 2. Background of the Invention

The superworm (Zophobas morio) and its close relative, the mealworm (Tenebrio molitor), are species of darkling beetles whose protein-rich larvae are dietary staples for captive reptiles, birds, and amphibians worldwide. Darkling beetles are omnivorous, and eat plants, animals, and in some cases Styrofoam™. Superworms are also under investigation as an alternative animal protein source. Commercial production of Zophobas morio larvae (superworm) is a growing industry, with applications in animal feed. Research using this species has also made recent popular press headlines for the species' potential as a next-generation approach to plastic waste management and degradation.

Yet, industrial production of the superworm is complicated by mass die-offs due to infectious disease. Reports from commercial and hobbyist growers of Z. morio that farm larval populations indicate that some growers were experiencing up to 100% mortality. The larvae are typically housed in enclosures containing many individuals because dense packing inhibits pupation. Darkling beetles are known to eat deceased or moribund members of their own species, which exacerbates the spread of infectious disease.

One disease that affects these beetles is caused by viruses of the family Parvoviridae (common name: parvoviruses) and subfamily Densovirinae (common name: densoviruses). Colonies that become infected with densoviruses can experience mortality rates of up to 100%. Insects experience symptoms such as blackening, liquefaction of the organs, and uncoordinated movement. A composition for controlling densovirus infection in live insects has not been described. Therefore, there is a need in the art for methods to prevent and treat infection by Parvoviridae.

Parvoviruses (26 PV) are small icosahedral viruses with a non-enveloped virion and linear, ssDNA genome. The family Parvoviridae possesses a remarkably diverse host spectrum of both protostome and deuterostome invertebrates as well as vertebrates. Although two out of the three subfamilies within the Parvoviridae—Densovirinae and Hamaparvovirinae—include viruses of invertebrate hosts, traditionally all invertebrate-infecting PVs are referred to as densoviruses (DVs). DVs in general display high virulence and pathogenicity, and can affect insects in their larval stage, crustaceans, mollusks, and echinoderms. Mass-reared arthropods are especially in danger of DV infection.

Members of the family Parvoviridae are united by their genome size and organization and presence of certain conserved protein domains, as well as their capsid protein structure. All parvoviruses thus far harbor a small, single-stranded DNA genome of 3.7 to 6.3 kb in length, flanked by partially double-stranded, hairpin-like DNA secondary structures, essential for parvovirus replication. The coding region of the genome contains two major expression cassettes: rep, which expresses the non-structural (NS) proteins, and cap, which expresses between one and four isoforms of the structural protein (VP). These are flanked by structured genomic termini named “inverted terminal repeats” (ITRs), which are hairpin-like and partially double-stranded. The cassette closer to the 5′ end, named rep, is capable of expressing a varied number of non-structural (NS) proteins. The cassette closer to the 3′ end, named cap, encodes for one to four structural proteins (VP). The VPs share an overlapping C-terminal region and differ from each other in N-terminal extensions.

The conserved genome organization, presence of certain domains, and capsid protein structure are characteristic of the family Parvoviridae. PV infect diverse hosts from protostome and deuterostome invertebrates to humans. Most members of the vertebrate infecting Parvovirinae and all members of the Densovirinae harbor a phospholipase A2 (PLA2) enzymatic domain in the unique N-terminal extension (VP1u) of their largest minor capsid proteins. The PLA2 domain is essential for endolysosomal egress during PV and DV intracellular trafficking.

Current approaches for avoiding this disease in commercial growing are largely dependent on rearing practices by the producer. At present, culling the entire population is the only known response to an outbreak at a facility. The disease is poorly understood, and there has been insufficient time since discovery of the causal organism for effective treatments to arise. Superworms are mainly reared for sale as pet food. Mortality of the reared insects causes loss of salable product. Avoiding this cause of mortality could therefore increase revenues for superworm sellers.

### SUMMARY OF THE INVENTION

Thus, there exists a need in the art for a method to treat and prevent Parvovirus infection in superworms and mealworms, to improve husbandry for these species.

The invention described here relates to a vaccine method useful in prevention and treatment of “Zophobas morio black wasting disease (Zmbwd)” using the NJ2-molitor virus to infect larvae.

In particular embodiments, the present invention relates to a method of inhibiting Zophobas morio black wasting disease morbidity and mortality in a darkling beetle colony in need thereof, comprising: (a) isolating a strain of densovirus from Tenebrio molitor; and (b) administering the strain of densovirus to the darkling beetle colony, wherein the strain of densovirus is non-pathogenic to the recipient beetle colony. In preferred embodiments, the strain of densovirus is SEQ ID NO:5. In some embodiments, administering is by injection, dripping, spraying or ingestion.

In certain embodiments, the invention comprises a prophylactic vaccine composition, comprising the corpses of Z. morio larvae infected with a non-pathogenic strain of densovirus selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and any combination thereof.

In certain embodiments, the invention comprises a prophylactic vaccine composition, comprising the corpses of darkling beetle larvae infected with a strain of ZmBWV that is isolated from Tenebrio molitor and does not cause mortality in the recipient species at a dose of 10⁹ genomes. Preferably, the strain is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and any combination thereof. In preferred embodiments, the darkling beetle is of the species Zophobas morio.

In certain embodiments, the invention relates to a prophylactic vaccine composition comprising purified virions of a non-pathogenic strain of densovirus and a pharmaceutically acceptable medium, wherein the sequence identity between the non-pathogenic strain of densovirus and strain NJ2-molitor (SEQ ID NO: 5) is about 96% or greater.

In certain embodiments, the invention relates to a prophylactic vaccine composition comprising: a pharmaceutically acceptable medium and a purified virions of a non-pathogenic strain of densovirus selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and any combination thereof.

In certain embodiments, the invention relates to a prophylactic vaccine composition, comprising one or more nonpathogenic densovirus strain, wherein the NS3 canonical ATG start codon is mutated, truncating the NS3 protein to fewer than 200 amino acids, instead of the length of 221 residues in the pathogenic, highly virulent strain UT-morio (SEQ ID NO: 1). Preferably, the DNA sequence identity from NJ2-molitor (SEQ ID NO: 5) is about 96% or greater. In addition, preferably the pharmaceutically acceptable medium is phosphate-buffered saline. The vaccine preferably is formulated for injection, spraying, or dripping.

### BRIEF SUMMARY OF THE DRAWINGS

Certain embodiments are illustrated by way of example, and not by way of limitation, in the figures of the accompanying drawings.

FIG. 1A is a photograph of Z. morio larvae ill with Zophobas morio black wasting disease. FIG. 1B is a photograph showing virus purification from Z. morio larvae as shown in FIG. 1A. FIG. 1C is an electron micrograph (TEM) showing empty ZmBWV particles.

FIG. 1D is an electron micrograph (TEM) showing virions. FIG. 1E presents an SDS-PAGE of heat-denatured protein.

FIG. 2A and FIG. 2B each are a surface representation (left) and cross section (right) view of the 3D structure of the full (FIG. 2A) and empty (FIG. 2B) ZmBWV virus particle. FIG. 2C presents ribbon diagrams representing the atomic model of a dimer for ZmBWV.

FIG. 3A is a graph showing the effect of injection of ZmBWV strain UT-morio at various concentrations into larvae of Z. morio on the mortality of those larvae. FIG. 3B is a graph showing the effect of dripping a suspension of strain UT-morio in phosphate-buffered saline onto the cuticle of larvae of Z. morio on the mortality of those larvae. FIG. 3C is a graph showing the effect of feeding larval corpses infected with ZmBWV strain UT-morio to larvae of Z. morio on the mortality of those larvae.

FIG. 4A is a graph showing the fraction of Z. morio larvae exhibiting symptoms of black wasting disease after various injections. 21 days prior to the challenge injection, larvae were injected with nonpathogenic strain NJ2-molitor or a sham injection containing saline alone. As a challenge injection, pathogenic ZmBWV strain UT-morio was injected, or a sham containing saline alone. Pretreatment with NJ2-molitor led to fewer symptoms after UT-morio challenge. FIG. 4B is a graph showing mortality of Z. morio larvae after various injections. 21 days prior to the challenge injection, larvae were injected with nonpathogenic strain NJ2-molitor or a sham injection containing saline alone. As a challenge injection, pathogenic ZmBWV strain UT-morio was injected, or a sham containing saline alone. Pretreatment with NJ2-molitor led to reduced mortality after UT-morio challenge.

FIG. 5 shows full genome phylogenetic calculations.

FIG. 6 is a graph presenting the virus yield (by NS1 qPCR), which varied extensively by life stage, with titers from ˜1×10⁹ gc/mL in newly hatched larvae to ˜2×10¹⁶ gc/mL for blackened carcasses.

### DETAILED DESCRIPTION OF EMBODIMENTS OF THE INVENTION

### 1. Overview and Discussion

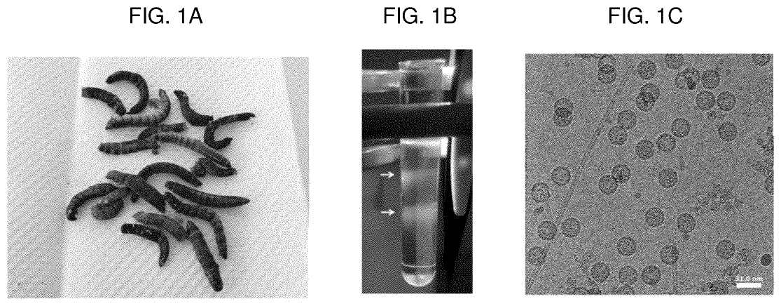

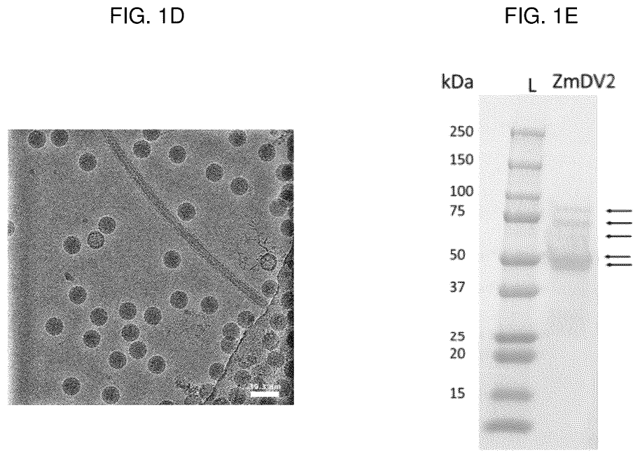

A small, commercial insect-rearing facility experienced repeated Z. morio colony collapse in 2022. At approximately 8 weeks of age and 25 mm in length, Z. morio larvae showed signs of distressed locomotion, uncoordinated wiggling, and rigor followed by death. Moribund larvae quickly blackened as their inner organs lost structure, essentially becoming liquefied. See FIG. 1A. Mortality of larvae in the colony was approximately 90%, and the surviving larvae could pupate successfully and emerge as mature beetles. The observed pathology was termed “Zophobas morio black wasting disease” at that time.

The work disclosed here has successfully identified Zophobas morio black wasting virus (ZmBWV), characterized its structure, sequence, and pathogenesis as well as identified a prophylactic mechanism that can be used to diminish it. The present invention relates to products for and methods of preventing disease in a darkling beetle population by contacting the insects with non-pathogenic densovirus. In a preferred embodiment, the invention comprises a prophylactic product that is orally available as a feed for Z. morio larvae. In a second preferred embodiment, the invention comprises a prophylactic product that is applied topically to Z. morio larvae.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “subject” refers to insect larvae of Z. morio or T. molitar, including individual larvae and populations of larvae. Preferably the larvae are captive.

As used herein, the term “subject in need” refers to any Z. morio or T. molitar larva or population of larvae that are susceptible to exposure to ZmBWV, including symptomatic and asymptomatic individuals and populations and diagnosed or undiagnosed individuals and populations.

As used herein, the term “prevent,” “prevention,” “prophylaxis” and their cognates refer to (a) complete prevention of the condition, disease, or symptom(s) thereof from occurring in a subject; (b) decreasing the likelihood of a subject contracting or developing the disease or condition; (c) inhibiting the condition, or disease or symptom thereof, such as, arresting or delaying its development in a subject; (d) causing the disease or condition to occur less frequently in a subject; (e) reducing the occurrence of the condition, disease, or symptom thereof in a population; (f) shortening the duration or reducing the severity of a disease or condition in a subject; and/or (g) relieving, alleviating or ameliorating the condition, disease, or symptom thereof, such as, for example, causing regression of the condition or disease or symptom thereof.

As used herein, the term “vaccine” refers to a pharmaceutical compound or composition that prevents a disease, condition, or a symptom thereof in a subject or a population of subjects.

As used herein, the term “densovirus” refers to any virus listed in the 9^{th} Report of the International Committee on the Taxonomy of Viruses as belonging to the subfamily Densovirinae, and additionally to any non-listed virus which has a higher sequence similarity to a virus listed as belonging to the subfamily Densovirinae than to a virus listed as belonging to another subfamily. These viruses contain a single-stranded DNA genome, an isometric capsid of 20-30 nm in diameter, and a genome length of 3,000 to 8,000 nucleotides.

As used herein, the term “ZmBWV” refers to Zophobas morio black wasting virus, a densovirus with a single-stranded DNA genome and an isometric capsid between 20 and 30 nm in diameter that infects beetles of the family Tenebrionidae (common name: darkling beetles). This term also can refer to any DNA virus with at least 90% sequence identity to SEQ ID NO:1.

### 3. Embodiments of the Invention

### A. Introduction

The causative agent of Zophobas morio black wasting disease was identified by electron microscopy and named Zophobas morio black wasting virus (ZmBWV). The etiological role of ZmBWV was confirmed by injecting ZmBWV into healthy beetles, which then exhibited the characteristic symptoms of ZmBWD. ZmBWV was detected in beetles from: Utah, Minnesota, Georgia, Maryland, New Jersey, New York, Mississippi, Ohio, Arkansas, Indiana, Pennsylvania, Oregon, Louisiana, and Florida.

Using larval corpse homogenate as a diagnostic specimen, the structure of two variants of the agent at 2.7 Å and 2 Å resolution, respectively, were identified and named Zophobas morio Black Wasting Virus (ZmBWV). This resolution was sufficient to identify the agent as a virus of subfamily Densovirinae, family Parvoviridae. Mass-reared arthropods are known to be at high risk for densovirus (DV) infection.

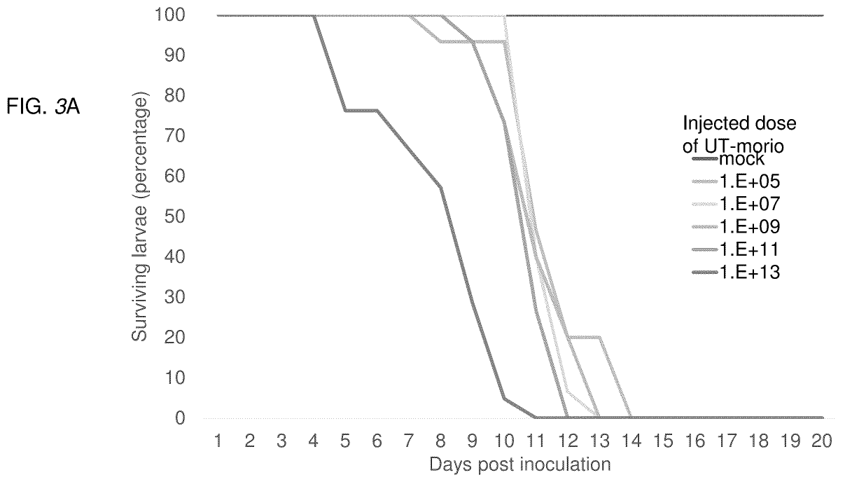

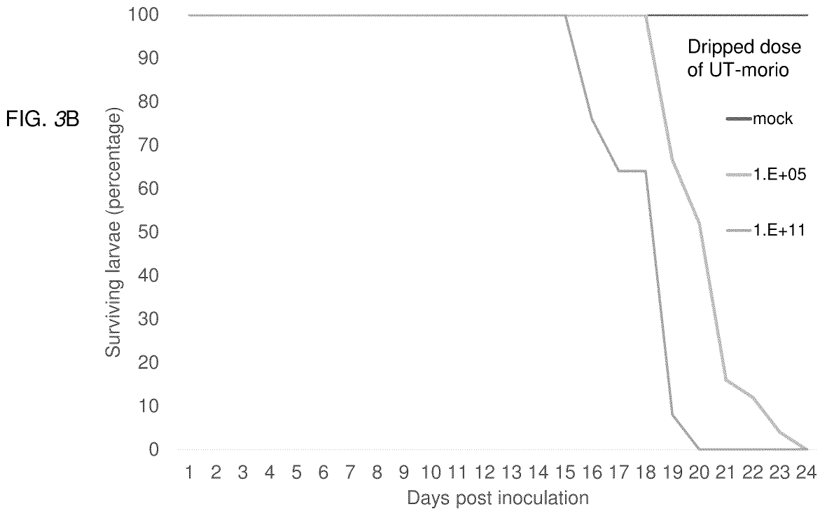

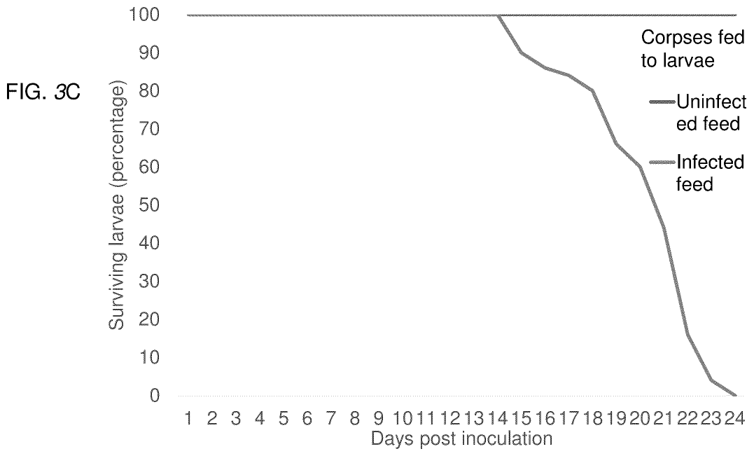

Several pieces of information indicate that this agent (ZmBWV) is the etiologic agent behind ZmBWV. First, it is uncommon for a virus to grow to such high abundance in a diseased animal unless it is the cause of that animal's disease. In this study, ZmBWV was far more abundant than all other viruses, and much more abundant than the native bacteriophages. Second, there is ample precedent for a densovirus to cause symptoms and death in farmed insects. Third, our isolation of ZmBWV from this mortality event echoes the molecular detection of ZmDV from similar outbreaks in Europe. Related DVs infecting Z. morio are believed to have a worldwide distribution. Fourth, we detected ZmBWV by quantitative polymerase chain reaction in beetles with symptoms of Zophobas morio black wasting disease from multiple states. Finally, we successfully infected unexposed larvae with purified ZmBWV virions, resulting in all individuals exhibiting the symptoms of Zophobas morio black wasting disease, which was followed by a mortality rate of 100% (See FIG. 3). Therefore, ZmBWV was treated as the presumptive cause.

Parvoviruses are notably resistant to alcohol-based sanitizers (unlike, for example, SARS-CoV-2). Therefore, ethanol is not sufficient for cleaning enclosures and other items that have come into contact with infected beetles. Bleach should be used in these cases. Similarly, by analogy to other parvoviruses (and in contrast to SARS-CoV-2), we expect the virus to last for a long time on surfaces but not to spread particularly effectively through the air. It is likely that transmission can occur through tools and clothing as well as co-housing. There is some precedent for asymptomatic carriage of DVs. Therefore, when bringing new beetles into a breeding colony, avoiding overtly symptomatic individuals might not be sufficient. As a best practice, when introgressing exogenous stock into a colony, housing part of the colony separately for a couple generations is advisable to avoid loss of the whole colony in case the new beetle(s) carry this or other pathogens. It is unknown whether immunity to ZmBWV is heritable.

Results presented here suggest that ZmBWV has reached a nation-wide epidemic status, which appears to have started four years ago. Acheta domesticus densovirus (AdDV) of the same subfamily regularly disseminates cricket rearing facilities, causing mass mortality in its wake in Europe since 1977 as well as in North America since 2009. AdDV has completely transformed the cricket rearing culture worldwide, requiring the rearing of orthopteran species that exhibit less susceptibility to the virus than the common house cricket (Acheta domesticus). Although this is the first ZmBWV outbreak to reach an epidemic scale, it is important to note that this virus also exhibits a worldwide distribution, similarly to AdDV. The broad host spectrum of ZmBWV, as well as its subclinical presence and multiple genotypes indicate that the epidemic may remain active for a long a time to come.

There are several different ZmBWV strains circulating currently in the United States, affecting both the T. molitor and the Z. morio species. Despite this, we did not observe a similar scale epidemic in T. molitor. The lack of virulence shown here by the NJ2-molitor strain in the Z. morio host suggests that the current epidemic has a distinct origin. This is also supported by the phylogenetic calculations, which suggest a single introduction event. The studies presented here, however, indicate that there is cross-protectivity between these two strains. This phenomenon can be employed to provide protection against virulent strains of ZmBWV.

As part of these studies, it became apparent that ZmBWV spreads within a colony by the oral-fecal route. The midgut has been shown to play an important role in DV infection. Lepidopteran protoambidensoviruses cross the midgut wall by transcytosis, in order to reach the true site of replication, which may be the fat bodies or the wall of the visceral trachea and hemocytes, while lepidopteran iteradensoviruses and bidnaviruses replicate exclusively in the columnar midgut cells. In both cases, however, the infection runs its course fast and larvae die within 7-10 d.p.i.

The pathogenesis of the ZmBWV virulent strain UT-morio required almost three times longer to result in mortality. Moreover, at the affected farms the newly hatched larvae were already infected, yet symptoms manifested only at the age of eight weeks. Once the larvae display the initial signs of ZmBWD, death is expected to set in less than five days.

Here, we suggest a model of pathogenesis of the virus replicating in the midgut columnar cells following uptake by contaminated feed, which eventually leads to the invasion of the fat body once the midgut wall is too damaged to fulfill its barrier function. The invasion may happen directly before the onset of symptoms. Although DV-infected caterpillars fail to pupate, ZmBWV-infected Z. morio larvae completed their entire life cycle, if they managed to pupate before the onset of symptoms. The ability of ZmBWD to cause a chronic infection in reproducing beetles may be the major reason to why the epidemic has been persistent.

ZmBWV is a member of the Blattambidensovirus genus, yet its biology differs from that of its most studied member i.e., Blattella germanica densovirus (BgDV). The minor VPs of BgDV run significantly heavier when subjected to SDS-PAGE than suggested by their predicted molecular mass due to ubiquitination. Moreover, BgDV exhibits two spliced transcripts when expressing its minor capsid proteins, which results in the protein sequence of cap1 to comprise the N-terminus of both VP1 and VP2. The ZmBWV VPs display an SDS-PAGE running profile that corresponds with their predicted molecular weights, implying that they are not subjected to the same post translational modifications. Moreover, only the protein sequence of VP1 corresponded with cap1, implying that ZmBWV only expresses one spliced VP transcript. However, we could identify a glycan at the ZmBWV surface, probably due to the N-glocalization. Although this type of post-translational modification has not been described in DVs, members of the Parvovirinae, such as adeno-associated virus (AAV), have been shown to be subjected to N-glocalization. In the case of AAV8, N-glocalization was only detectable in secreted capsids as opposed to the ones acquired by cell lysis. It is plausible that ZmBWV also acquires this modification during the process of cellular egress, which implies that it does not rely exclusively on cell lysis, leading to intact midgut cells to possibly secrete virions. This finding is in concordance with the slow initial phase of the ZmBWV pathogenesis.

This invention provides the first method and product for inhibition of ZmBWV disease in Z. morio colonies. The method involves exposure of the larvae in a colony to a nonpathogenic virus as a vaccine which reduces morbidity and mortality in the colony produced by Z. morio black wasting virus.

### B. Association of ZmBWV with Disease Endemic in the United States

Specimens of Z. morio (16 pools) and of T. molitor (8 pools) were collected from breeders who had experienced mass mortality events clinically consistent with ZmBWD (9 breeders; 11 farms), or from mail-order services (3), or from local stores (2). Using the whole-genome sequence, a diagnostic PCR targeting the NS1 gene was developed. 100% of Z. morio pools obtained from breeders with symptomatic larvae tested positive by PCR. At local stores, staff or customers reported observing the typical pathology and pools were positive from both. Mail-order Z. morio from two vendors did not exhibit symptoms; these alone were PCR-negative. These findings satisfy Koch's First Postulate. ZmBWV was detected in 11 states representing all regions of the lower 48 of the United States, and ZmBWV should therefore be considered endemic nationwide.

We did not obtain or attempt to obtain samples from other countries. The NCBI GenBank, however, already included nine metagenomic sequences of Chinese, Malaysian and European origin, derived from bird, bat and pangolin metagenomes, harboring 94-97% identity to the reference ZmBWV strain. This implies a worldwide distribution.

Breeders reported that mealworms would occasionally exhibit black wasting but economically-significant mortality was never observed. We detected a ZmBWV-like virus from all 3 mail-order T. molitor batches tested, although no signs or symptoms were observed. Likewise, 3 of 4 T. molitor pools from breeders of Z. morio were PCR-positive; two of these colonies had a few overtly symptomatic individuals and one had none. We concluded that the ZmBWV-like virus of T. molitor is of mild pathogenicity in its native host. Genomic sequences were obtained for 8 Z. morio and 5 T. molitor samples.

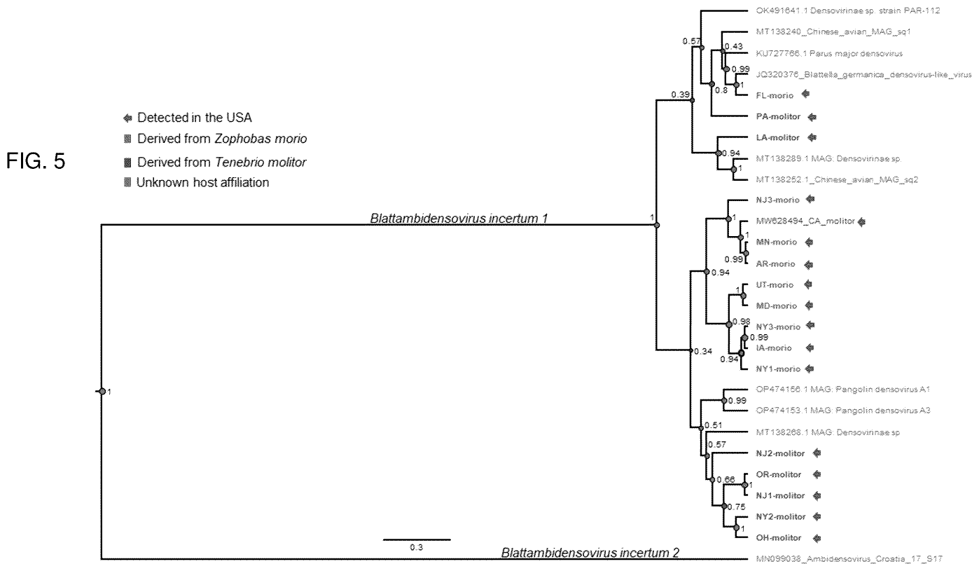

Full genome phylogenetic calculations (FIG. 5) revealed no geographic clustering, nor did sequences cluster by breeder. Paired T. molitor and Z. morio strains from the same breeding facility did not cluster with each other but rather separated by host species. Of the Z. morio contributed directly from breeders (as opposed to being purchased on the open market), only one pool was believed by the breeder to be ZmBWV-negative at time of shipment. These larvae did sicken and die a few days later, and ZmBWV was detected. Interestingly, the genome of that strain (but not of any other strain isolated from Z. morio) clustered with T. molitor strains (FIG. 5). It is possible that this is a low-pathogenicity strain. All pathogenic ZmBWV strains clustered together.

### C. Original Mass Mortality Report of Z. morio

A densovirus associated with mass mortality in Z. morio larvae at a small-sized insect rearing facility in the western United States was investigated. Z. morio larvae approximately two months of age and about 25 mm in length were observed to show signs of distressed locomotion, uncoordinated wiggling, and rigor followed by death. The deceased larvae quickly blackened as their inner organs lost structure, essentially becoming liquefied (see FIG. 1A). Within a week from the first detection of signs, 90% of the larvae died.

Interestingly, an outbreak of similar pathology occurred in the Z. morio larvae stock of the Moscow Zoo in 2015, with PCR detection revealing a DV as the causative agent. The partial genome of this DV has been deposited to the GenBank under the name Zophobas morio densovirus (ZmDV). ZmDV was first described in Hungary in 2014, with similar symptoms. The DV sequence, revealed in both studies, disclosed 97% nucleotide sequence identity with Blattella germanica densovirus-like virus (BgDVLV), a member of the Densovirinae genus Blattambidensovirus. BgDVLV has a genome of over 5.1 kb in length (the length of the 77 yet-unsequenced genome termini are unknown), and it utilizes an ambisense gene expression strategy.

### D. PCR and DNA Quantification

The primer pair 5′-GACAGCGGATACTATGTGTCA-3′ (SEQ ID NO: 11) and 5′-AATTTCAAGAGGAAGTCTTTG-3′ (SEQ ID NO:12) was designed to target an approximately 300 nucleotide long, highly conserved region of the NS2 gene. The primers were designed to be capable of amplifying the respective genome region of all members within the Blattambidensovirus incertum1 species, hence this PCR system could be used for diagnostic purposes, i.e., detecting the presence of ZmBWV DNA in the sample. Amplification was executed in a 25 μL final reaction volume, including 2 μL of purified DNA target, 0.5 μL of both primers in 50 pmol concentration, 0.5 μL dNTP mix with 8 pmol of each nucleotide, and 0.1 μL of DreamTaq™ DNA polymerase enzyme (Thermo Fisher™) PCR reactions were executed under a program of 5 minutes denaturation at 95° C. followed by 35 cycles of 30 seconds denaturation at 95° C., 30 seconds annealing at 50° C., and 1 minute of elongation at 72° C. The final elongation step was 5 minutes long at 72° C.

Quantitation of the viral DNA was carried out by real-time PCR amplification (qPCR), using an Applied Biosystems QuantStudio 5 instrument. This quantitation process was executed to estimate viral titers in the samples and in vaccine formulations. A 300-bp-long target sequence was amplified by the primers mentioned above. For dsDNA quantitation the SYBR™ Green PCR Master Mix (Applied Biosystems™) was used, with an amplification program of 5-minute denaturation at 95° C. followed by 45 cycles of 30 second denaturation at 95° C., 15 seconds annealing at 55° C., and 30 seconds of elongation at 72° C. Results were analyzed by the QuantStudio™ Pro software (Applied Biosystems™). This was used exclusively for diagnostic purposes. Material from PCR-positive larvae was subjected to whole-genome sequencing, either without further purification or after purification of virions by sucrose gradient centrifugation.

### E. Vaccine Densoviruses

The vaccines according to embodiments of this invention include the following strains of ZmBWV, deemed non-virulent,
FL-morio, GenBank accession number OR026173,
PA-molitor, GenBank accession number OR026186,
LA-molitor, GenBank accession number OR026175,
NJ2-molitor, GenBank accession number OR026179,
OR-molitor, GenBank accession number OR026185,
NJ1-molitor, GenBank accession number OR026178,
NY3-molitor, GenBank accession number OR026182,
OH-molitor, GenBank accession number OR026184,
   which preferably are provided in a pharmaceutical formulation suitable for administration to Z. morio larvae. All potential vaccine strains possess an N-terminally truncated version of the NS3 protein, due to a single nucleotide polymorphism altering the first ATG start codon to ATT (encodes Ile) of the reading frame (nt position 314, the 22^{nd} codon of the complete open reading frame). The truncated protein product has a predicted length of 220 residues, as opposed to the 371 amino acids in case of the virulent strains. In the closely related Blattella germanica densovirus, NS3 is a small protein of 31 kDa, which is localized diffusely in the host cell nucleus during viral replication (Kopalinskaya). Apart from the naturally occurring non-virulent ZmBWV strains introducing the same mutation to the genome of the virulent strains may result in the creation of additional vaccine strains.

### F. Vaccine Compositions and Uses

Vaccine compositions according to the invention can take several forms. According to certain embodiments, the composition is produced for administration to beetle larvae per os. In such cases, the composition comprises beetle larvae that have been infected by any of injection, spraying, dipping, or administration as a feed, and then killed. Killing preferably is accomplished by freezing, suffocation by carbon dioxide exposure, and the like, as is convenient. The killed larvae may be administered as is, without any further processing, or may be crushed, sliced, freeze-dried, air-dried, or the like for suitable packaging for sale. Vaccine compositions for oral administration are added to an enclosure containing larvae to be treated with the vaccine. The larvae eat the infected material and are thereby infected with the vaccine densovirus. See FIG. 3C.

In alternative embodiments, vaccine compositions include solutions of densovirus material in a pharmaceutically acceptable carrier such as phosphate-buffered saline. The carrier also can contain optional additives such as antibacterial agents, pH modifiers, antifungals, non-ionic surfactants, stabilizing agents, and the like. Such solutions preferably are formulated as a spray or dip solution, or alternatively for injection. See FIG. 3B. Alternatively, the infectious vaccine strain may be mixed in to a gel-like formula of pectin or agarose, similar to the hydrating formulation sold under the name “cricket water”.

The dose of the vaccine for an individual larva is about 10⁷ genome copies (gc) to about 10¹¹ gc; preferably about 10⁸ gc to about 10¹⁰ gc; and most preferably about 10⁹ gc for administration by injection. The dose of the vaccine for administration by feeding for an individual larva is about 10¹⁰ gc to about 10¹⁵ gc; preferably about 10¹¹ gc to about 10¹³ gc; and most preferably about 10¹² gc. For administration to an individual by spraying or dipping, the larvae is subjected to the virus suspension in PBS by direct application on the cuticle.

For a population of individual larvae (such as 1000 individuals) the dosage of the vaccine is about 10¹¹ gc to about 10¹³ gc; preferably about 1×100¹² gc to about 5×10¹²; and most preferably about 1×100¹² gc to about 2×10¹² per 10 ml of injectable suspension, dosing each larva by ˜10 μl. For spraying, about 10 mL to about 20 mL per thousand individuals of a solution of 10¹⁴ gc in buffer, e.g. phosphate buffered saline, at a concentration of about 10¹³ gc/ml to about 10¹⁴ gc/ml; preferable about 10¹³ gc/ml to about 5×10¹³ gc/ml; and most preferably about 10¹³ gc/ml is administered to the population.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. Samples and Animal Husbandry

Specimens of Z. morio were shipped directly to our laboratory from 11 farms, located in the states of Arkansas, Florida, Georgia, Maryland, Mississippi, Ohio, New Jersey, New York, and Utah, with two farms located in the state of New York. T. molitor samples were obtained from the farms in Ohio, New Jersey and one New York farm. Z. morio larvae also were purchased from two facilities, located in the states of Oregon and Pennsylvania. T. molitor samples were obtained the same way from both facilities, as well as from Louisiana. Samples of both T. molitor and Z. morio, bred in the states of Indiana and Minnesota, were purchased at local pet stores. Dubia roaches and buffalo beetles originated from one of the farms located in New York.

Z. morio larvae were housed in plastic insect breeder boxes at room temperature and were given a piece of fresh carrot every second day. Oatmeal bran was used as bedding. Experimentally inoculated larvae were housed in single use plastic dessert cups, also on oat bran bedding. Animals were euthanized prior to processing in either dry ice or by freezing at −80° C. Strain NJ2-molitor was isolated from Tenebrio molitor. It is not pathogenic in Zophobas morio.

### B. Purification of Viral Particles

Deceased Z. morio larvae were subjected to tissue homogenization in 1× phosphate-buffered saline (PBS), followed by three cycles of freeze-thaws. Following this, the homogenate was combined with an equal volume of 1×TN™ pH8 (50 mM Tris pH8, 100 mM NaCl, 0.2% Triton X-100, 2 mM MgCl₂) and the debris were removed by centrifugation at 3700×g at 4° C. for 15 minute intervals until the supernatant was sufficiently cleared. The supernatant was mixed with 1×TNET pH8 (50 mM Tris pH8, 100 mM NaCl, 0.2% Triton X-100, 1 mM EDTA) in a 1:3 ratio and concentrated on a cushion of 20% sucrose in TNET using a type 45 Ti rotor for 3 h at 4° C. at 42,300 rpm on a Beckman Coulter S class ultracentrifuge. The pellet was resuspended in 1 mL of 1×TN™ pH8 and, following overnight incubation, purified on a 5 to 60% sucrose step gradient for 3 hours at 4° C. at 35,000 rpm, using the same instrument with a SW 41 Ti swinging bucket preparative ultracentrifuge rotor. Both visible bands were aspired by a single needle puncture and a 10-mL volume syringe. The purified fractions were dialyzed against 1×PBS in order to remove the sucrose.

### C. Preparation of CryoEM Grids and Plunge Freezing

Quantifoil™ R1.2/1.3 300 mesh grids were glow discharged and coated with a 2.62-nm-thick carbon film. The film was fabricated by electron-beam deposition on cleaved mica using a Leica™ EM ACE600 instrument and floated onto a surface of ultrapure water through which the discharged grids were lifted. In case of the ZmBWV NJ2-molitor strain UltrAfoil™ R1.2/1.3 300 grids were used. Samples were plunged-frozen into liquid ethane using a Vitrobot Mark IV (FEI) at 100% humidity and ambient temperature. The grids were clipped into autoloader grids and imaged using a Talos Arctica™ transmission electron microscope (TEM) (Thermo Fisher™), equipped with a Gatan K2 direct electron detector, operated in low dose mode.

D. Identification of a Parvovirus by cryoEM

Z. morio carcasses received from the Utah facility were homogenized and subjected to sucrose gradient fractionation. Two bands, at the interfaces between the 20% and 25% sucrose steps and between the 35 and 40% sucrose steps, were obtained. Both contained isometric viral particles of approximately 26 nm in diameter; the more-buoyant particles were hollow but the less-buoyant particles contained dense intracapsid material corresponding to the viral nucleic acid. This virus is referred to here as “Zophobas morio black wasting virus” (ZmBWV).

3D maps of the empty capsids and full virions at 2.7 and 2.9 Å resolution, respectively, were obtained. Both were T=1 icosahedral capsids with a single capsid protein per asymmetric unit with a jelly roll core. The Cu backbone was manually traced by building polyalanine chains of 423 and 425 residues in length per asymmetric unit, respectively. Qualitatively, we assessed that the backbone model exhibited a prototypical parvovirus fold. Quantitatively, we queried all extant protein structures from the Protein Data Bank (PDB) using DALI (Holm 2019) to detect homologs, and obtained three hits with a z-score over 20, namely: Galleria mellonella densovirus capsid (GmDV) (1DNV, z-score of 29.9), Acheta domesticus densovirus (AdDV) capsid (4MGU, z-score of 29.4) and Bombyx mori densovirus (BmDV) capsid (3POS, z-score of 23). All of these are members of the Densovirinae subfamily of the Parvoviridae. Automated, background-knowledge-agnostic tools were used to reproduce this determination. We automatically traced, and assigned sequence to, the cryoEM density using ModelAngelo (Jamali^{25b}. DALI search of the longest detected chain revealed the same result pattern, albeit with lower z-scores: GmDV, AdDV, and BmDV followed by PVs of other subfamilies. Meanwhile, a HHblits search of the sequence profile from ModelAngelo against UniProt detected similarity to Blatella germanica densovirus (BgDV) with a p-value of 10⁻⁷⁷. Sequencing later confirmed that these results were completely correct: ZmBWV is a densovirus whose closest known relative is BgDV. We conclude that cryo-EM can be used for sequencing-free discovery of novel viral species from clinical or environmental samples.

### E. Collection of High-Resolution Data and 3D Reconstruction

Selected cryoEM grids were subjected to high resolution data collection, using electron microscopy (operated at 200 kV, with a 10-s-long exposure and a total dose of 43.16 e−/A297, using a frame length 0.2 seconds). Movie frames were recorded in counting mode using the Serial EM suite (Mastronarde 2018) at a sampling of 1.038 Å/pixel (ZmBWV Utah) and 0.658 Å/pixel (ZmBWV Nj-molitor). The collected movies were aligned by the MotionCorr2 application with dose-weighting. The cisTEM software was used for single-particle image reconstruction to obtain an initial model. High resolution single particle reconstruction was carried out by Relion 4.0 (Zivanov) and CryoSparc (Punjani). Micrograph quality was assessed by CTF estimation using a box size of 512. The subset of micrographs with the best CTF fit values were included in further processing.

Particles were automatically boxed by the particle selection subroutine of CisTEM, at a threshold value of 2.0 or by the blob picking subroutine of CryoSparc live, during on-the-fly processing. Boxed particles were subjected to 2D classification, imposing icosahedral symmetry at 35 classes. Particles of classes, which failed to display a clear 2D-class average of the icosahedral particle, were eliminated from the reconstruction, resulting in the incorporation of 42,219 and 2265 particles in the full and empty capsid reconstructions, respectively. Ab initio model generation was carried out in iterations, imposing icosahedral symmetry. The obtained startup volume was subjected to automatic refinement under icosahedral constraints and underwent iterations until reaching a stabile resolution. To improve the resolution, corrections for higher-order aberrations, beam tilt, trefoil and anisotropic magnification were implemented, as well as astigmatism was fitted for each micrograph and CTF parameters were fitted per particle. In case of using the Relion suite, particles were subjected to Bayesian polishing, obtaining the training parameters based on 10 000 particles. The automatic high-resolution refinement was repeated in the presence of a mask with a soft edge. The maps were subjected to sharpening or to the post processing subroutine to obtain the final reconstructions. The final maps were achieved by sharpening at a post cutoff B-factor of 20. The resolution of each reconstructed map was calculated based on a Fourier shell correlation (FSC) of 0.143. The obtained cryoEM maps were visualized in Coot to model the backbone of one subunit. Visualization was carried out by UCSF Chimera. Data collection parameters and refinement statistics are shown in Table 1, below.

TABLE 1
Data Collection and Statistics.

| | | UT-morio | | | OR-molitor* |
|---|---|---|---|---|---|
| Processing and | UT-morio | empty | NJ2-molitor | OR-molitor* | empty |
| Refinement Parameters | virion | capsid | virion | virion | capsid |
| Total number of micrographs | 4515 | 9312 | 1944 | 7635 | |
| Reconstruction software | cisTEM | cisTEM, | CryoSPARC | cisTEM | cisTEM, |
| | Relion 4.0 | Relion 4.0 | | | Relion 4.0 |
| Defocus range (μm) | 0.8-2.7 | 0.6-1.5 | 0.8-4.32 | 0.6-1.2 | |
| Electron dose (e⁻/Å²) | 43 | 29 | 32 | 33 | |
| Frames/micrograph | 50 | 40 | 30 | 30 | |
| Pixel size (Å/pixel) | 1.011 | 1.011 | 0.63 | 1.011 | 0.627 |
| Starting number of particles | 93965 | 133622 | 156329 | 151035 | |
| Particles used for final map | 42219 | 41960 | 15964 | 11874 | 47770 |
| Resolution of final map (Å) | 2.7 | 2.9 | 2.0 | 3.3 | 2.7 |
| PDB ID | 8T9C | 8T9E | 8T9X | 8TAE | 8TA7 |
| Residue range (VP2) | 165-593 | 171-593 | 165-593 | 165-593 | 171-593 |
| Map correlation coefficient | 0.7766 | 0.8148 | 0.8396 | 0.8277 | 0.8084 |
| RMSD (root-mean-square | 0.003 | 0.005 | 0.008 | 0.003 | 0.003 |
| deviation) [bonds] (Å) | | | | | |
| RMSD [angles] (Å) | 0.537 | 0.623 | 0.745 | 0.533 | 0.588 |
| All-atom clash score | 7.97 | 7.86 | 3.99 | 7.99 | 8.58 |
| Favored (%) | 97.9 | 96.88 | 97.94 | 97.78 | 97.17 |
| Allowed (%) | 2.10 | 2.88 | 2.06 | 2.22 | 2.83 |
| Outliers (%) | 0.02 | 0.25 | 0 | 0 | 0 |
| Rotamer outliers (%) | 0 | 0 | 1.3 | 0 | 0 |
| C-β deviations | 0 | 0 | 0 | 0 | 0 |

The cryoEM maps were visualized in Coot to model the backbone of one subunit. Visualization was carried out by UCSF Chimera. The density was modeled by Coot and ISOLDE (Croll) and the obtained models were refined in PHENIX.

The cryo-EM structure provided important details about the viral genome, which allowed determination of the most economical sequencing option to obtain its complete sequence. A PCR-based diagnostic tool was developed and used to investigate the extent and pathogenesis of the ZmBWV epidemic. This diagnostic pipeline, however, has no limitations to invertebrate animals only; it can be implemented in the identification and monitoring of human-infecting viral pathogens with similar efficiency.

### F. Microscale X-Ray Computed Tomography

Z. morio larvae were fixed in 4% paraformaldehyde. To preserve the healthy larvae in a straightened-out position, they were first placed to 4° C. for 15 minutes and only in this immobile state were they placed to the paraformaldehyde solution. Specimens were washed in 1×PBS three times then stained in aqueous 1% iodine and 2.5% potassium iodide to enhance internal features. Staining was carried out for 24 hours. Following staining, the sample was washed in a 0.9% sodium chloride solution. The specimens were scanned in a Skyscan 1272 instrument, at the voltage of 60 kV with a 166 μm source current. Images were collected at the pixel size of 4.5 μm, as the frame average of three per a 4° rotation step. The completed 2D scan images were reconstructed by the Skyscan Nrecon software and rendered in the Amira software (Thermo Fisher™)

### Example 2. Assessment of the Pathology of ZmBWV Infection

ZmBWV (strain “Utah,” from the index case) was administered to healthy, 4-week-old, PCR-negative Z. morio larvae. Three forms of administration were contrasted: injection of purified virus into the fat body, dripping purified virus suspension onto larval cuticles, or (exploiting the naturally cannibalistic tendency of Z. morio) feeding blackened carcasses of infected insects (ZmBWV titer in carcasses at about 10¹⁶ gc/mL). See FIG. 3. By all three methods, larvae exhibited the same pathology as described above in natural infection, viz.: distressed locomotion, loss of coordination in wiggling, blackening, and eventually liquefaction and death. This confirms Koch's Third Postulate.

Time to symptoms and death varied with route of administration and with titer. Direct injection was the most lethal; the LD₅₀ by injection is below 10⁵ gc but is between 10⁹ and 10¹³ gc by dripping. 50% mortality was achieved by 8 days post-infection (d.p.i.) on injection of 10¹³ gc, 11 d.p.i. at 10⁹ gc, and 12 d.p.i. at 10⁵ gc. Administration by cannibalism led to a slower course of infection but full mortality was observed. No symptomatic individual recovered in any experiment. At 14 d.p.i., among larvae exposed to ZmBWV by feeding, viral load (by qPCR of NS1 gene) was 6.6×10¹² gc/mL in presymptomatic larvae and higher still at 2.2×10¹³ gc/mL and 1.7×10¹³ gc/mL in symptomatic and deceased larvae, respectively. Meanwhile, dead larvae infected by injection at 10¹⁵ or 10¹³ gc/mL had viral loads of 7.9×10¹² and 3.9×10¹² gc/mL, respectively. No sequence differences were identified in virus recovered from larvae experimentally inoculated with the strain UT-morio, confirming Koch's Fourth Postulate.

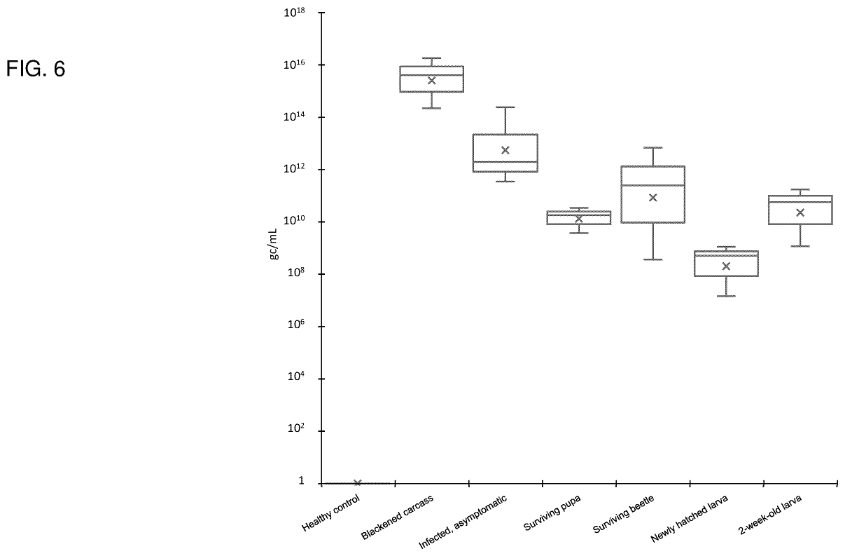

To assess the time course of natural infection, we obtained Z. morio individuals in various stages of life from an affected farm in New York, viz., 8-week-old symptomatic or asymptomatic larvae, surviving pupae and beetles, as well as one-week-old larvae that were the offspring of surviving beetles. Virus yield (by NS1 qPCR) varied extensively by life stage, with titers from ˜1×10⁹ gc/mL in newly hatched larvae to ˜2×10¹⁶ gc/mL for blackened carcasses (see FIG. 6). The virus yield of 8-week-old larvae also varied significantly, within about 1×10⁹ gc/mL to about 2.5×10¹⁶ gc/mL, depending on the absence, presence and severity of their symptoms. Surviving beetles exhibited a titer of about 7×10¹² gc/mL and the breeder reported that beetles who survived through pupation continued to reproduce at the farm.

Symptomatic larvae exhibited a dark area of miscoloration along the midgut prior to gross blackening. This, and the high viral load in larvae infected by feeding, suggested that the midgut plays a crucial role in the ZmBWV infection. We fixed and iodine-stained healthy and freshly-deceased larvae for microscale X-ray computed tomography (microCT). While healthy larvae had an intact midgut wall, the midgut wall of dead larvae was extremely thin and was disrupted by frequent fenestrations. The preserved ring-shaped structures suggest that the longitudinal muscles of the outer midgut wall remained intact, while the inner layer (which is composed mainly of columnar cells) was destroyed by ZmBWV infection. Not wishing to be bound by theory, it is possible that when infected per os, the virus somehow breaches the midgut wall. Lepidopteran densoviruses, for example, use transcytosis and then disseminate through the hemocoel and devastate fat bodies. However, ZmBWV likely directly infects the midgut epithelium as do many flaviviruses and alphaviruses as well as the distantly related bidnaviruses.

Next, the difference in virulence between the T. molitor and Z. morio-derived ZmBWV strains was investigated using the same inoculation experiments, using Z. morio as the host. Along with the reference strain UT-morio, we used purified virus of the T. molitor-derived “NJ2-molitor” strain. Fat body injections by the pathogenic strain UT-morio mirrored the results of the previous inoculation experiment i.e., all larvae died 10 to 21 d.p.i., in a dosage-dependent manner. Larvae inoculated by 10¹³ gc of the NJ2-molitor strain displayed ˜65% mortality by 3 d.p.i., albeit ˜30% of them survived beyond 30 d.p.i., the timepoint at which the experiment was terminated. Lower inoculation titers of 10⁹ gc and 10⁵ gc, resulted in 70% and 90% survival 30 d.p.i., respectively, suggesting that the large scale die-off of the highest inoculation titer may be due to acute viral toxicity.

The feeding experiment was repeated by offering the healthy larvae either blackened Z. morio larvae, infected by the strain UT-morio, or blackened T. molitor larvae, exhibiting titers of the strain NJ2-molitor of about 10¹⁶ gc/mL. Although the survival curve of the strain UT-morio infected group echoed the previous results of 100% mortality to set in at 25 d.p.i., we could not observe any symptomatic larvae in the NJ2-molitor-fed group, nor could we collect any carcasses, resulting in 100% survival at 30 d.p.i. These larvae were kept alive for another four months. There was no significant difference in the virus yield of the two earlier timepoints at 2.68×10⁹ gc/mL and 1.15×10¹⁰ gc/mL, respectively. The virus was still detectable at four months post inoculation, albeit at a low titer of 1.78×10⁷ gc/mL.

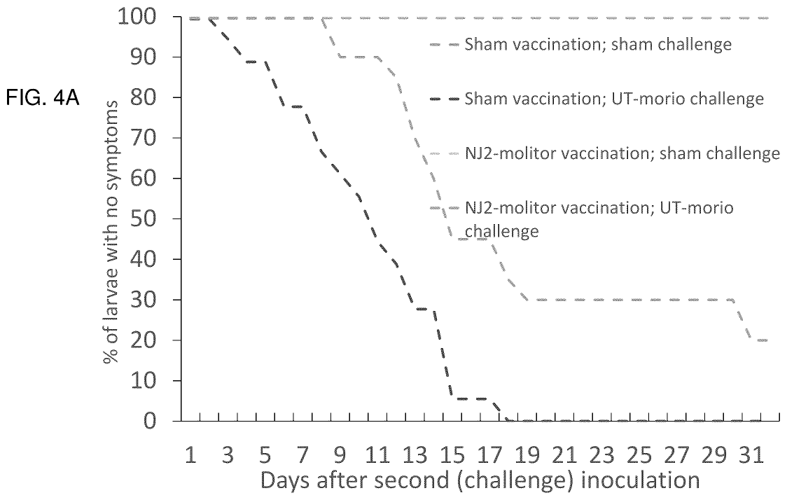

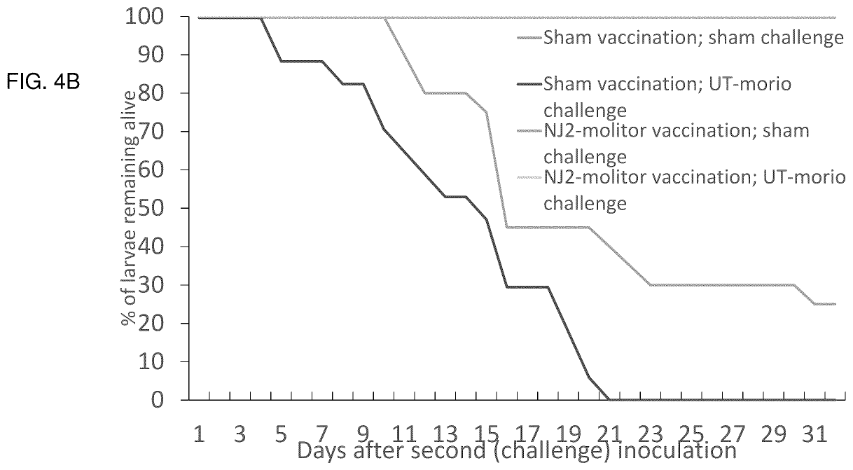

To investigate whether nonpathogenic strains of ZmBWV can be used to confer protection against disease and death, we inoculated the healthy larvae with the NJ2-molitor strain at a dose of 10⁹ gc by injection. The larvae were challenged at 21 d.p.i by injection with either the strain UT-morio at 10⁷ gc or with an equivalent volume of saline. Larvae that were inoculated exclusively by the strain UT-morio, reached 100% mortality 21 d.p.i. In case of the double-inoculated larvae, a 30% survival could be observed at the termination of the experiment (32 d.p.i.). This group also showed a seven-day-long delay in the onset of the first symptoms, compared to the single, strain UT-morio-inoculated treatment group. See FIG. 4.

### Example 3: ZmBWV Purification Method

Infected Z. morio larvae carcasses were subjected to sucrose cushion and sucrose step gradient purification. In the step gradient, which included fractions of 5-60% sucrose at 5% step intervals, two well-defined protein bands could be observed at the 20-25% and the 35-40% interfaces, respectively. See FIG. 1B, which shows virus purification from the Z. morio larvae from FIG. 1A, using a 5-60% sucrose step gradient. The sucrose step gradient resulted in two well bands due to particle accumulation of different buoyancy at the 20-25% (upper) and 35-40% (lower) interfaces by arrows). Both bands were collected, dialyzed, and plunge frozen on an electron microscopy grid. Isometric virus particles of approximately 26 nm in diameter were observed by cryoEM. This shape and diameter is consistent with DV infection. See FIG. 1C, which is a TEM micrograph showing empty ZmBWV particles from the band of the step gradient in FIG. 1B and FIG. 1D, which is a TEM micrograph showing genome packaging, i.e., full particles acquired from the lower fraction of the sucrose in step gradient shown in FIG. 1B. This putative DV was designated as ZmBWV. Genomic material was observed in particles from the lower buoyancy fraction but not the upper, more-buoyant fraction; the presence and buoyant separation of full and empty particles is not uncommon in non-enveloped viruses. Although ZmBWV predominated in both bands, the occasional flexivirus-shaped particle was observed. These were assumed to be bacteriophages infecting the Z. morio gut microbiome.

Upon subjecting the purified particles to SDS-PAGE, five bands corresponding to the approximate sizes of 85 kDa, 74 kDa, 65 kDa, 50 kDa and 48 kDa, could be observed. See FIG. 1E, an SDS-PAGE of heat-denatured protein, stain-infected own-defined (marked he upper red from ned with Coomassie brilliant blue, of the full fraction of purified ZmDV particles. The five putative capsid proteins are marked by arrows. Members of subfamily Densovirinae are known to express multiple structural proteins, with four VPs being the most common. From VP1 to VP4, respectively, these are incorporated into the GmDV capsid in a ratio of 1:9:9:41 and in the ratio of 1:11:18:30 into the AdDV capsid. Based on the VP transcripts of the type member of genus Blattambidensovirus, Blattella germanica densovirus (BgDV), the predicted masses of three potential VPs should correspond to 85.3, 69.7 and 56.3 kDa, yet they were shown to run significantly larger due to ubiquitination. Due to leaky scanning of VP transcript 2 and 3, BgDV expresses five VPs, with VP4 being the most abundant. The number and size of the ZmBWV VPs corroborates with this, further confirming that ZmBWV is a blattambidensovirus. However, ZmBWV appears to incorporate an approximately equal amount VP4 and VP5 major structural proteins into its capsid and possibly lacks the aforementioned posttranslational modifications characterized for BgDV.

### Example 4: Characterization of ZmBWV by Electron Microscopy

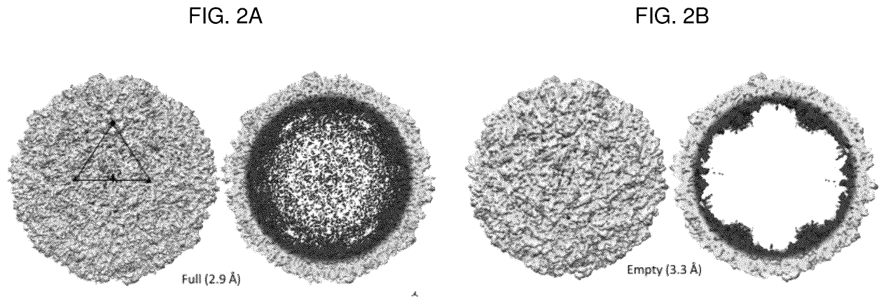

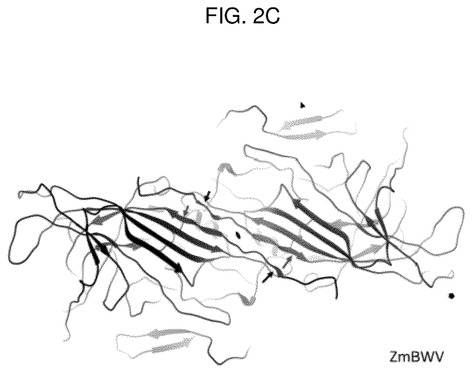

By means of electron microscopy, we identified a DV of genus Blattambidensovirus in connection with an outbreak of mass-mortality in captive Z. morio larvae, designated Zophobas morio black wasting virus (ZmBWV). The ZmBWV capsid has a densovirus-like structure with a unique surface morphology. The plunge-frozen grids with the viral particles were subjected to high-throughput cryoEM data collection to determine the atomic structure of the ZmBWV capsid by single-particle reconstruction. We resolved the ZmBWV capsid structure for both the genome packaging (full), particles and for the empty, high buoyancy particles, at the nominal resolutions of 2.9 A and 3.3 A, respectively. See FIG. 2A and FIG. 2B.

### Example 5. ZmBWV Genome and Structural Proteins

Because cryoEM had revealed that ZmBWV is a parvovirus, we knew the genome must be comprised of linear ssDNA, which is refractory to ligase-based next-generation sequencing (NGS) preparation but amenable to transposase-based NGS preparation. Parvoviral genomes are short and therefore need few total reads to achieve good coverage. We obtained a complete genome of the index case of ZmBWV by transposase-based NGS. The genome is 5,452 nt long, with I-shaped inverted terminal repeats (ITRs) of 180 nt at both termini. Its coding region harbored five major open reading frames (ORFs) over the (+) and (−)-sense frames, suggesting an ambisense replication strategy. Three of these, located on the right strand, were homologues of the NS1, NS2 and NS3 proteins of DVs classified to genus Blattambidensovirus of the Densovirinae subfamily (protein sequence identity of 45-98%, according to homology searches by BLASTP).

Blattambidensoviruses, such as the type species, Blattella germanica densovirus (BgDV), express three capsid proteins encoded by two ORFs: cap1 and cap2. While cap2 gives rise to major capsid protein VP3, cap1 provides the N-terminal extensions to these in order to express minor VP2 and VP1 via alternative splicing. ZmBWV, contains homologues of cap1 and cap2 (amino acid identity of 41% and 49.5%, respectively, with their BgDV counterparts). Analyzing either empty capsids or full virions by SDS-PAGE, four protein bands were observed at sizes of 85 kDa, 74 kDa, 50 kDa and 48 kDa, with the 50 kDa fraction being the most abundant. Protein sequencing by tandem mass spectrometry (MS/MS) revealed that the three smaller-sized fractions (designated as VP4, VP3 and VP2) all were products of cap2 exclusively, while only the largest fraction encompassed the almost complete cap1. Consequently, we designated this protein VP1. See sequences above.

### Example 6. Complete Genome Sequencing and Phylogenetic Calculations for ZmBWV

Viral DNA was extracted either from purified virus particles or directly from ZmBWV-infected insects. In the first case 0.25 mg of full ZmBWV particles were incubated for three hours in 1× TE puffer, pH 8.7 (10 mM tris-HCl, 1 mM EDTA) supplemented with 10 μl 10% sarcosyl and 4 μl proteinase K in 10 mg/ml concentration. The DNA was extracted by the DNeasy™ kit from Qiagen™. To isolate viral DNA directly from insects, we pooled five individuals in a 5-ml conical tube and homogenized them in 1 mL 1× TE buffer, using a handheld homogenizer with a sterile pestle. DNA was isolated from 100 μL homogenized suspension, which was digested overnight with the same components as the virions. The isolation step was carried out by the Monarch PCR & DNA Cleanup kit from New England Biolabs™, utilizing the single-stranded DNA specific protocol. The acquired DNA preps were shipped to a commercial Oxford nanodrop sequencing service, provided by Plasmidsaurus™ (Eugene, OR, USA) where they were processed as linear amplicons.

The obtained complete genomes were aligned by T-Coffee (Notredame) and converted to nexus format using Unipro Ugene. Model selection was carried out by the nucleic acid model selection subroutine of IQ-Tree (Nguyen™). Phylogenetic inference was calculated by BEAST.

The sequence of Zophobas morio black wasting virus, strain “UT-morio,” follows:
(SEQ ID NO: 1)
gacgaccagatctatttatacccaatggtgaccttggaaacctggttaccatgtaaattttctatttactggtata
ccatggtttccaaggtcaccattgggtataaatagatctggtcgtcgtcgcagtcgtcagtgataagccagg
ggcttatgggcaagcaccaagtaagggggataaagtgaagtgtataaatagtgttcaacatgtaagtagctc
attctgttgttgtatttcgtgcagtgcagtaatactttggtttggtaagtaggtttaaacataagaagatatatcac
cctcagagcttgtatgacatgactaagaagaaggtaagagaggtgtacaagaataattggaacttgctggta
ctaccgaatcgtatccagagggaactactactagattggttaagatgtgacgagacaattccggagagtgat
gacgatgtggaacgaatagtggcacgtatggaaaggggatgggaggcattgaagccctttggttcgacta
catttgtgtacttgatgagattaccagatgaagtaccgccatttgcacatgaaaggaatcatatcatttgggact
tttacttgtggtatgaacaagggcgagaaaagaaaatttgtgaaccgtgttattccgggaagagtcgtttctac
tgtccaggatctgctaatgaatggttagaaaaaggatgggtatttaaacgcgtagagaatcactccatgattg
atggggacagattgttacaggagttgatatgggatgaagacaactggtgcagtttatgtcttgttgagccatta
tggatccatatattggatgatgatgattgtttgtttgattatgactatcatttgaagagaaggcgtacatggagtg
actcaagctctgaggacagtgatattgattattgtaaacatactgttatgcaagggattcgtatgaaccccactt
tgtataaatttattaatgaataaagcaagtgttatagaattgttgtttcattacagatgaataatgacgaccactttt
gggaatattttgacagtactctgggagatgcttccggacgagtgggcgaatcatccgggagtatggtggaa
gattatggatacgataccactggaccaacaatgcaagaatgcgatgttacagctaatagggcgttggagca
agaattacaaacaatggtcgaccggttcgttacccgccttgaaaaagaagattggcaagacagcggatact
atgtgtcagatgtctttgcctgcgaatcaattggacgagctcagggattggctaagcgaatggctgaacgag
cgggaaatttccgacgaggacttatccttatatctatccacagcgatcaggatggcagtgcccatgtccaca
ccatccacagctgcgcctacgcaaacagggcgtgcaggtgctacttcaaagcgttccccgaagcgcaaga
ggacgctagacgacttcttcgaaagcctccacccatcgaaacgttcaagcgaagcgattgggaaaatatca
caaagtatttttgtacgaaagggagacgagcaacgttcgctaaaatcaacggtgccatacaaagacttcctc
ttgaaattacaaatctatccgacattatcctatcaggccaagttgaaggaggatcacactcaggcctggaga
actgccacgacccgcttgacaataacaatgaacgaaaacgaggaattgaaccgaaaggtgatggaggtac
tcgaagtcgcaaaagaagaggtgttggcaacgcaggaggagatggaggaatcagaggggacacagga
gtaattcttgatttaattaagaggtatgctgtttgtccattatctgagatagtatatactcgcgagtacttggaaaa
tcgtattgcagttaaaaggttagatgatagggatgtaaagaacgctatagattgccatgctgctattattaatac
ttggaatagagaggattatgttaagttctatgaggacccaaacactgttaaaatttggagtgcacgtagtattg
acttggtggaattatattatttaactgataatgaatcgagggatattattaataaactgctggattaccaatgtgg
gttagctaagaaacagtttgtaacagatttgattaatgtaacagacatgaatattcccaagtgtaattgctttcta
attgttagccctccaagtgctggcaagaactttctatgcgatgctattaaagattactatcttaatgttggacaaa
tgcagaaccctaataaatataataccttcgcgtatcaggattgtcacaatagacgacttcttatttggaatgaac
caaactacgaacctagagaaacggagaatttaaagatgttatttggaggggataacttgtctgctaatgttaa
gtgtaaacctcaagctaatgtaaagcgtactccagtaatatgtatgagtaatgttgtacccagatttgctaatca
tgaagcatttgctgacagagttattacatattattggaatgctgctccgttcttgaaagaggtaataaaaaaacc
acgacctgattctgtaatgaatttattatatgaaatatacaattcataattatggttttacagttttaatcatttgcatc
ctgccatatacattaggattatcatatactttagcaataggacgaccatcatttgcaaaatattgaagttgagatt
tggtattggccgagtaacatccgcccttaatgtatgtgtaagggtcgacactctctgttgttagtacacaatca
acttcgaagtagccttgtgcgtctagccaggaattagcttgagtagtttcatcaatggtagttaacttgggaac
agctcttatgccaacatttatagatggcatttgagtgtcatgtactgtctcaacatttacctcctcgaatatacctc
cttgttcaatagggactcgaagatagttctgttcctcatcaaagaatgttgtatctgcattgggaccctgtatata
tttgcgaggtgcattacgtacttgtgttggtggtgtagcttgtgaggaatcggttgttttcgctgccgtcacttca
ttctttgttcccgcaggataactagcattatagttttgagtgatcaagttgtttggtactggtgcaaactgtggta
ccagaggtgcgtagctaaaatcgtaatccatggcaactacatctgtgttgatacatgcacttgcatcgaattct
tctataaagtttttgtatggtggaaaccccgccaatatcttagtaccagtagtagcccttgcatcattagtgtaaa
tggttaaatagtcctgtagatatatctctgcgccagttgcatctgctggtggtttcttagcaaagtctggactgtc
attatcgtaaccatacatagcaatcttgagaccatctcgatattcgtaggatgtcaatgttgcgaatcccgtag
gttgcatgggttcagtatcagaatatgtatactttctgtttgtagagcatataaaaggaatacttctaataccctta
gcaacttgcagaaatttgttttgattcagtgtagcattagccgtttgagtatctccagtttgaaatgcaactctag
tattccatgttctaatacgtactgaagccgacttagcgaacgtcccaggatagttcttcattcgattatattctgc
tggactcatataaaagaataagtattcccatggaattgatgccattcctgtggttaatgcccatcttgctggtctt
tctccgctagctgcctcggccaagatgacgttggcatttgctgaagttaggaatctccatttctttctataagttt
gttgaaacttttcaacatgaagtccaattggacgtaatatagctgtagctccgccttccccactacctcctctag
ccataccatccaaattgcctcctgttcctggtaaacttgtcccagacttggataccttactcggcggtccgcta
ccatcactacctcgcttggatccactactagagctagatacttgagccatttctacgtccgccatgtttggttgt
ggtgcgttacttggtccggcgtctcttaaatcttgtctatctcttacttgttgtaaaaaatcgtctactccgtcttct
gattgctgactgggttgttccgcctcctggggtgaccctaaaagatccaaatcaaagttatcaattgcatcag
cctgagttaaaggtaggtcgtcatccccaggaggatcaaaatattctccacgtcttactctagctaggttccat
tgttccatagcatagcgtctttgtccatcattcatactatcccaattaggacgatcttggggattcggacccagc
ggtctaactctaggcattgcttaccgtaatatacttttccacttagacgttcgactgcgtgcttaactcccaacc
cgactcctcccactgccgcgtgtatcctagatataggatcctgaccctggattgcctcgtgtgcgaattgtga
gattgcttctctatccgctgacagcacgtcgctatcgctcttagcttgttgataatgtaagtcgtgaccctgcgc
aataaggtcagctctgttggtcgcgggacgtactggatttccaggtccgatattgttcgaaaacggtaaaact
agtccgttccgtggtggttctccccttgctagattggcagaatctcttctcgcctgcgcttccctttgttcttgctt
cttcgccttgtttagtcccctgcgaggatttactagtctccagtgttctggatatttagccttagtgtccttattcca
aggatctccgtacggtttgatcttctcctgtcgctctttcaaccccaaacgtctcaattcccaaggaacggctg
cgtagtaatcgtctcgattaccaaatattgctctcgataaagggttgttagggtcagctgctgctcttcccaatt
gcccttcgaatattggttgatattggctacctgctccaataaccgcacccccaacacgtggtgtattaggtacc
ttcaatttactgtcacttccggcactggagatactactattagacgagttaataggaatagatacgtatccaccc
ctacgtcgcacccctgtacttgtattgggttgattaaacacactctcttcactactgctgggggtaatagtgctc
agctcgatgtactcggacatactgactggaaataacctgcgcatacatatttatatccatgttgatcccccttac
ttggtgcttgcccataagcccctggcttatcactgacgactgcgacgacgaccagatctatttatacccaatg
gtgaccttggaaaccggttaccatgtaaattttctatttacggtataccatggtttccaaggtcaccattgggtat
aaatagatctggtcgtcgtc
The sequence of Zophobas morio black wasting virus, strain “FL-morio” follows:
(SEQ ID NO: 2)
aaccggttaccatgtaaattttctatttacggtataccatggtttcaaggtcaccatttgggtataaatagatct
ggtcgtcgtcgcagtcgtcagtgataagccaggggcttatgggcaagcaccaagtaagggagataaagt
gaagtgtataaatagtgttgaacatgtaagtagctcattctgttgttgtattttatacagtgcagtaatactttgc
tttggtaagtaggtttagacataagaagatatatcaccctcagagcttgtatgacattactaagaagaaggta
agagaggtgtacaagaataattggaacgagacgaaggtactagcgaagagtatccagagggaactact
actagattggttaagatgtgacgagacaattcctgagagtgatgacgatgtggaacgaatagtggcacgt
atggaaaggggatgggaggcattgaagccctttggtccgactacatttgtgtacttgatgagattaccaga
tgaagtaccgccatttgcacatgaaaggaatcatatcatttgggacttctatttgtggtatgaacaagggcg
agaaaagaaaatttgtgaaccgtgttattccgggaagagtcgtttctaccgtccaggatctgctaatgaatg
gttagaaaaaggatgggtatttaaacgcgtagagaatcactccatgattgatggggatagattgttagagg
atttgatatgggatgaagacaactggtgcagtttatgtattgttgagccattatggatccatatatttgatgatg
atgattgtttgtttgattatgactatcatttgaagagaaggcgtacatggagtgactcaagctctgaggacag
tgatattgattattgtaaacatactgttatgcaagggattcgtataaaccccactttgtataaatttattaatgaat
aaagcaagtgttatagaattgttgtttcattgcagatgaataatgacgaccacttttgggaatattttgacagt
actctgggagatgcttccggacgagtgggcgaatcatccgggagtatggtggaagattatggatacgata
ccactggaccaacaatgcaagagtgcgatgttacagctaatagggcgttggagcaagaattacaaacaa
tggtcgaccggttcgttacccgccttgaaaaagaagattggcaagacagcggatactatgtgtcagatgtc
tttgcctgcgaatcaattggacgagctcagggattggctaagcgaatggctgaacgagcgggaaatttcc
gacgaggacttatccttatatctatccacagcgatcaagatggcagtgcccatgtccacaccatccacagc
tgcgcctacgcaaacagggcgtgcaggtgctatttcaaagcgttccccgaagcgcaagaggacgctag
acgacttcttcgaaagcctccatccatcgaaacgttcaagcgaagcgattgggaaaatatcacaaagtatt
tttgtacgaaagggagacgagcaacgttcgctaaaatcaacggtgccatacaaagacttcctcttgaaatt
acaaatctatccgacattatcctatcaggccaagttaaaggaggatcacactcaggcctggagaactgcc
acgacccgcttgaccatcacgatgaacgaaaacgaggaattgaaccgaaaggtgatggaagtactcga
agccgcaaaagaagaggtgttggcaacgcaggaggagatggaggaatcagaggggacacaggagta
attcttgacttaattaagaggtatgctgtttgtccattatctgagatagtatatactcgcgagtacttggaaaat
cgcattgcagttaaaaggttagatgatagggatgtgaagaacgctatagattgccatgctgctattattaata
cttggaatagagaggattatgttaagttctatgaggacccaaacactgttaaaatttggagtgcacgtagtat
tgacttggtggagttatattatttaactgataatgaatctagagacattattaataaactgcttgattaccaatgt
gggttagctaagaaacagtttgtaacggatttgattaatgtaacagacatgaatattcccaagtgtaactgct
ttctaattgttagccctccaagtgctggcaagaactttctatgcgatgctattaaagattactatcttaatgttg
gacaaatgcagaaccctaataaatataataccttcgcgtatcaggattgtcacaatagacgacttcttatttg
gaatgaaccaaactacgaacctagggaaacggagaatttgaagatgttatttggaggggataacttgtctg
ctaatgttaagtgtaaacctcaagctaatgtaaagcgtactccagtaatatgtatgagtaatgttgtacccag
atttgctaatcatgaagcatttgctgacagagttattacatattattggaatgctgctccgtttttgaaagaggt
aataaaaaaccacgacctgattctgtaatgaatttattatatgaaatatacaactcataattatggttttacagtt
ttaatcatttgcatcctgccatatacattaggattatcatatactttagcaataggacgaccatcacttgcaaaa
tattgaagttgagatttggtattggccgagtaacatccgcccttaatgtatgtgtaagggtcgacactctctgt
tcttagtacacaatcaacttcgaagtagccttgtgcgtctagccaggaattagcttgagtagtttcatcaatg
gtagttaacttgggaacagctcttatgccaacatttatagatggcatttgagtgtcatgtactgtctcaacattt
acctcctcgaatatacctccttgttcaatagggactcgaagatagttctgttcctcatcaaagaatgttgtatct
gcatggggaccctgtatatatttgcgaggtgcattacgcacttgtgttggtactgtagcttgtgaggaatcg
gttgttttcgctgccgtcacttcattctttgttcccgcaggataactaccattatagttttgagtaatcaagttgtt
tggtactggtgcaaactgtggtaccagaggtgcgtagctaaaatcgtaatccatagcaactacatctgtgtt
tatacatgcacttgcatcgaattcttctataaagtttttgtatggtggaaaccccgccaatatcttagtaccagt
agtagcccttgcgtcattagtgtaaatggttaaatagtcctgtagatatatctctgcgccagttgcatctgctg
gtggtttcttagcaaagtctggactgtcattatcgtaaccgtacatagcaatcttgagaccatctcgatattgg
taggatgtcaatgttgcgaatccagtaggttgcataggttcagtatcagaatatgtatactttctgtttgtaga
gcatataaaaggaatacttctaatccccttagccacttgtaagaacttattctgatttagtgtagcattagctgt
ttgagtatctccagtttggaatgcaactcttgtgttccacgtcctaatacgtactgaggcggacttagcaaac
gtaccaggatagttcttcattctgttatattctgctggactcatataaaagaataagtattcccatggaattgat
gccattcctgtcgttagtgcccatcttgctggtctgtctccgctagccgcttcagccaatatgacgtttgcatt
tgctgaagtcagaaacctccatttctttctataagtctgttgaaacttctcaacatgaagtccaattggactaat
atagctgtagctccgccttccccactacctcctctaaccatgccatctaagttgcctccagttccagggaga
ctcgtccctgacttggataccttactcggcggtccgctaccatcactgcctcgcttcgatccaccgctggaa
cttgatacttgagacatttctacgtccgccatgtttggttgtggtgcgttacttggtccggcgtctcttaaatctt
gtctatctcttacttgttgtaaaaaatcgtctactccgtcttctgattgctgactgggttgttccgcctcctggg
gtgaccctaaaagatccaaatcaaaattatctattgcatcagcctgagttaatggttggtcgtcatccccag
gaggattaaagtattccccacgtcttactctagctaggttccattgctccatagcataacgtctttgtccctcg
ttcatactatcccaattaggacgatcctgaggattcggacccaacggtctaactctaggcattgcttaccgt
agtatacttttccgcttaaatgttcgactgcgtgcttaactcccagcccgactcctcccactgccgcgtgtat
cctagatataggatcctgaccctggattgcctcgtgtgcgaattgtgagattgcttctctatccgctgacagc
acgtcgctatcgctcttagcttgttgataatgtaagtcgtgaccctgcgcaataaggtcagctctgttgctcg
cgggacgtatgggatttccaggtccgatattgttcgaaaatggtaaaactagtccgttccgtggtggttctc
cccttgctagattggcagaatctcttctcgcctgtgcttccctttgttcttgcttcttcgccttgtttagtcccctg
cgaggatttactagcctccagtgttctggatatttggctttagtgtccttattccaagggtctccgtagggtttg
atcttctcttgtcgatctttcaaccccaatcgtctcaattcccaaggaacggctgcgtagtaatcgtctcgatt
gccaaaaattgctcttgacaaggggttattaggg
The sequence of Zophobas morio black wasting virus, strain “LA-molitor” follows:
(SEQ ID NO: 3)
gacgacgaccagatctatttatacccaatggtgaccttggaaaccatggtataccatgtaaatagtaaattta
catggtataccatggtttccaaggtcaccattgggtataaatagatctggtcgtcgtcgcagtcgtcagtgat
aagccaggggcttatgggcaagcaccaagtaagggagataaagtgaagtgtataaatagtgttcaacat
gtaagtagctcattctgttgttgtatttcgtgcagtccagtaacactttggtttggtaagtaggtttaaacataa
gaagatatatcaccctcagagcttgtatgacattactaagaagaaggtaagagaggtgtacaagaataatt
ggaacgagacgaaggtactaccgaggactatccagagggaactattactagattggttaagatgtgacg
agaaaatttctgagagtgatgacgatgtggaacgaatagtggcacgtatggaaaggggatgggaggcat
tgaagccctttggtccgactacatttgtgtacttgatgagattaccagatgaagtaccgccatttgcacatga
aaggaatcatatcatttgggacttctatttgtggtatgaacaagggcgagaaaagaaaatttgtgaaccgtg
ttattccgggaagagtcgtttctaccgtccaggatctgctaatgaatggttagaaaaaggatgggtatttaaa
cgcgtagagaatcactccatgattgatggggacagattgttagaggatttgatatgggatgaagacaactg
gtgcagtttatgcattgttgagccattatggatccatatatttgatgatgatgattgtttgtttgattatgactatc
atttgaagagaaggcgtacatggagtgactcaagctctgaggacagtgatattgattattgtaaacatactg
ttatgcaagggattcgtataaaccccactttgtataaatttattaatgaataaagcaagtgttatagaattgttgt
ttcattacagatgaataatgacgaccacttttgggaatattttgacagtactctgggagatgcttccggacga
gtgggcgaatcatccgggagtatggtggaagattatggatacgataccactggaccaacaatgcaagaa
tgcgatgttacagctaatagggcgttggagcaagaattacaaacaatggtcgaccggttcgttacccgcct
tgaaaaagaagattggcaagacagcggatactatgtgtcagatgtctttgcctgcgaatcaattggacgag
ctcagggattggctaagcgaatggctgaacgagcgggaaatttccgaagaggacttatcctcatatctatt
cacagcgatcaagatggcggtgcccatatccacaccatccacagctgcgcctacgcaaacagggcgtg
caggtgctacttcaaagcgttccccgaagcgcaagaggacgctagacgacttcttcgaaagcctccacc
catcgaaacgttcaagcgaagcgattgggaaaatatcacaaagtatttttgtacgaaagggagacgagca
acgttcgctaaaatcaacggtgccatacaaagacttcctcttgaaattacaaatctatccgacattatcctatc
aggccaagttgaaggaggatcacactcaggcctggagaactgccacgacccgcttgacaataacgatg
aacgaaaacgaggaattgaaccgaaaggtgatggaggtactcgaagtcgcaaaagaagaggtgttggc
aacgcaggaggagatggaggaatcagaggggacacaggagtaattcttgatttaattaagaggtatgct
gtttgtccattatctgagatagtatatactcgcgagtacttggaaaatccccttgcagttaaaaggttagatga
tagggatgtaaagaacgctatagattgccatgctgctattattaatacttggaatagagaggattatgttaag
ttttatgaggacccaaaaactgttaaaatttggagtgcacgtaatattgactcggtggagttatattatttaact
gataatgaatctagagatattattaataaactgcttgattatcaatgtgggttagctaagaaacagtttgtaac
ggacttgattaatgtaacagacatgaatattcccaagtgtaattgctttctaattgttagccctccaagtgctg
gcaagaactttctatgcgatgctattaaagattactatattaatgttggacaaatgcagaaccctaataaatat
aataccttcgcgtatcaggattgtcacaatagacgacttcttatttggaatgaaccaaactacgaacctagg
gaaacggagaatttgaagatgttatttggaggggataacttgtctgctaatgttaagtgtaaacctcaagcta
atgtaaagcgtactccagtaatatgtatgagtaatgttgtacccagatttgctaatcatgaagcatttgctgac
agagttattacatattattggaatgctgctcctttcttaaaagaggtaataaaaaaaccacgacctgattctgt
aatgaatttattatatgaaatatacaattcataattatggtttcacagttttaatcatttgcatcctgccgtatacat
taggattatcatatactttagcaataggacgaccatcacttgcaaaatattgaagttgagatttggtattggcc
gagtaacatccgcccttaatgtatgtgtaagggtcgacactctctgttgttaatacacaatcaacttcgaaat
agccttgtgcgtctagccaggaattagcttgagtagtttcatcaatggtagttaacttgggaacagctcttat
gccaacatttatagatggcatttgagtgtcatgtactgtctcaacatttacctcctcgaatataccaccttgttc
aatagggactcgaagatagttctgttcctcatcaaagaacgttgtatctgcatggggaccctgtatatatttg
cgaggtgcattacgtacttgtgttggtgctgtagcttgtgaggaatcggttgttttcgctgccgtcacttcatt
ctttgttcccgcaggataactaccattatagttttgagtaatcaagttgtttggtactggtgcaaactgcggta
ccagaggtgcatagctaaaatcgtaatccatagcaactacatctgtgtttatacatgcacttgcatcgaattc
ttctataaagtttttgtatggtggaaaccccgccaatatcttagtaccagtagtagcccttgcatcattagtgta
aatggttaaatagtcctgcagatatatctctgcgccagttgcatctgctggtggtttcttagcaaagtctgcac
tgtcattatcgtaaccatacatagcaatcttgagaccatctcgatattggtaggatgtcaatgttgcgaatcc
cgtaggttgcatgggttcagtatcagaatatgtatactttctgtttgtagaacatataaaaggaatacttctaat
acccttagcaacttgcagaaatttgttttgattcagtgtagcattagccgtttgagtatctccagtttggaatgc
aactcttgtgttccacgtcctaatacgtactgaagcggacttagcaaacgtaccaggatagtttttcattctgt
tatattctgctggactcatatagaagaataagtattcccatggaattgatgccattcctgtcgttagcgcccat
cttgctggtctttctccgctagctgcctcggccaagatgacgttggcatttgctgaagttaggaatctccattt
ctttcggtatgtttgttgaaatttttcgacgtgtaatccaattggacgtaatatagctgtagctccgccttcccc
actacctcctctaaccataccatccaaattgcctcctgttcctggtaaacttgtcccagacttggataccttac
tcggcggtccgctaccatcactacctcgcttggatccactactagagctagatactcgagccatttctacgt
ccgccatgtttggttgtggtgcgttacttggtccggcgtctcttaaatcttgtctatctcttacttgttgtaaaaa
atcgtctactccgtcttccgattgctgactgggttgttccgcctcctggggtgaccctaaaagatccaaatca
aagttatcaatcgcatcagcctgagttaaaggtaggtcgtcatccccaggaggatcaaagtattccccacg
tcttactctagctaggttccattgttccatagcataacgtctttgtccctcattcatactatcccaattaggacga
tcctgaggattcggacccaacggtctaactctaggcattgcttaccgtagtatacttttccgcttaaatgttcg
actgcgtgcttaattcccagcccgattcctcccactgccgcgtgtatcctagatataggatcctgaccctgg
attgcctcgtgtgcgaattgtgagattgcttctctatccgctgacagcacgtcgctatcgctcttagcttgttg
ataatgtaagtcgtgaccctgcgcaataaggtcagctctgttggtcgcgggacgtattggatttccaggtcc
gatattgttcgaaaacggtaaaactagtccgttccgtggtggttctccccttgctagattggcagaatctcttc
tcgcctgtgcttccctttgttcttgcttcttcgccttctttagtcccctgcgaggatttactagtctccagtgttct
ggatatttagccttagtgtccttattccaaggatctccgtacggtttgatcttctcctgtcgctctttcaacccca
aacgtctcaattcccaaggaacggctgcgtagtaatcgtctcgattaccaaatattgctctcgataaagggt
tgttagggtcagctgctgctcttcccaattgcccttcgaatattggttgatattggctacctgctccaataacc
gcacccccaacacgtggtgtattaggtactctcaatttactgtcactgccggcactggagatactactatta
gacgagttaataggaatagatacgtatccacccctacgtcgcacccctgtacttgtactgggttgattaaac
acactctcttcactactgctgggggtaatagtgctcagctcgatgtactcggacatactgactggaaataac
ctgcgcatacatatttatatccatgttgatcccccttacttggtgcttgcccataagcccctggcttatcactga
cgactgcgacgacgaccagatctatttatacccaatggtgaccttggaaaccatggtataccatgtaaatat
aaaatttacatggtataccatggtttccaaggtcaccattgggtataaatagatctggtcgtcgtc
The sequence of Zophobas morio black wasting virus, strain “NJ1-molitor” follows:
(SEQ ID NO: 4)
agatctatttatacccaatggtgaccttggaaaccatggtataccatgtaaatagtaaatttacatggtatacc
atggtttccaaggtcaccattgggtataaatagatctgctcgtcgtcgcagtcgtcagtgataagccaggg
gcttatgggcaagcaccaagtaagggagataaagtgaagtgtataaatagtgtttaacatgtaagtacctc
attctgttgttgtatttcgtgcagtgcagtaatactttggtttggtaagtaggtttaaacataagaagatatatca
ccctcagagcttgtatgacattactaagaagaaggtaagagaggtgtacaagaataattggaacgagacg
aaggtgctaccgaagactatccagagggaactactactagattggttaagatgtgacgagaagatttctga
gagtgatgacgatgtggaacgaatagtggcacgtatggaaaggggatgggaggcattgaagccctttg
gtccgaccgcatttgtgtacttgatgagattaccagatgaagtaccgccatttgcacatgaaaggaatcata
tcatttgggacttttatttgtggtatgaacaagggcgagaaaagaaaatttgtgaaccgtgttattccgggaa
gagtcgtttctaccgtccaggatctgctaatgaatggttggaaaaaggatgggtatttaaacgcgtagaga
atcactccatgattgatggggacagattgttagaggatttgatatgggatgaagacaactggtgcagtttgt
gtattgttgagccattatggatccatatacttgatgatgatgattgtttgtttgattatgactatcatttgaagaga
agacgtacatggagtgactcaagctctgaggacagtgatattgattattgtaaacatactgttatgcaaggg
attcgtataaaccccactttgtataaatttattaatgaataaagcaagtgttatagaattgttgtttcattacagat
gaataatgacgaccacttttgggaatattttgacagtactctgggagatgcttccggacgagtgggcgaat
catccgggagtatggtggaagattatggatacgataccactggaccaacaatgcaagaatgcgatgttac
agctaatagggcgttggagcaagaattacaaacaatggtcgaccggttcgttacccgccttgaaaaagaa
gattggcaagacagcggatactatgtgtcagatgtctttgcctgcgaatcaattggacgagctcagggatt
ggctaagcgaatggctgaacgagcgggaaatttccgacgaggacttatccttatatctatccacagcgat
caggatagcagtgcccatgtccacaccatccacagctgcgcctacgcaaacaggtcgtgcaggtgctac
ttcaaagcgttccccgaagcgcaagaggacgctagacgacttcttcgaaagcctccacccatcgaaacg
ttcaagcgaagcgattgggaaaatatcacaaagtatttttgtacgaaagggagacgagcaacgttcgcta
aaatcaacggtgccatacaaagacttcctcttgaaattacaaatctatccgacattatcctatcaggccaagt
tgaaggaggatcacactcaggcctggagaactgccacgacccgcttgacaataacgatgaacgaaaac
gaggaattgaatcgaaaggtgatggaggtactcgaagtcgcaaaagaagaggtgttggcaacgcagga
ggagatggaggaatcagaggggacacaggagtaattcttgatttaattaagaggtatgctgtttgtccatta
tctgagatagtatatactcgcgagtacttggaaaatcccattgcagttaaaagattagatgatagggatgta
aagaacgctatagattgccatgctgctattattaatacttggaatagacaggattatgttaagttttatgagga
cccaaacactgttaaaatttggagtgcacgtaatgttgactcggtagagttatattatttaactgataatgaat
ctagagatattattaataaactgcttgattatcaatgtgggttagctaagaaacagtttgtaacggatttgatta
atgtaacagacatgaatattcccaagtgtaattgctttctaattgttagccctccaagtgctggcaagaacttt
ctatgcgatgctattaaagattactatattaatgttggacaaatgcagaaccctaataaatataatactttcgc
gtatcaggattgtcacaatagacgacttcttatttggaatgaaccaaactacgaacctagggaaacggaga
atttgaagatgttatttggaggggataacttgtctgctaatgttaagtgtaaacctcaagctaatgtaaagcgt
actccagtaatatgtatgagtaatgttgtacccagatttgctaatcatgaagcatttgctgacagagttattac
atattattggaatgctgctcctttcttaaaagaggtaataaaaaagccacgtcctgattctgtaatgaatttatt
atatgaaatatacaattcataattatggttttacagttttaatcatttgcatcctgccatatacattaggattatcat
atactttagcaataggacgaccatcacttgcaaaatattgaagttgagatttggtattggccgagtaacatcc
gcccttaatgtatgtgtaagggtcgacactctctgttgttagtacacaatcaacttcgaagtagccttgtgcgt
ctagccaggaattagcttgagtagtttcatcaatggtagttaacttgggaacagctcttatgccaacatttata
gatggcatttgagtatcatgtactgtctcaacatttacctcctcgaatataccaccttgttcaatagggactcg
aagatagttctgttcctcatcgaagaatgttgtatctgcgtggggaccctgtatatatttgcgaggtgcattac
gtacttgtgttggtgctgtagcttgtgaggaatcggttgttttcgctgccgtcacttcattctttgttcccgcag
gataactaccattataattttgagtaatcaagttgtttggtactggtgcaaactgcggtaccagaggtgcgta
gctaaaatcgtaatccatagcaactacatctgtgttgatacatgcacttgcatcgaattcttctataaagttttt
gtatggtggaaaccccgccaatatcttagtaccagtagtagcccttgcatcattagtgtaaatggttaaatag
tcctgtagatatatctctgcgccagttgcatctgctggtggtttcttagcaaagtctttattatcgttatcgtaac
catacatagcagtcttgagaccatctcgatattggtaggatgtcaatgttgcgaatcccgtaggttgcatgg
gttcagtatcagaatatgtatactttctgtttgtagaacatataaaaggaatacttctaatacccttagcaactt
gcagaaatttgttttgattcagtgtagcattagccgtttgagtatctccagtttgaaatgcaactctagtattcc
atgttctaatacgtactgaagccgacttagcaaacgtaccaggatagttcttcattcgattatattctgctgga
ctcatataaaagaataagtattcccatggaattgatgccattcctgtcgttagcgcccatcttgctggtctttct
ccgctggccgcttcagccaatatgacgtttgcatttgctgaagtcagaaacctccatttctttctataagtttgt
tgaaacttctcaacatgaagtccaattggacgtaatatagctgtagctccgccttccccactacctcctctaa
ccatgccatccaaattgcctccagttccagggagactcgtccctgacttggataccttactcggcggtccg
ccaccaccactgcctcgcttcgatccaccgctagaacttgatacttgagacatttctatgtctgccatgtttg
gttgtggtgcgttacttggtccggcgtctcttaaatcttgtctatctcttacttgttgtaaaaaatcgtctactcc
gtcttctgattgctgactgggttgttccgcctcctggggtgaccctaaaagatccaaatcaaagttatcaatc
gcatcagcctgagttaaaggtaggtcgtcatccccaggaggatcaaagtattccccacgtcttactctagc
taggttccattgttccatagcataacgtctttgtccctcattcatactatcccaattaggacgatcctgaggatt
cggacccaacggtctaactctaggcattgcttaccgtagtatacttttccacttaaatgttcgactgcgtgctt
aactcccagcccgattcctcccactgccgcgtgtatcctagatataggatcctgaccctggattgcctcgtg
tgcgaattgtgagattgcttctctatccgctgacagcacgtcgctatcgctcttagcttgttgataatgtaagt
cgtgaccctgcgcaataaggtcagctctgttggtcgcgggacgtattggatttccaggtccgatattgttcg
aatacggtaaaactagtccgttccgtggtggttctccccttgctagattggcagaatctcttctcgcctgtgct
tccctttgttcttgctttttcaccttctttagtcccctgcgaggatttactagtctccagtgttctggatatttagcc
ttagtgtccttattccaaggatctccgtacggtttgatcttctcctgtcgctctttcaaccccaaacgtctcaatt
cccaaggaacggctgcgtagtaatcgtcccgattaccaaatatcgctctcgataaagggttcctagggtca
gctgctgctcttcccaattgcccttcgaatattggttgatattggctacctgctccaataaccgcacccccaa
cacgtggtgtattaggtactctcaatttactgtcacttccggcactggagatactactattagacgagttaata
ggaatagatacgtatccacccctacgtcgcacccctgtacttgtactgggttgattaaacacactctcttcac
tactgctgggggtaatagtgctcagctcgatgtactcggacatactgactggaaataacctgcgcatacat
atttatatccatgttgatcccccttacttggtgcttgcccataagcccctggcttatcactgacgactgcgacg
acgagcagatctatttatacccaatggtgaccttggaaaccatggtataccgtaaatagaaaatttacatgg
taaccggtttccaaggtcaccattgggtataaatagatctgctcgtcgt
The sequence of Zophobas morio black wasting virus, strain “NJ2-molitor” follows:
(SEQ ID NO: 5)
actgcgacgacgagcagatctatttatacccaatggtgaccttggaaaccatggtataccatgtaaattttct
atttacggtataccatggtttccaaggtcaccattgggtataaatagatctgctcgtcgtcgcagtcgtcagt
gataagccaggggcttatgggcaagcaccaagtaagggagataaagtgaagtgtataaatagtgtttaac
atgtaagtacctcattctgttgttgtatttcgtgcagtgcagtaatactttggtttggtaagtaggtttaaacata
agaagatatatcaccctcagagcttgtatgacattactaagaagaaagtaagagaggtgtacaagaataat
tggaacgagacgaaggtactaccgaagactatccagagggaactactactagattggttaagatgtgac
gagagaattcctgagagtgatgacgatgtggaacgaatagtggcacgtatggaaaggggatgggaggc
attgaagccctttggtccgactacatttgtctacttgatgagattaccagatgaagtaccgccatttgcacat
gacaggaatcatatcatttgggacttctatttgtggtatgaacaagggcgagaaaagaaaatttgtgaagc
gtgttattccgggaagagtcgtttctaccgtccaggatctgctaatgaatggttagaaagaggatgggtattt
aaacgcgtagagaatcactccatgattgatggggacagattgttagaggatttgatatgggatgaagaca
actggtgcagtttatgtattgttgagccattatggatccatatatttgatgatgatgattgtttgtttgattacgac
tatcatttgaagagaaggcgtacatggagtgactcaagctctgaggacagtgatattgattattgtaaacat
actgttatgcaagggattcgtataaaccccactttgtataaatttattaatgaataaagcaagtgttatagaatt
gttgtttcattgcagatgaataatgacgaccacttttgggaatattttgacagtactctgggagatgcttccgg
acgagtgggcgaatcatccgggagtatggtggaagattatggatacgataccactggaccaacagtgca
agaatgcgatgttacagctaatagggcgttggagcaagaattacaaacaatggtcgaccggttcgttacc
cgccttgaaaaagaagattggcaagacagcggatactatgtgtcagatgtctttgcctgcgaatcaattgg
acgagctcagggattggctaagcgaatggctgaacgagcgggaaatttccgacgaggacttatccttata
tctatccacagcgatcaagatagcagtgcccatgtccacaccatccacagctgcgcctacgcaaacaggt
cgtgcaggtgctacttcaaagcgttccccgaagcgcaagaggacgctagacgacttcttcgaaagcctc
cacccatcgaaacgttcaagcgaagcgattgggaaaatatcacaaagtatttttgtacgaaagggagacg
agcaacgttcgctaaaatcaacggtgccatacaaagacttcctcttgaaattacaaatctatccgacattatc
ctatcaggccaagttgaaggaggatcacactcaggcctggagaactgccacgacccgcttgacaataac
gatgaacgaaaacgaagaattgaaccgaaaggtgatggaggtactcgaagtcgcaaaagaagaggtgt
tggcaacgcaggaggagatggaggaatcagaggggacacaggagtaattcttgatttaattaagaggta
tgctgtttgtccattatctgagatagtatatactcgcgagtacttggaaaatccccttgcagttaaaaggttag
atgatagggatgtaaagaacgctatagattgccatgctgctattattaatacttggaatagagaggattatgt
taagttttatgaggacccaaacactgttaaaatttggagtgcacgtaatattgactcggtggagttatattattt
aactgataatgaatctagagatattattaataaactgcttgattatcaatgtgggttagctaagaaacagtttgt
aacggatttgattaatgtaacagacatgaatattcccaagtgtaattgctttctaattgttagccctccaagtg
ctggcaagaactttctatgcgatgctattaaagattactatattaatgttggacaaatgcagaaccctaataa
atataataccttcgcgtatcaggattgtcacaatagacgacttcttatttggaatgaaccaaactacgaacct
agggaaacggagaatttgaagatgttatttggaggggataacttgtctgctaatgttaagtgtaaacctcaa
gctaatgtaaagcgtactccagtaatatgtatgagtaatgttgtacccagatttgctaatcatgaagcatttgc
tgacagagttattacatattattggaatgctgctccgttcttgaaagaggtaataaaaaaaccacgacctgat
tctgtaatgaatttattatatgaaatatacaattcataattatggttttacagttttaatcatttgcatcctgccatat
acattaggattatcatatactttagcaataggacgaccatcacttgcaaaatattgaagttgagatttggtatt
ggccgagtaacatccgcccttaatgtatgtgtaagggtcgacactctctgttgttagtacacaatcaacttcg
aagtagccttgtgcgtctagccaggaatttgcttgagtagtttcatcaatggtagttaacttgggaacagctc
ttatgccaacatttatagatggcatttgagtgtcatgtactgtctcaacgtttacctcctcgaatataccacctt
gttcaatagggactcgaagatagttctgttcctcatcgaagaatgtcgtatctgcgtggggaccctgtatata
cttgcgaggtgcattacgtacttgtgttggtgctgtagcttgtgaggaatcggttgttttcgctgccgtcactt
cattctttgttcccgcaggataactaccattatagttttgagtaatcaagttgtttggtactggtgcaaactgtg
gtaccagaggtgcgtagctaaaatcgtaatccatagcaactacatctgtgttgatacatgcacttgcatcga
attcttctataaagtttttgtatggtggaaaccccgccaatatcttagtaccagtggtagcccttgcatcattag
tgtaaatggttaaatagtcctgtagatatatctctgcgccagttgcatctgctggtggtttcttagcaaagtctg
cactgtcattatcgtaaccatacatagcaatcttgagaccatctcgatattggtaggatgtcaatgttgcgaa
tcccgtaggttgcatgggttcagtatcagaatatgtatactttctgtttgtagaacatataaaaggaatacttct
aatacccttagcaacttgcagaaatttgttttgattcagtgtagcattagccgtttgagtatctccagtttgaaa
tgcaactctagtattccatgttctaatacgtactgaagccgacttagcaaacgtcccaggatagttcttcattc
tgttatattctgctggactcatatagaagaataagtattcccatggaattgatgccattcctgtagttaatgccc
atcttgctggtctgtctccgctggccgcttcagccaatatgacgtttgcatttgctgaagtcagaaacctcca
tttctttctataagtttgttgaaacttctcaacatgaagtccaattggacgtaatatagctgtagctccgccttcc
ccactacctcctctaaccataccatctaagttgcctccagttccagggagactcgtccctgacttggatacc
ttactcggcggtccgccaccaccactacctcgcttcgatccaccgctagaacttgatacttgagacatttct
atgtccgccatgtttggttgtggtgcgttacttggtccggcgtctcttaagtcttgtctatctcttacttgttgtag
aaaatcgtctactccgtcttctgattgctgactgggttgttccgcctcctggggtgaccctaaaagatccaaa
tcaaagttgtcaatcgcatcagcctgagttaaaggtaggtcgtcatccccaggaggatcaaagtattcccc
acgtcttactctagctaggttccattgttccatagcataacgtctttgtccctcattcatactatcccaattagga
cgatcctgaggattcggacccagcggtctaactctaggcattgcttaccgtagtatacttttccacttaaacg
ttcgactgcgtgcttaactcccagcccgattcctcccactgccgcgtgtatcctagatataggatcctgacc
ctggattgcctcgtgtgcgaattgtgagattgcttctctatccgctgacagcacgtcgctatcgctcttagctt
gttgataatgtaagtcgtgaccctgcgcaataaggtcagctctgttggtcgcgggacgtattggatttccag
gtccgatattgttcgaaaacggtaaaactagtccgttccgtggtggttctccccttgctagattggcagaatc
tcttctcgcctgtgcttccctttgttcttgcttcttcaccttctttagtcccctgcgaggatttactagtctccagt
gttctggatatttagccttagtgtccttattccaaggatctccgtacggtttgatcttctcctgtcgctctttcaac
cccaaacgtctcaattcccaaggaacggctctgtagtaatcgtctcgattaccaaatattgctctcgataaa
gggttgttagggtcagctgctgctcttcccaattgcccttcgaatattggttgatattggctacctgctccaat
aaccgcacccccaacacgtggtgtattaggtactctcaatttactgtcacttccggcactggagatactact
attagatgagttaataggaatagatacgtatccacccctacgtcgcacccctgtacttgtactgggttgatta
aacacactctcttcactactgctgggggtaatagtgctcagctcgatgtactcggacatactgactggaaat
aacctgcgcatacatatttatatccatgttgatcccccttacttggtgcttgcccataagcccctggcttatca
ctgacgactgcgacgacgagcagatctatttatacccaatggtgaccttggaaaccatggtataccatgta
aattgaaaatttacatggtaacctggtttccaaggtcaccattgggtataaatagatctgctcgtcgtcgcag
tcgtc
The sequence of Zophobas morio black wasting virus, strain “OH-molitor” follows:
(SEQ ID NO: 6)
gtcgtcagtgataagccaggggcttatgggcaagcaccaagtaagggagataaagtgaagtgtataaat
agttttgaacatgtaagtagctcattctgttgttgtatttcgtgcagtgcagtaatactctgctttggtaagtagg
tttaaacataagaagatatatcaccctcagagcttgtatgacattactaagaagaaggtaagagaggtgtac
aagaataattggaacgagacgaaggtgctaccgaagactatccagagggaactattactagattggttaa
gatgtgacgagaagatttctgagagtgatgacgatgtggaacgaatagtggcacgtatggaaaggggat
gggaggcattgaagccctttggtccgaccacatttgtgtacttgatgagattaccagatgaagtaccgcca
tttgcacatgaaaggaatcatatcatttgggacttctacttgtggtatgaacaagggcgagaaaagaaaatt
tgtgaaccgtgttattccgggaagagtcgtttctaccgtccaggatctgctaatgaatggttggaaaaagga
tgggtatttaaacgcgtagagaatcactccatgattgatggggacagattgttagaggatttgatatgggat
gaagacaactggtgcagtttatgtattgttgagccattatggatccatatacttgatgatgatgattgtttgtttg
attatgactatcatttgaagagaaggcgtacatggagtgactcaagctctgaggacagtgatattgattattg
taaacatactgttatgcaagggattcgtataaaccccactttgtataaatttattaatgaataaagcaagtgtta
tagaattgttgtttcattacagatgaataatgacgaccacttttgggaatattttgacagtactctgggagatg
cttccggacgagtgggcgaatcatccgggagtatggtggaagattatggatacgataccactggaccaa
cagtgcaagaatgcgatgttgcagctaatagggcgttggagcaagaattacaaacaatggtcgaccggtt
cgttacccgccttgaaaaagaagattggcaagacagcggatactatgtgtcagatgtctttgcctgcgaat
caattggacgagctcaaggattggctaagcgaatggctgaacgagcgggaaatttccgacgaggactta
tccttatatctatccacagcgatcaagatagcagtgcccatgtccacaccatccacagctgcgcctacgca
aacaggtcgtgcaggtgctacttcaaagcgttccccgaagcgcaagaggacgctagacgacttcttcga
aagcctccacccatcgaaacgttcaagcgaagcgattgggaaaatatcacaaagtatttttgtacgaaag
ggagacgagcaacgttcgctaaaatcaacggtgccatacaaagacttcctcttgaaattacaaatctatc
cgacattatcctatcaggccaagttgaaggaggatcacactcaggcctggagaactgccacgacccgct
tgacaataacgatgaacgaaaacgaggaattgaaccgaaaggtgatggaggtactcgaagtcgcaaaa
gaagaggtgttggcaacgcaggaggagatggaggaatcagaggggacacaggagtaattcttgatttg
attaagaggtatgctgtttgtccattatctgagatagtatatactcgcgagtacttggaaaatcccattgcagt
taaaaggttagatgatagggatgtaaagaacgctatagattgccatgctgctattattaatacttggaataga
caggattatgttaagttttatgaggacccaaacactgttaaaatttggagtgcacgtaatgttgactcggtgg
agttatattatttaactgataatgaatctagagatattattaataaactgcttgattatcaatgtgggttagctaa
gaaacagtttgtaacggatttgattaatgtaacagacatgaatattcccaagtgtaattgctttctaattgttag
ccctccaagtgctggcaagaactttctatgcgatgctattaaagattactatattaatgttggacaaatgcag
aaccctaataaatataataccttcgcgtatcaggattgtcacaatagacgacttcttatttggaatgaaccaa
actacgaacctagggaaacggagaatttgaagatgttatttggaggggataacttgtctgctaatgttaagt
gtaaacctcaagcgaatgtaaagcgtactccagtaatatgtatgagtaatgttgtacccagatttgctaatca
tgaagcatttgctgacagagttattacatattattggaatgctgctcctttcttaaaagaggtaataaaaaagc
cacgacctgattctgtaatgaatttattatatgaaatatacaattcataattatggttttacagttttaatcatttgc
atcctgccatatacattaggattatcatatactttagcaataggacgaccatcacttgcaaaatattgaagttg
agatttggtattggccgagtaacatccgcccttaatgtatgtgtaagggtcgacactctctgttgttagtaca
caatcaacttcgaagtagccttgtgcgtctagccaggaattagcttgagtagtttcatcaatggtagttaactt
gggaacagctcttatgccaatatttatagatggcatttgagtgtcatgtactgtctcaacatttacctcctcga
atatacctccttgttcaatagggactcgaagatagttctgttcctcatcgaagaatgttgtatctgcattggga
ccctgtatatatttgcgaggtgcattacgtacttgtgttggtgctgtagcttgtgaggaatcggttgttttcgct
gccgtcacttcattctttgttcccgcaggataactaccattatagttttgagtaatcaagttgttcggtactggt
gcaaactgtggtaccagaggtgcgtagctaaaatcgtaatccatagcaactacatctgtgttgatacatgc
acttgcatcgaattcttctataaagtttttgtatggtggaaaccccgccaatatcttagtaccagtagtagccc
ttgcatcattagtgtaaatggttaaatagtcctgtagatatatctctgcgccagttgcatctgctggtggtttctt
agcaaagtctttattgtcgttatcgtaaccatacatagcagtcttgagaccatctcgatattggtatgatgtca
aggttgcgaatccagtaggttgcatgggttcagtatcagaatatgtatactttctgtttgtagagcatataaaa
ggaatacttctaatccccttagccacttgtaagaatttattctgatttagtgtagcattagccgtttgagtatctc
cagtttggaatgcaactcttgtgttccacgtcctaatacgtactgaagcggacttagcaaacgtcccaggat
agttcttcattcgattatattctgctggactcatataaaagaataagtattcccatggaattgatgccattcctgt
agttaatgcccatcttgctggtctttctccgctagctgcctcggccaatatgacgtttgcatttgctgaagtca
gaaacctccatttctttctataagtttgttgaaacttctcaacatgaagtccaattggacgtaatatagctgtag
ctccgccttccccactacctcctctaaccataccatctaagttgcctccagttccagggagactcgtccctg
acttggataccttactcggcggtccgccaccaccactgcctcgcttcgatccaccgctagaacttgatactt
gagacatttctatgtccgccatgtttggttgtggtgcgttacttggtccggcgtctcttaaatcttgtctatctct
tacttgttgtaaaaaatcgtctactccgtcttctgattgctgactgggttgttccgcctcctggggtgacccta
aaagatccaaatcaaagttatcaatcgcatcagcctgagttaaaggtaggtcgtcatccccaggaggatc
aaagtattccccacgtcttactctagctaggttccattgttccatagcataacgtctttgtccctcattcatacta
tcccaattaggacgatcctgaggattcggacccaacggtctaactctaggcattgcttaccgtagtatacttt
tccgcttaaatgttcgactgcgtgcttaattcccagcccgattcctcccactgccgcgtgtatcctagatata
ggatcctgaccctggattgcctcgtgtgcgaattgtgagattgcttctctatccgctgacagcacgtcgctat
cgctcttagcttgttgataatgtaagtcgtgaccctgcgcaataaggtcagctctgttggtcgcgggacgta
ttggatttccaggtccgatattgttcgaatacggtaaaacaagtccgttccgtggtggttctccccttgctag
attggcagaatctcttctcgcctgtgcttccctttgttcttgcttctttaccttctttagtcccctgcgaggattta
ctagtctccagtgttctggatatttagccttagtgtccttattccaaggatctccgtacggtttgatcttctcctg
tcgctctttcaaccccaaacgtctcaattcccaaggaacggctctgtagtaatcgtctcgattaccaaatatt
gctctcgataaagggttgttagggtcagctgctgctcttcccaattgcccttcgaatattggttgatattggct
acctgctccaaaagccgcacccccaacacgtggtgtattaggtactctcagtttactgtcacttccggcact
ggagatactactattagacgagttaataggaatagatacgtatccacccctacgtcgcacccctgtacttgt
actgggttgattaaacacactctcttcactactgctgggggtaatagtgctcagctcgatgtactcggacat
actgactggaaataacctgcgcatacatatttatatccatgttgatcccccttacttggtgcttgcccataagc
ccctggcttatcactgacgactgcgacgacgagcagatctatttatacccaatggtgaccttggaaacc
The sequence of Zophobas morio black wasting virus, strain “OR molitor” follows:
(SEQ ID NO: 7)
actgcgacgacgagcagatctatttatacccaatggtgaccttggaaaccagggtataccagtaaattgtata
tttacatggtataccatggtttccaaggtcaccattgggtataaatagatctgctcgtcgtcgcagtcgtcagtg
ataagccaggggcttatgggcaagcaccaagtaagggagataaagtgaagtgtataaatagtgtttaacat
gtaagtacctcattctgttgttgtatttcgtgcagtgcagtaatactttggtttggtaagtaggtttaaacataaga
agatatatcaccctcagagcttgtatgacattactaagaagaaggtaagagaggtgtacaagaataattgga
acgagacgaaggtgctaccgaagactatccagagggaactactactagattggttaagatgtgacgagaa
gatttctgagagtgatgacgatgtggaacgaatagtggcacgtatggaaaggggatgggaggcattgaag
ccctttggtccgaccgcatttgtgtacttgatgagattaccagatgaagtaccgccatttgcacatgaaagga
atcatatcatttgggacttttatttgtggtatgaacaagggcgagaaaagaaaatttgtgaaccgtgttattccg
ggaagagtcgtttctaccgtccaggatctgctaatgaatggttggaaaaaggatgggtatttaaacgcgtag
agaatcactccatgattgatggggacagattgttagaggatttgatatgggatgaagacaactggtgcagttt
gtgtattgttgagccattatggatccatatacttgatgatgatgattgtttgtttgattatgactatcatttgaagag
aagacgtacatggagtgactcaagctctgaggacagtgatattgattattgtaaacatactgttatgcaaggg
attcgtataaaccccactttgtataaatttattaatgaataaagcaagtgttatagaattgttgtttcattacagatg
aataatgacgaccacttttgggaatattttgacagtactctgggagatgcttccggacgagtgggcgaatcat
ccgggagtatggtggaagattatggatacgataccactggaccaacaatgcaagaatgcgatgttacagct
aatagggcgttggagcaagaattacaaacaatggtcgaccggttcgttacccgccttgaaaaagaagattg
gcaagacagcggatactatgtgtcagatgtctttgcctgcgaatcaattggacgagctcagggattggctaa
gcgaatggctgaacgagcgggaaatttccgacgaggacttatccttatatctatccacagcgatcaggatag
cagtgcccatgtccacaccatccacagctgcgcctacgcaaacaggtcgtgcaggtgctacttcaaagcgt
tccccgaagcgcaagaggacgctagacgacttcttcgaaagcctccacccatcgaaacgttcaagcgaag
cgattgggaaaatatcacaaagtatttttgtacgaaagggagacgagcaacgttcgctaaaatcaacggtgc
catacaaagacttcctcttgaaattacaaatctatccgacattatcctatcaggccaagttgaaggaggatcac
actcaggcctggagaactgccacgacccgcttgacaataacgatgaacgaaaacgaggaattgaaccga
aaggtgatggaggtactcgaagtcgcaaaagaagaggtgttggcaacgcaggaggagatggaggaatc
agaggggacacaggagtaattcttgatttaattaagaggtatgctgtttgtccattatctgagatagtatatact
cgcgagtacttggaaaatcccattgcagttaaaagattagatgatagggatgtaaagaacgctatagattgc
catgctgctattattaatacttggaatagacaggattatgttaagttttatgaggacccaaacactgttaaaattt
ggagtgcacgtaatgttgactcggtagagttatattatttaactgataatgaatctagagatattattaataaact
gcttgattatcaatgtgggttagctaagaaacagtttgtaacggatttgattaatgtaacagacatgaatattcc
caagtgtaattgctttctaattgttagccctccaagtgctggcaagaactttctatgcgatgctattaaagattac
tatattaatgttggacaaatgcagaaccctaataaatataatactttcgcgtatcaggattgtcacaatagacga
cttcttatttggaatgaaccaaactacgaacctagggaaacggagaatttgaagatgttatttggaggggata
acttgtctgctaatgttaagtgtaaacctcaagctaatgtaaagcgtactccagtaatatgtatgagtaatgttgt
acccagatttgctaatcatgaagcatttgctgacagagttattacatattattggaatgctgctcctttcttaaaa
gaggtaataaaaaagccacgtcctgattctgtaatgaatttattatatgaaatatacaattcataattatggtttta
cagttttaatcatttgcatcctgccatatacattaggattatcatatactttagcaataggacgaccatcacttgc
aaaatattgaagttgagatttggtattggccgagtaacatccgcccttaatgtatgtgtaagggtcgacactct
ctgttgttagtacacaatcaacttcgaagtagccttgtgcgtctagccaggaattagcttgagtagtttcatcaa
tggtagttaacttgggaacagctcttatgccaacatttatagatggcatttgagtatcatgtactgtctcaacattt
acctcctcgaatataccaccttgttcaatagggactcgaagatagttctgttcctcatcaaagaatgttgtatct
gcgtggggaccctgtatatatttgcgaggtgcattacgtacttgtgttggtgctgtagcttgtgaggaatcggt
tgttttcgctgccgtcacttcattcttcgttcccgcaggataactaccattataattttgagtaatcaagttgtttgg
tactggtgcaaactgcggtaccagaggtgcgtagctaaaatcgtaatccatagcaactacatctgtgttgata
catgcacttgcatcgaattcttctataaagtttttgtatggtggaaaccccgccaatatcttagtaccagtagtag
cccttgcatcattagtgtaaatggttaaatagtcctgtagatatatctctgcgccagttgcatctgctggtggttt
cttagcaaagtctttattatcgttatcgtaaccatacatagcagtcttgagaccatctcgatattggtaggatgtc
aatgttgcgaatcccgtaggttgcatgggttcagtatcagaatatgtatactttctgtttgtagaacatataaaag
gaatacttctaatacccttagcaacttgcagaaatttgttttgattcagtgtagcattagccgtttgagtatctcca
gtttgaaatgcaactctagtattccatgttctaatacgtactgaagccgacttagcaaacgtaccaggatagtt
cttcattcgattatattctgctggactcatataaaagaataagtattcccatggaattgatgccattcctgtcgtta
gcgcccatcttgctggtctttctccgctggccgcttcagccaatatgacgtttgcatttgctgaagtcagaaac
ctccatttctttctataagtttgttgaaacttctcaacatgaagtccaattggacgtaatatagctgtagctccgcc
ttccccactacctcctctaaccatgccatccaaattgcctccagttccagggagactcgtccctgacttggata
ccttactcggcggtccgccaccaccactgcctcgcttcgatccaccgctagaacttgatacttgagacatttc
tatgtctgccatgtttggttgtggtgcgttacttggtccggcgtctcttaaatcttgtctatctcttacttgttgtaaa
aaatcgtctactccgtcttctgattgctgactgggttgttccgcctcctggggtgaccctaaaagatccaaatc
aaagttatcaatcgcatcagcctgagttaaaggtaggtcgtcatccccaggaggatcaaagtattccccacg
tcttactctagctaggttccattgttccatagcataacgtctttgtccctcattcatactatcccaattaggacgat
cctgaggattcggacccaacggtctaactctaggcattgcttaccgtagtatacttttccacttaaatgttcgac
tgcgtgcttaactcccagcccgattcctcccactgccgcgtgtatcctagatataggatcctgaccctggatt
gcctcgtgtgcgaattgtgagattgcttctctatccgctgacagcacgtcgctatcgctcttagcttgttgataa
tgtaagtcgtgaccctgcgcaataaggtcagctctgttggtcgcgggacgtattggatttccaggtccgatat
tgttcgaatacggtaaaactagtccgttccgtggtggttctccccttgctagattggcagaatctcttctcgcct
gtgcttccctttgttcttgctttttcaccttctttagtcccctgcgaggatttactagtctccagtgttctggatattta
gccttagtgtccttattccaaggatctccgtacggtttgatcttctcctgtcgctctttcaaccccaaacgtctca
attcccaaggaacggctgcgtagtaatcgtcccgattaccaaatatcgctctcgataaagggttcctagggtc
agctgctgctcttcccaattgcccttcgaatattggttgatattggctacctgctccaataaccgcacccccaa
cacgtggtgtattaggtactctcaatttactgtcacttccggcactggagatactactattagacgagttaatag
gaatagatacgtatccacccctacgtcgcacccctgtacttgtactgggttgattaaacacactctcttcacta
ctgctgggggtaatagtgctcagctcgatgtactcggacatactgactggaaataacctgcgcatacatattt
atatccatgttgatcccccttacttggtgcttgcccataagcccctggcttatcactgacgactgcgacgacga
gcagatctatttatacccaatggtgaccttggaaaccatggtataccatgtaaatagtatatttacatggtaacc
atggtttccaaggtcaccattgggtataaatagatctgctcgtcgtcgcagtcgtc
The sequence of Zophobas morio black wasting virus, strain “NY3-molitor” follows:
(SEQ ID NO: 8)
agcagatctatttatacccaatggtgaccttggaaaccatggtataccatgtaaattttcaatttactggtatac
catggtttccaaggtcaccattgggtataaatagatctgctcgtcgtcgcagtcgtcagtgataagccagg
ggcttatgggcaagcaccaagtaagggagataaagtgaagtgtataaatagttttgaacatgtaagtagct
cattctgttgttgtattccgtgcagtgcagtaatactttggtttggtaagtaggtttaaacataagaagatatat
caccctcagagcttgtatgacattactaagaagaaggtaagagaggtgtacaagaataattggaacgaga
cgaaggtactaccgaggactatccagagggaactactactagattggttaagatgtgacgagaagatttct
gagagtgatgacgatgtggaacgaatagtggcacgtatggaaaggggatgggaggcattgaagcccttt
ggtccgaccacatttgtgtacttgatgagattaccagatgaagtaccgccatttgcacatgaaaggaatcat
atcatttgggacttctatttgtggtatgaacaagggcgagaaaagaaaatttgtgaaccgtgttattccggg
aagagtcgtttctaccgtccaggatctgctaatgaatggttggaaaaaggatgggtatttaaacgcgtaga
gaatcactccatgattgatggggacagattgttagaggatttgatatgggatgaagacaactggtgcagttt
atgtattgttgagccattatggatccatatatttgatgatgatgattgtttgtttgattatgactatcatttgaaga
gaaggcgtacatggagtgactcaagctctgaggacagtgatattgattattgtaaacatactgttatgcaag
ggattcgtataaaccccactttgtataaatttattaatgaataaagtaagtgttatagaattgttgtttcattaca
gatgaataatgacgaccacttttgggaatattttgacagtactctgggagatgcttccagacgagtgggcg
aatcatccgggagtatggtggaagattatggatacgataccactggaccaacagtgcaagaatgcgatgt
tgcagctaatagggcgttggagcaagaattacaaacaatggtcgaccggttcgttacccgccttgaaaaa
gaagattggcaagacagcggatactatgtgtcagatgtctttgcctgcgaatcaattggacgagctcaag
gattggctaagcgaatggctgaacgagcgggaaatttccgaagaggacttatcctcatatctattcacagc
gatcaagatggcggtgcccatatccacaccatccacagctgcgcctacgcaaacaggtcgtgcaggtgc
tacttcaaagcgttccccgaagcgcaagaggacgctagacgacttcttcgaaagcctccacccatcgaa
acgttcaagcgaagcgattgggaaaatatcacaaagtatttttgtacgaaagggagacgagcaacgttcg
ctaaaatcaacggtgccatacaaagacttcctcttgaaattacaaatctatccgacattatcctatcaggcca
agttgaaggaggatcacactcaggcctggagaactgccacgacccgcttgacaataacgatgaacgaa
aacgaggaattgaaccgaaaggtgatggaggtactcgaagtcgcaaaagaagaggtgttggcaacgca
ggaggagatggaggaatcagaggggacacaggagtaattcttgatttgattaagaggtatgctgtttgtcc
attatctgagatagtatatactcgcgagtacttggaaaatcccattgcagttaaaaggttagatgatagggat
gtaaagaacgctatagattgccatgctgctattattaatacttggaatagacaggattatgttaagttttatga
ggacccaaacactgttaaaatttggagtgcacgtaatgttgactcggtggagttatattatttaactgataatg
aatctagagatattattaataaactgcttgattatcaatgtgggttagctaagaaacagtttgtaacggatttga
ttaatgtaacagacatgaatattcccaagtgtaattgctttctaattgttagtcctccaagtgctggcaagaac
tttctatgcgatgctattaaagattactatattaatgttggacaaatgcagaaccctaataaatataataccttc
gcgtatcaggattgtcacaatagacgacttcttatttggaatgaaccaaactacgaacctagggaaacgga
gaatttgaagatgttatttggaggggataacttgtctgctaatgttaagtgtaaacctcaagctaatgtaaag
cgtactccagtaatatgtatgagtaatgttgtacccagatttgctaatcatgaagcatttgctgacagagttatt
acatattattggaatgctgctcctttcttaaaagaggtaataaaaaaaccacgacctgattctgtaatgaattt
attatatgaaatatacaattcataattatggttttacagttttaatcatttgcatcctgccatatacattaggattat
catatactttagcaataggacgaccatcacttgcaaaatattgaagttgagatttggtattggccgagtaaca
tccgcccttaatgtatgtgtaagggtcgacactctctgttgttagtacacaatcaacttcgaagtagccttgtg
cgtctagccaggaattagcttgagtagtttcatcaatggtagttaacttgggaacagctcttatgccaatattt
atagatggcatttgagtgtcatgtactgtctcaacatttacctcctcgaatataccaccttgttcaatagggac
tcgaagatagttctgttcctcatcaaagaatgttgtatctgcattgggaccctgtatatatttgcgaggtgcatt
acgtacttgtgttggtgctgtagcttgtgaggaatcggttgttttcgctgccgtcacttcattctttgttcccgc
aggataactaccattatagttttgagtaatcaagttgtttggtactggtgcaaactgcggtaccagaggtgc
gtagctaaaatcgtaatccatagcaactacatctgtgttgatacatgcacttgcatcgaattcttctataaagtt
tttgtatggtggaaaccccgccaatatcttagtaccagtagtagcccttgcatcattagtgtaaatggttaaat
agtcctgtagatatatctctgcgccagttgcatctgctggtggtttcttagcaaagtctttattgtcgttatcgta
accatacatagcagtcttgagaccatctcgatattggtaggatgtcaatgttgcgaatcccgtaggttgcat
gggttcagtatcagaatatgtatactttctgtttgtagaacatataaaaggaatacttctaatacccttagcaac
ttgcagaaatttgttttgattcagtgtagcattagctgtttgagtatctccagtttggaatgcaactcttgtgttcc
acgttctaatacgtactgaagcggacttagcgaacgtaccaggatagttcttcattctgttatattctgctgga
ctcatatagaagaataagtattcccatggaattgatgccattcctgtagttaatgcccatcttgctggtctttct
ccgctagctgcctcggccaatatgacgtttgcatttgctgaagtcagaaacctccatttctttcggtatgtttg
ttggaatttttcgacgtgaagtccaataggacgtaatatagctgtagctccgccttccccaccacctcctcta
accatgccatctaagttgcctccagttccagggagactcgtccctgacttggataccttactcggcggtcc
gccaccaccactacctcgcttcgatccaccgctagaacttgatacttgagacatttctatgtccgccatgttt
ggttgtggtgcgttacttggtccggcgtctcttaaatcttgtctatctcttacttgttgtaaaaaatcgtctactc
cgtcttctgattgctgactgggttgttccgcctcctggggtgaccctaaaagatccaaatcaaagttatcaat
cgcatcagcctgagttaaaggtaggtcgtcatccccaggaggatcaaagtattccccacgtcttactctag
ctaggttccattgttccatagcataacgtctttgtccctcattcatactatcccaattaggacgatcctgaggat
tcggacccaacggtctaactctaggcatctcttaccgtagtatacttttccgcttaaatgttcgactgcgtgct
taactcccagcccgattcctcccactgccgcgtgtaccctagatataggatcctgaccctggattgcctcgt
gtgcgaattgtgagattgcttctctatccgctgacagcacgtcgctatcgctcttagcttgttgataatgtaag
tcgtgaccctgcgcaataaggtcagctctgttggtcgcgggacgtattggatttccaggtccgatattgttc
gaatacggtaaaactagtccgttccgtggtggttctccccttgctagattggcagaatctcttctcgcctgtg
cttccctttcttcttgcttcttcaccttctttagtcccctgcgaggatttactagtctccagtgttctggatatttag
ccttagtgtccttattccaaggatctccgtacggtttgatcttctcctgtcgctctttcaaccccaaacgtctca
attcccaaggaacggctctgtagtaatcgtctcgattaccaaatattgctctcgataaagggttgttagggtc
agctgctgctcttcccaattgcccttcgaatattggttgatattggctacctgctccaaaagccgcaccccca
acacgtggtgtattaggtactctcaatttactgtcacttccggcactggagatactactattagacgagttaat
aggaatagatacgtatccacccctacgtcgcacccctgtacttgtactgggttgattaaacacactctcttca
ctactgctgggggtaatagtgctcagctcgatgtactcggacatactgactggaaatgacctgcgcataca
tatttatatccatgttgatcccccttacttggtgcttgcccataagcccctggcttatcactgacgactgcgac
gacgagcagatctatttatacccaatggtgaccttggaaaccatggtataccatgtaaattttctatttacagg
tataccatggtttccaaggtcaccattgggtataaatagatctg
The sequence of Zophobas morio black wasting virus, strain “PA-molitor” follows:
(SEQ ID NO: 9)
agcagatctatttatacccaatggtgaccttggaaaccggttaccatgtaaattttctatttacggtataccat
ggtttccaaggtcaccattgggtataaatagatctgctcgtcgtcgcagtcgtcagtgataagccaggggc
ttatgggcaagcaccaagtaagggagataaagtgaagtgtataaatagtgttcaacatgtaagtagctcatt
ctgttgttgtatttcatgcagtgcagtaatactttgcattggtaagtaggtttaaacataagaagatatatcacc
ctcagagcttgtatgacattactaagaagaaggtaagagaggtgtacaagaataattggaacgagacgaa
ggtgctaccgaagactatccagagggaactactactagattggttaagatgtgacgagaagatttctgaga
gtgacgacgatgtggaacgaatagtggcacgtatggaaaggggatgggaggcattgaagccctttggtc
cgaccacatttgtgtacttgatgagattaccagatgaagtaccgccatttgcacatgaaaggaatcatatca
tttgggacttctatttgtggtatgaacaagggcgagaaaagaaaatttgtgaaccgtgttattccgggaaga
gtcgtttctaccgtccaggatctgctaatgaatggttggaaaaaggatgggtatttaaacgcgtagagaatc
actccatgattgatggggacagattgttagaggatttgatatgggatgaagacaactggtgcagtttatgtat
tgttgagccattatggatccatatacttgatgatgatgattgtttgtttgattatgactatcatttcaagagaaga
cgtacatggagtgactcaagctctgaggacagtgatattgattattgtaaacatactgttatgcaagggattc
gtataaaccccactttgtataaatttattaatgaataaagcaagtgttatagaattgttgtttcattacagatgaa
taatgacgaccacttttgggaatattttgacagtactctgggagatgcttccggacgagtgggcgaatcatc
caggagtatggtggaagattatggatacgataccactggaccaacaatgcaagaatgcgatgttacagct
aatagggcgttggagcaagaattacaaacaatggtcgaccggttcgttacccgccttgaaaaagaagatt
ggcaagacagcggatactatgtgtcagatgtctttgcctgcgaatcaattggacgagctcagggattggct
aagcgaatggctgaacgagcgggaaatttccgaagaggacttatcctcatatctattcacagcgatcaag
atggcggtgcccatatccacaccatccacagctgcgcctacgcaaacaggtcgtgcaggtgctacttcaa
agcgttccccgaagcgcaagaggacgctagacgacttcttcgaaagcctccacccatcgaaacgttcaa
gcgaagcgattgggaaaatatcacaaagtatttttgtacgaaagggagacgagcaacgttcgctaaaatc
aacggtgccatacaaagacttcctcttgaaattacaaatctatccgacattatcctatcaggccaagttgaa
ggaggatcacactcaggcctggagaactgccacgacccgcttgacaataacgatgaacgaaaacgag
gaattgaaccgaaaggtgatggaggtactcgaagtcgcaaaagaagaggtgttggcaacgcaggagg
agatggaggaatcagaggggacacaggagtaattcttgatttaattaagaggtatgctgtttgtccattatct
gagatagtatatactcgcgagtacttggaaaatcccattgcagttaaaaggttagatgatagggatgtaaa
gaacgctatagattgccatgctgctattattaatacttggaatagacaggattatgttaagttttatgaggacc
caaacactgttaaaatttggagtgcacgtaatgttgactcggtggagttatattatttaactgataatgaatcta
gagatattattaataaactgcttgattatcaatgtgggttagctaagaaacagtttgtaacggatttgattaatg
taacagacatgaatattcccaagtgtaattgctttctaattgttagccctccaagtgctggcaagaactttctat
gcgatgctattaaagattactatattaatgttggacaaatgcagaaccctaataaatataataccttcgcgtat
caggattgtcacaatagacgacttcttatttggaatgaacccaactacgaacctagggaaacggagaattt
gaagatgttatttggaggggataacttgtctgctaatgttaagtgtaaacctcaagctaatgtaaagcgtact
ccagtaatatgtatgagtaatgttgtacccagatttgctaatcatgaagcatttgctgacagagttattacatat
tattggaatgctgctcctttcttaaaagaggtaataaaaaaaccacgacctgattctgtaatgaatttattatat
gaaatatacaattcataattatggtttcacagttttaatcatttgcatcctgccatatacattaggattatcatata
ctttagcaataggacgaccatcacttgcaaaatattgaagttgagatttggtattggccgagtaacatccgc
ccttaatgtatgtgtaagggtcgacactctctgttgttagtacacaatccacttcgaagtagccttgtgcgtct
agccaggaattagcttgagtagtttcatcaatggtagttaacttgggaacagctcttatgccaatatttataga
tggcatttgagtgtcatgtactgtttcaacatttacctcctcgaatataccaccttgttcaatagggactcgaa
gatagttctgttcctcatcgaagaatgttgtatctgcgtggggaccctgtatatatttgcgaggtgcattacgt
acttgtgttggtgctgtagcttgtgaggaatcggttgttttcgctgccgtcacttcattctttgttcccgcagga
taactaccattataattttgagtaatcaagttgtttggtactggtgcaaactgtggtaccagaggtgcgtagct
aaaatcgtaatccatagcaactacatctgtgttgatacatgcacttgcatcgaattcttctataaagtttttgtat
ggtggaaaccccgccaatatcttagtaccagtagtagcccttgcatcattagtgtaaatggttaaatagtcct
gtagatatatctctgcgccagttgcatctgctggtggtttcttagcaaagtctgcactgtcattatcgtaacca
tacatagcaatcttgagaccatctcgatattggtaggatgtcaaggttgcgaatccagtaggttgcatgggt
tcagtatcagaatatgtatactttctgtttgtagagcatataaaaggaatacttctaatccccttagccacttgt
aggaatttattctgatttagtgtagcattagatgtttgagtatctccagtttggaatgcaactcttgtgttccacg
tcctaatacgtactgaagcggacttagcaaacgtaccaggatagttcttcattctgttatattctgctggactc
atataaaagaataagtattcccatggaattgatgccattcctgtagttaatgcccatcttgctggtctttctccg
ctggccgcttcagccaatatgacgtttgcatttgctgaagtcagaaacctccatttctttctataagtttgttga
aacttctcaacatgaagtccaattggacgtaatatagctgtagctccgccttccccactacctcctctaacca
taccatccaaattgcctcctgttcctggtaaacttgtcccagacttggataccttactcggcggtccgctacc
atcactacctcgcttggatccactactagagctagatacagacatttctacgtccgccatgtttggttgtggt
gcgttacttggtccggcgtctcttaaatcttgtctatctcttacttgctgtaaaaaatcgtctactccgtcttctg
attgctgactgggttgttccgcctcctggggtgaccctaaaagatccaaatcaaagttatcaatcgcatcag
cctgagttaaaggtaggtcgtcatccccaggaggatcaaagtattccccacgtcttactctagctaggttcc
attgttccatagcataacgtctttgtccctcattcatactatcccaattaggacgatcctgaggattcggaccc
aacggtctaactctaggcattgcttaccgtagtatacttttccgcttaaatgttcgactgcgtgcttaactccc
agcccgattcctcccactgccgcgtgtatcctagatataggatcctgaccctggattgcctcgtgtgcgaat
tgtgagattgcttctctatccgctgacagcacgtcgctatcgctcttagcttgttgataatgtaagtcgtgacc
ctgcgcaataaggtcagctctgttggtcgcgggacgtattggatttccaggtccgatattgttcgaaaacg
gtaaaactagtccgttccgtggtggttctccccttgctagattggcagaatctcttctcgcctgtgcttcccttt
gttcttgcttcttcgccttctttagtcccctgcgaggatttactagtctccagtgttctggatatttagccttagtg
tccttattccaaggatctccgtacggtttgatcttctcctgtcgctctttcaaccccaaacgtctcaattcccaa
ggaacggctgcgtagtaatcgtctcgattaccaaatattgctctcgataaagggttcctagggtcagctgct
gctcttcccaattgcccttcgaatattggttgatattggctacctgctccaataaccgcacccccaacacgtg
gtgtattaggtactctcaatttactgtcacttccggcactggagatactactattagacgagttaataggaata
gatacgtatccacccctacgtcgcacccctgtacttgtactgggttgattaaacacactctcttcactactgct
gggggtaatagtgctcagctcgatgtactcggacatactgactggaaatgacctgcgcatacatatttatat
ccatgttgatcccccttacttggtgcttgcccataagcccctggcttatcactgacgactgcgacgacgag
cagatctatttatacccaatggtgaccttggaaaccggttaccatgtaaattttctatttacggtataccatgg
tttcca

### Example 7: Engineered Vaccine Virus Strain

The engineered vaccine strain which forms an embodiment of this invention is derived from a non-pathogenic virus derived from a related species of beetle (Tenebrio molitor) and is referred to herein as “NJ2-molitor.” differs from the natural strain in that one or more mutations are introduced that reduce the length or expression level of the NS3 protein. In one embodiment, the start codon of the NS3 gene is mutated to a codon other than the canonical ATG start codon. In another embodiment, the NS3 gene was truncated at the amino-terminus by use of a second start codon.

The sequence of engineered strain 1 is as follows:
(SEQ ID NO: 10)
GACGACCAGATCTATTTATACCCAATGGTGACCTTGGAAACCTGGTTACCATGT
AAATTTTCTATTTACTGGTATACCATGGTTTCCAAGGTCACCATTGGGTATAAATA
GATCTGGTCGTCGTCGCAGTCGTCAGTGATAAGCCAGGGGCTTATGGGCAAGCA
CCAAGTAAGGGGGATAAAGTGAAGTGTATAAATAGTGTTCAACATGTAAGTAGC
TCATTCTGTTGTTGTATTTCGTGCAGTGCAGTAATACTTTGGTTTGGTAAGTAGGT
TTAAACATAAGAAGATATATCACCCTCAGAGCTTGTATGACATTACTAAGAAGA
AGGTAAGAGAGGTGTACAAGAATAATTGGAACTTGCTGGTACTACCGAATCGTA
TCCAGAGGGAACTACTACTAGATTGGTTAAGATGTGACGAGACAATTCCGGAGA
GTGATGACGATGTGGAACGAATAGTGGCACGTATGGAAAGGGGATGGGAGGCAT
TGAAGCCCTTTGGTTCGACTACATTTGTGTACTTGATGAGATTACCAGATGAAGT
ACCGCCATTTGCACATGAAAGGAATCATATCATTTGGGACTTTTACTTGTGGTAT
GAACAAGGGCGAGAAAAGAAAATTTGTGAACCGTGTTATTCCGGGAAGAGTCGT
TTCTACTGTCCAGGATCTGCTAATGAATGGTTAGAAAAAGGATGGGTATTTAAAC
GCGTAGAGAATCACTCCATGATTGATGGGGACAGATTGTTACAGGAGTTGATAT
GGGATGAAGACAACTGGTGCAGTTTATGTCTTGTTGAGCCATTATGGATCCATAT
ATTGGATGATGATGATTGTTTGTTTGATTATGACTATCATTTGAAGAGAAGGCGT
ACATGGAGTGACTCAAGCTCTGAGGACAGTGATATTGATTATTGTAAACATACTG
TTATGCAAGGGATTCGTATGAACCCCACTTTGTATAAATTTATTAATGAATAAAG
CAAGTGTTATAGAATTGTTGTTTCATTACAGATGAATAATGACGACCACTTTTGG
GAATATTTTGACAGTACTCTGGGAGATGCTTCCGGACGAGTGGGCGAATCATCCG
GGAGTATGGTGGAAGATTATGGATACGATACCACTGGACCAACAATGCAAGAAT
GCGATGTTACAGCTAATAGGGCGTTGGAGCAAGAATTACAAACAATGGTCGACC
GGTTCGTTACCCGCCTTGAAAAAGAAGATTGGCAAGACAGCGGATACTATGTGT
CAGATGTCTTTGCCTGCGAATCAATTGGACGAGCTCAGGGATTGGCTAAGCGAAT
GGCTGAACGAGCGGGAAATTTCCGACGAGGACTTATCCTTATATCTATCCACAGC
GATCAGGATGGCAGTGCCCATGTCCACACCATCCACAGCTGCGCCTACGCAAAC
AGGGCGTGCAGGTGCTACTTCAAAGCGTTCCCCGAAGCGCAAGAGGACGCTAGA
CGACTTCTTCGAAAGCCTCCACCCATCGAAACGTTCAAGCGAAGCGATTGGGAA
AATATCACAAAGTATTTTTGTACGAAAGGGAGACGAGCAACGTTCGCTAAAATC
AACGGTGCCATACAAAGACTTCCTCTTGAAATTACAAATCTATCCGACATTATCC
TATCAGGCCAAGTTGAAGGAGGATCACACTCAGGCCTGGAGAACTGCCACGACC
CGCTTGACAATAACAATGAACGAAAACGAGGAATTGAACCGAAAGGTGATGGA
GGTACTCGAAGTCGCAAAAGAAGAGGTGTTGGCAACGCAGGAGGAGATGGAGG
AATCAGAGGGGACACAGGAGTAATTCTTGATTTAATTAAGAGGTATGCTGTTTGT
CCATTATCTGAGATAGTATATACTCGCGAGTACTTGGAAAATCGTATTGCAGTTA
AAAGGTTAGATGATAGGGATGTAAAGAACGCTATAGATTGCCATGCTGCTATTAT
TAATACTTGGAATAGAGAGGATTATGTTAAGTTCTATGAGGACCCAAACACTGTT
AAAATTTGGAGTGCACGTAGTATTGACTTGGTGGAATTATATTATTTAACTGATA
ATGAATCGAGGGATATTATTAATAAACTGCTGGATTACCAATGTGGGTTAGCTAA
GAAACAGTTTGTAACAGATTTGATTAATGTAACAGACATGAATATTCCCAAGTGT
AATTGCTTTCTAATTGTTAGCCCTCCAAGTGCTGGCAAGAACTTTCTATGCGATGC
TATTAAAGATTACTATCTTAATGTTGGACAAATGCAGAACCCTAATAAATATAAT
ACCTTCGCGTATCAGGATTGTCACAATAGACGACTTCTTATTTGGAATGAACCAA
ACTACGAACCTAGAGAAACGGAGAATTTAAAGATGTTATTTGGAGGGGATAACT
TGTCTGCTAATGTTAAGTGTAAACCTCAAGCTAATGTAAAGCGTACTCCAGTAAT
ATGTATGAGTAATGTTGTACCCAGATTTGCTAATCATGAAGCATTTGCTGACAGA
GTTATTACATATTATTGGAATGCTGCTCCGTTCTTGAAAGAGGTAATAAAAAAAC
CACGACCTGATTCTGTAATGAATTTATTATATGAAATATACAATTCATAATTATG
GTTTTACAGTTTTAATCATTTGCATCCTGCCATATACATTAGGATTATCATATACT
TTAGCAATAGGACGACCATCATTTGCAAAATATTGAAGTTGAGATTTGGTATTGG
CCGAGTAACATCCGCCCTTAATGTATGTGTAAGGGTCGACACTCTCTGTTGTTAG
TACACAATCAACTTCGAAGTAGCCTTGTGCGTCTAGCCAGGAATTAGCTTGAGTA
GTTTCATCAATGGTAGTTAACTTGGGAACAGCTCTTATGCCAACATTTATAGATG
GCATTTGAGTGTCATGTACTGTCTCAACATTTACCTCCTCGAATATACCTCCTTGT
TCAATAGGGACTCGAAGATAGTTCTGTTCCTCATCAAAGAATGTTGTATCTGCAT
TGGGACCCTGTATATATTTGCGAGGTGCATTACGTACTTGTGTTGGTGGTGTAGC
TTGTGAGGAATCGGTTGTTTTCGCTGCCGTCACTTCATTCTTTGTTCCCGCAGGAT
AACTAGCATTATAGTTTTGAGTGATCAAGTTGTTTGGTACTGGTGCAAACTGTGG
TACCAGAGGTGCGTAGCTAAAATCGTAATCCATGGCAACTACATCTGTGTTGATA
CATGCACTTGCATCGAATTCTTCTATAAAGTTTTTGTATGGTGGAAACCCCGCCA
ATATCTTAGTACCAGTAGTAGCCCTTGCATCATTAGTGTAAATGGTTAAATAGTC
CTGTAGATATATCTCTGCGCCAGTTGCATCTGCTGGTGGTTTCTTAGCAAAGTCTG
GACTGTCATTATCGTAACCATACATAGCAATCTTGAGACCATCTCGATATTCGTA
GGATGTCAATGTTGCGAATCCCGTAGGTTGCATGGGTTCAGTATCAGAATATGTA
TACTTTCTGTTTGTAGAGCATATAAAAGGAATACTTCTAATACCCTTAGCAACTT
GCAGAAATTTGTTTTGATTCAGTGTAGCATTAGCCGTTTGAGTATCTCCAGTTTGA
AATGCAACTCTAGTATTCCATGTTCTAATACGTACTGAAGCCGACTTAGCGAACG
TCCCAGGATAGTTCTTCATTCGATTATATTCTGCTGGACTCATATAAAAGAATAA
GTATTCCCATGGAATTGATGCCATTCCTGTGGTTAATGCCCATCTTGCTGGTCTTT
CTCCGCTAGCTGCCTCGGCCAAGATGACGTTGGCATTTGCTGAAGTTAGGAATCT
CCATTTCTTTCTATAAGTTTGTTGAAACTTTTCAACATGAAGTCCAATTGGACGTA
ATATAGCTGTAGCTCCGCCTTCCCCACTACCTCCTCTAGCCATACCATCCAAATTG
CCTCCTGTTCCTGGTAAACTTGTCCCAGACTTGGATACCTTACTCGGCGGTCCGCT
ACCATCACTACCTCGCTTGGATCCACTACTAGAGCTAGATACTTGAGCCATTTCT
ACGTCCGCCATGTTTGGTTGTGGTGCGTTACTTGGTCCGGCGTCTCTTAAATCTTG
TCTATCTCTTACTTGTTGTAAAAAATCGTCTACTCCGTCTTCTGATTGCTGACTGG
GTTGTTCCGCCTCCTGGGGTGACCCTAAAAGATCCAAATCAAAGTTATCAATTGC
ATCAGCCTGAGTTAAAGGTAGGTCGTCATCCCCAGGAGGATCAAAATATTCTCCA
CGTCTTACTCTAGCTAGGTTCCATTGTTCCATAGCATAGCGTCTTTGTCCATCATT
CATACTATCCCAATTAGGACGATCTTGGGGATTCGGACCCAGCGGTCTAACTCTA
GGCATTGCTTACCGTAATATACTTTTCCACTTAGACGTTCGACTGCGTGCTTAACT
CCCAACCCGACTCCTCCCACTGCCGCGTGTATCCTAGATATAGGATCCTGACCCT
GGATTGCCTCGTGTGCGAATTGTGAGATTGCTTCTCTATCCGCTGACAGCACGTC
GCTATCGCTCTTAGCTTGTTGATAATGTAAGTCGTGACCCTGCGCAATAAGGTCA
GCTCTGTTGGTCGCGGGACGTACTGGATTTCCAGGTCCGATATTGTTCGAAAACG
GTAAAACTAGTCCGTTCCGTGGTGGTTCTCCCCTTGCTAGATTGGCAGAATCTCTT
CTCGCCTGCGCTTCCCTTTGTTCTTGCTTCTTCGCCTTGTTTAGTCCCCTGCGAGGA
TTTACTAGTCTCCAGTGTTCTGGATATTTAGCCTTAGTGTCCTTATTCCAAGGATC
TCCGTACGGTTTGATCTTCTCCTGTCGCTCTTTCAACCCCAAACGTCTCAATTCCC
AAGGAACGGCTGCGTAGTAATCGTCTCGATTACCAAATATTGCTCTCGATAAAGG
GTTGTTAGGGTCAGCTGCTGCTCTTCCCAATTGCCCTTCGAATATTGGTTGATATT
GGCTACCTGCTCCAATAACCGCACCCCCAACACGTGGTGTATTAGGTACCTTCAA
TTTACTGTCACTTCCGGCACTGGAGATACTACTATTAGACGAGTTAATAGGAATA
GATACGTATCCACCCCTACGTCGCACCCCTGTACTTGTATTGGGTTGATTAAACA
CACTCTCTTCACTACTGCTGGGGGTAATAGTGCTCAGCTCGATGTACTCGGACAT
ACTGACTGGAAATAACCTGCGCATACATATTTATATCCATGTTGATCCCCCTTACT
TGGTGCTTGCCCATAAGCCCCTGGCTTATCACTGACGACTGCGACGACGACCAGA
TCTATTTATACCCAATGGTGACCTTGGAAACCGGTTACCATGTAAATTTTCTATTT
ACGGTATACCATGGTTTCCAAGGTCACCATTGGGTATAAATAGATCTGGTCGTCG
TC

### Example 8. Experimental Inoculation of Z. morio Larvae

For direct fat body injections, a ZmBWV virus stock with a known titer, diluted to the desired concentration, was used. Approx. 10 μl of these was injected into each larvae, using a 1 ml insulin syringe with a delicate needle. The fat bodies of the first five abdominal segments were targeted by the needle. Mock-infected individuals were injected the same way with 1×PBS. To inoculate the larvae with contaminated food, we used deceased blackened individuals. In case of Z. morio larval carcasses, one carcass was provided for every 10 individuals, while one carcass was provided per every three individuals in case the deceased T. molitor larvae. In order to infect the Z. morio larvae by dripping virus suspension on their cuticules, the ZmBWV virus stocks were diluted to the desired concentration with 1×PBS and a 100 μl of these were dripped dropwise on the healthy larvae for every 10 individuals.

### Example 9. Methods of Vaccine Production and Use

Frozen corpses of larvae (T. molitor) are prepared as follows: We aim to test two methods of preparation for this product. In one case, healthy two-week old Z. morio larvae is exposed to the vaccine strain by feeding. Briefly, a virus suspension in the concentration of 10¹³ to 10¹⁵ gc/mL of the ZmBWV vaccine strain is dripped on carrot slices, which the animals eagerly consume. The exposed larval population ts monitored on a weekly basis for viral concentration and the larvae are placed on dry ice when the viral yield reaches 10¹⁴ to 10¹⁵ gc/mL, established by using a pool of five randomly selected larvae. In another method, the same virus suspension in 1×PBS is dripped on the cuticle of the larvae, followed by the same screening and freezing procedure as detailed above. In another method, a gelling agent with excipients and flavorants, such as gelatin with glucose syrup, is mixed with heated water. The virus suspension is added before the mixture sets into a solid gel. Pieces of the gel are fed to Z. morio larval colonies.

For vaccination, this product is fed to Z. morio larval colonies or otherwise administered, for example by spraying or dipping. To obtain these suspensions, approximately 4-week old Z. morio larvae are individually injected by 10⁷ gc of the ZmBWV vaccine strain. The larval colony is regularly subjected to virus titer quantification, to establish that the highest yield of the viral vaccine strain was achieved. When the viral yield reaches 10¹⁴ to 10¹⁵ gc/mL, the vaccine strain is purified directly from these larvae, hoping to yield a suspension of 10¹⁴ to 10¹⁵ gc/mL concentration. Approximately 50 individuals yield 2 mL suspension at the desired concentration. As this concentration is acutely toxic to utilize directly as a vaccine, the obtained suspension is diluted with 1×PBS by a 1000- to 10,000-fold.

The optimal vaccination timepoint and of the necessity of boosters can be determined by the person of skill, however vaccination can start as soon as possible as the virulent ZmBWV strains are capable of successfully infecting larvae of only a couple of days of age. Preliminary data indicates that the vaccine strain is maintained by the larvae for at least four months post inoculation, which suggests that frequent optional boosters may not be required. As for quantities, studies show that acceptable vaccine strain titers can be achieved by feeding one larva per every 30 individuals to be vaccinated at the age of two weeks. This quantity may be doubled every two weeks, e.g. for 4-week old larvae 15 individuals may be vaccinated per larva.

To produce a vaccine product in solution, the larvae can be vaccinated in batches, as they need to be devoid of substrate. The vaccine suspension is directly sprayed on their cuticle, followed by a thorough manual mixing of the batch. The vaccinated batch of larvae are left without adding fresh substrate for 24 hours, after which they are housed in their regular substrate of wheat bran or oatmeals. If administered by injection, each larva is held firmly in one hand, while injected into the abdominal fat bodies with a delicate insulin needle. One should be cautious to avoid puncturing the posterior section of the midgut. Each animal should be administered approximately 10 μL of vaccine suspension. If administered by feeding dead larvae, the vaccine strain containing frozen larvae is thawed at room temperature and warmed to at least 20° C. Following this, these larvae are mixed evenly among the individuals to be vaccinated. Feeding success may be increased by intermittent stirring of the bin, to ensure that each individual gets access to the vaccine carrying larvae. If administered by feeding a gel, the gel is placed in the bin with larvae and the bin may be intermittently stirred to ensure that each individual gets access to the gel.

### Example 10: Vaccine Trial

Groups of larvae were reared and injected using syringes with either saline (control) or the protective vaccine virus “NJ2-molitor” (SEQ ID NO:5) at a dose of 10⁹ genome copies (gc) into the fat body. After three weeks (21 days post-inoculation), the larvae were injected with either saline (control) or the pathogenic virus that causes Zophobas morio black wasting disease (ZmBWV strain UT-morio at 10⁷ gc). The strain UT-morio previously was shown to be infectious by dripping/spraying, and by adding dead infected larvae to be eaten by the live larvae.

Control larvae that were inoculated exclusively by the strain UT-morio with no prior inoculation with vaccine, reached 100% mortality 21 d.p.i. The double-inoculated larvae (larvae injected with the NJ2-molitor strain prior to ZmBWV exposure), exhibited a 30% survival at the termination of the experiment (32 d.p.i.). This group also showed a seven-day-long delay in the onset of the first symptoms, compared to the single, strain UT-morio-inoculated treatment group. These data show that the vaccine is effective in reducing larval death and morbidity in captive colonies of the superworm (Zophobas morio). See FIG. 4.

### REFERENCES

All references listed below and throughout the specification are hereby incorporated by reference in their entirety.
1. Adams et al., “PHENIX: a comprehensive Python-based system for macromolecular structure solution.” Acta Crystallogr D Biol Crystallogr 66(Pt 2): 213-221, 2010.
2. Auguste et al., “A newly isolated reovirus has the simplest genomic and structural organization of any reovirus.” J Virol 89(1): 676-687, 2015.
3. Bakonyi et al. Identification of a novel densovirus in the darkling beetle Zophobas morio. in Abstracts of the 17th International Congress of the Hungarian Society for Microbiology. 2015.
4. Baquerizo-Audiot, et al., Structure and expression strategy of the genome of Culex pipiens densovirus, a mosquito densovirus with an ambisense organization. J Virol, 83(13): p. 6863-73, 2009.
5. Bochow et al., First complete genome of an Ambidensovirus; Cherax quadricarinatus densovirus, from freshwater crayfish Cherax quadricarinatus. Mar Genomics. 24 Pt 3: p. 305-12, 2015.
6. Bonami et al., Characterization of hepatopancreatic parvo-like virus, a second unusual parvovirus 35 pathogenic for penaeid shrimps. J Gen Virol. 76 (Pt 4): p. 813-7, 1995.
7. Cotmore et al., “ICTV Virus Taxonomy Profile: Parvoviridae.” J Gen Virol 100(3): 367-368, 2019.
8. Cotmore and Tattersall, Parvoviruses: Small Does Not Mean Simple. Annu Rev Virol. 1(1): p. 517-37, 2014.
9. Cotmore et al., “The family Parvoviridae.” Arch Virol 159(5): 1239-1247, 2014.
10. Duffield et al., “Active and Covert Infections of Cricket Iridovirus and Acheta domesticus Densovirus in Reared Gryllodes sigillatus Crickets.” Front Microbiol 12: 780796, 2021.
11. Emsley et al., “Features and development of Coot.” Acta Crystallogr D Biol Crystallogr 66(Pt 4): 486-501, 2010.
12. Farr et al., “Parvoviral virions deploy a capsid-tethered lipolytic enzyme to breach the endosomal membrane during cell entry.” Proc Natl Acad Sci USA 102(47): 17148-17153, 2005.
13. Fediere et al., Genome organization of Casphalia extranea densovirus, a new iteravirus. Virology. 23 292(2): p. 299-308, 2002.
14. Flegel, “Historic emergence, impact and current status of shrimp pathogens in Asia.” J Invertebr Pathol 110(2): 166-173, 2012.
15. Francois et al., A New Prevalent Densovirus Discovered in Acari. Insight from Metagenomics in Viral Communities Associated with Two-Spotted Mite (Tetranychus urticae) Populations. Viruses, 2019. 11(3).
16. Grant et al., “cisTEM, user-friendly software for single-particle image processing.” Elife 7, 2019.
17. Gudenkauf, B. M., et al., Discovery of urchin-associated densoviruses (family Parvoviridae) in coastal waters of the Big Island, Hawaii. J Gen Virol, 2014. 95(Pt 3): p. 652-8.
18. Guo and Jiang, Single particle cryo-electron microscopy and 3-D reconstruction of viruses. Methods Mol Biol. 1117: p. 401-43, 2014.
19. Halder et al., “Structural characterization of H-1 parvovirus: comparison of infectious virions to empty capsids.” J Virol 87(9): 5128-5140, 2013.
20. Halder, et al., “Structure of neurotropic adeno-associated virus AAVrh.8.” J Struct Biol 192(1): 21-36, 2015.
21. Hewson, I., et al., Densovirus associated with sea-star wasting disease and mass mortality. Proc Natl Acad Sci USA, 2014. 111(48): p. 17278-83.
22. Jackson, E. W., et al., A Highly Prevalent and Pervasive Densovirus Discovered among Sea Stars from the North American Atlantic Coast. Appl Environ Microbiol, 86(6), 2020.
23. Jose et al., High-Resolution Structural Characterization of a New Adeno-associated Virus Serotype 5 Antibody Epitope toward Engineering Antibody-Resistant Recombinant Gene Delivery Vectors. J Virol. 93(1), 2019.
24. Holm, “DALI and the persistence of protein shape.” Protein Sci. 2019.
25a. Huang et al., “Characterization of the Adeno-Associated Virus 1 and 6 Sialic Acid Binding Site.” J Virol 90(11): 5219-5230, 2016.25b. Jamali et al., “ModelAngelo: automated model building in Cryo-EM maps.” bioRxiv 2022 arXiv:2210.00006.
26. Kailasan et al., Structure of an enteric pathogen, bovine parvovirus. J Virol. 89(5): p. 2603-14, 2015.
27. Kang et al., Densoviruses in oyster Crassostrea ariakensis. Arch Virol. 162(7): p. 2153-2157, 2017.
28. Kapelinskaya et al., “Expression strategy of densonucleosis virus from the German cockroach, Blattella germanica.” J Virol 85(22): 11855-11870, 2011.
29. Kaufmann et al., “Structure of Penaeus stylirostris densovirus, a shrimp pathogen.” J Virol 84(21): 11289-11296, 2010.
30. Kaufmann et al., “Visualization of the externalized VP2 N termini of infectious human parvovirus B19.” J Virol 82(15): 7306-7312, 2008.
31. Kaufmann et al., “Structure of Bombyx mori densovirus 1, a silkworm pathogen.” J Virol 85(10): 4691-4697, 2011.
32. Ksiazek et al., “A novel coronavirus associated with severe acute respiratory syndrome.” N Engl J Med 348(20): 1953-1966, 2003.
33. Li et al., Genome organization of the densovirus from Bombyx mori (BmDNV-1) and enzyme activity of its capsid. J Gen Virol. 82(Pt 11): p. 2821-5, 2001.
34. Lightner et al., “Historic emergence, impact and current status of shrimp pathogens in the Americas.” J Invertebr Pathol 110(2): 174-183, 2012.
35. Liu et al., “The Acheta domesticus densovirus, isolated from the European house cricket, has evolved an expression strategy unique among parvoviruses.” J Virol 85(19): 10069-10078, 2011.
36. Mastronarde, “Advanced data acquisition from electron microscopes with SerialEM.” Microscopy and Microanalysis 24(S1): 864-865, 2018.
37. Meng et al., “The structure and host entry of an invertebrate parvovirus.” J Virol 87(23): 12523-12530, 2013.
38. Mietzsch et al., “Comparative Analysis of the Capsid Structures of AAVrh.10, AAVrh.39, and AAV8.” J Virol 94(6), 2020.
39. Mietzsch et al., Structural Insights into Human Bocaparvoviruses. J Virol. 91(11), 2017.40. Mietzsch et al., Structural Characterization of Cuta- and Tusavirus: Insight into Protoparvoviruses Capsid Morphology. Viruses. 12(6), 2020.
41. Mietzsch et al., “Twenty-Five Years of Structural Parvovirology.” Viruses 11(4), 2019.
42. Multeau et al., “Four amino acids of an insect densovirus capsid determine midgut tropism and virulence.” J Virol 86(10): 5937-5941, 2012.
43. Ng et al., “Structural characterization of the dual glycan binding adeno-associated virus serotype 6.” J Virol 84(24): 12945-12957, 2010.
44. Nigg and Falk, Diaphorina citri densovirus is a persistently infecting virus with a hybrid genome organization and unique transcription strategy. J Gen Virol. 101(2): p. 226-239, 2020.
45. Okonechnikov et al., “Unipro UGENE: a unified bioinformatics toolkit.” Bioinformatics 28(8): 1166-1167, 2012.
46. Pénzes et al., “Molecular biology and structure of a novel penaeid shrimp densovirus elucidate convergent parvoviral host capsid evolution.” Proc Natl Acad Sci USA 117(33): 20211-20222, 2020.
47. Penzes et al., Adeno-associated Virus 9 Structural Rearrangements Induced by Endosomal Trafficking pH and Glycan Attachment. J Virol. 95(19): p. e0084321, 2021.
48. Pénzes et al., “Reorganizing the family Parvoviridae: a revised taxonomy independent of the canonical approach based on host association.” Arch Virol 165(9): 2133-2146, 2020.
49. Pénzes and Kaelber, “Identification by cryoEM of a densovirus causing mass mortality in mass-reared larval darkling beetles (Zopobas morio)” bioRxiv (2022): 2022-05.
50. Pettersen et al., “UCSF Chimera—a visualization system for exploratory research and analysis.” J Comput Chem 25(13): 1605-1612, 2004.
51. Pham et al., “Comparative Genomic Analysis of Acheta domesticus Densovirus Isolates from Different Outbreaks in Europe, North America, and Japan.” Genome Announc 1(4), 2013.
52. Pigeyre et al., “Interaction of a Densovirus with Glycans of the Peritrophic Matrix Mediates Oral Infection of the Lepidopteran Pest Spodoptera frugiperda.” Viruses 11(9): 870, 2019.
53. Plevka et al., “Structure of a packaging-defective mutant of minute virus of mice indicates that the genome is packaged via a pore at a 5-fold axis.” J Virol 85(10): 4822-4827, 2011.
54. Richard et al., Mass mortality in freshwater mussels (Actinonaias pectorosa) in the Clinch River, USA, linked to a novel densovirus. Sci Rep. 10(1): p. 14498, 2020.
55. Roekring et al., Comparison of penaeid shrimp and insect parvoviruses suggests that viral transfers may occur between two distantly related arthropod groups. Virus Res. 87(1): p. 79-87, 2002.55.
56. Rossmann et al., Structural comparisons of some small spherical plant viruses. J Mol Biol. 165(4): p. 711-36, 1983.
57. Rumbos, and Athanassiou, “The Superworm, Zophobas morio (Coleoptera:Tenebrionidae): A ‘Sleeping Giant’ in Nutrient Sources.” J Insect Sci 21(2), 2021.
58. Shike et al., Infectious hypodermal and hematopoietic necrosis virus of shrimp is related to mosquito 37 brevidensoviruses. Virology. 277(1): p. 167-77, 2000.
59. Simpson et al., “The structure of an insect parvovirus (Galleria mellonella densovirus) at 3.7 A resolution.” Structure 6(11): 1355-1367, 1998.
60. Sivaram et al., Isolation and characterization of densonucleosis virus from Aedes aegypti mosquitoes and its distribution in India. Intervirology. 52(1): p. 1-7, 2009.
61. Subramanian et al., “Cryo-EM maps reveal five-fold channel structures and their modification by gatekeeper mutations in the parvovirus minute virus of mice (MVM) capsid.” Virology 510: 216-223, 2017.
62. Suchard et al., “Bayesian phylogenetic and phylodynamic data integration using BEAST 1.10.” Virus Evol 4(1): vey016, 2018.
63. Sukhumsirichart et al., Complete nucleotide sequence and genomic organization of hepatopancreatic parvovirus (HPV) of Penaeus monodon. Virology. 346(2): p. 266-77, 2006.
64. Summerford and Samulski, “Membrane-associated heparan sulfate proteoglycan is a receptor for adeno-associated virus type 2 virions.” J Virol 72(2): 1438-1445, 1998.
65. Tang et al., Geographic variations among infectious hypodermal and hematopoietic necrosis virus (IHHNV) isolates and characteristics of their infection. Dis Aquat Organ. 53(2): p. 91-9, 2003.
66. Tijssen, P., et al., Organization and expression strategy of the ambisense genome of densonucleosis virus of 25 Galleria mellonella. J Virol, 77(19): p. 10357-65. 2003.
67. Tokarev et al., Molecular Identification of a Densovirus in Healthy and Diseased Zophobas morio (Coleoptera, Tenebrionidae), in Intervirology. S. Karger AG, Basel.: Switzerland. p. 222-226, 2019.
68. Venkatakrishnan et al., “Structure and dynamics of adeno-associated virus serotype 1 VP1-unique N-terminal domain and its role in capsid trafficking.” J Virol 87(9): 4974-4984, 2013.
69. Walters et al., “Binding of adeno-associated virus type 5 to 2,3-linked sialic acid is required for gene transfer.” J Biol Chem 276(23): 20610-20616, 2001.
70. Yang et al., Characterization of the promoter elements and transcription profile of Periplaneta fuliginos densovirus nonstructural genes. Virus Res. 133(2): p. 149-56, 2008.
71. Yu and Tijssen, Gene expression of five different iteradensoviruses: Bombyx mori densovirus, Casphalia extranea densovirus, Papilio polyxenes densovirus, Sibine fusca densovirus, and Danaus plexippus densovirus. J Virol. 88(20): p. 12152-7, 2014.
72. Zadori et al., “A viral phospholipase A2 is required for parvovirus infectivity.” Dev Cell 1(2): 291-302, 2001.
73. Zhang et al., “Divergent engagements between adeno-associated viruses with their cellular receptor AAVR.” Nat Commun 10(1): 3760, 2019.
74. Zheng et al., “MotionCor2: anisotropic correction of beam-induced motion for improved cryo-electron microscopy.” Nat Methods 14(4): 331-332, 2017.

1. A method of inhibiting Zophobas morio black wasting disease morbidity and mortality in a darkling beetle colony in need thereof, comprising:
(a) isolating a strain of densovirus from Tenebrio molitor; and
(b) administering the strain of densovirus to the darkling beetle colony,
wherein the strain of densovirus is non-pathogenic to the recipient beetle colony.

2. The method of claim 1, wherein the strain of densovirus is SEQ ID NO:5.

3. The method of claim 1, wherein the administering is by injection, dripping, spraying or ingestion.

4. A prophylactic vaccine composition, comprising the corpses of Z. morio larvae infected with a non-pathogenic strain of densovirus selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and any combination thereof.

5. A prophylactic vaccine composition, comprising the corpses of darkling beetle larvae infected with a strain of ZmBWV that is isolated from Tenebrio molitor and does not cause mortality in the recipient species at a dose of 10⁹ genomes.

6. A prophylactic vaccine composition of claim 4, wherein the strain is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and any combination thereof.

7. A prophylactic vaccine composition of claim 5, wherein the darkling beetle is of the species Zophobas morio.

8. A prophylactic vaccine composition comprising purified virions of a non-pathogenic strain of densovirus and a pharmaceutically acceptable medium, wherein the sequence identity between the non-pathogenic strain of densovirus and strain NJ2-molitor (SEQ ID NO: 5) is about 96% or greater.

9. A prophylactic vaccine composition comprising: a pharmaceutically acceptable medium and a purified virions of a non-pathogenic strain of densovirus selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and any combination thereof.

10. A prophylactic vaccine composition, comprising one or more nonpathogenic densovirus strain, wherein the NS3 canonical ATG start codon is mutated, truncating the NS3 protein to fewer than 200 amino acids, instead of the length of 221 residues in the pathogenic, highly virulent strain UT-morio (SEQ ID NO: 1).

11. A prophylactic vaccine composition of claim 10, wherein the DNA sequence identity from NJ2-molitor (SEQ ID NO: 5) is about 96% or greater.

12. A prophylactic vaccine composition of claim 10, wherein the pharmaceutically acceptable medium is phosphate-buffered saline.

13. A prophylactic vaccine composition of claim 10, wherein the composition is formulated for injection, spraying, or dripping.


SEQUENCE LISTING (12 sequences)

SEQ ID NO: 1 - DNA, 5449 bp, Zophobas morio black wasting virus
  misc_feature 1..5449: note = Zophobas morio black wasting virus, strain UT-morio
  source 1..5449: mol_type = genomic DNA
gacgaccagatctatttatacccaatggtgaccttggaaacctggttaccatgtaaattttctatttactggtataccatggtttccaaggtcaccattgggtataaatagatctggtcgtcgtcgcagtcgtcagtgataagccaggggcttatgggcaagcaccaagtaagggggataaagtgaagtgtataaatagtgttcaacatgtaagtagctcattctgttgttgtatttcgtgcagtgcagtaatactttggtttggtaagtaggtttaaacataagaagatatatcaccctcagagcttgtatgacatgactaagaagaaggtaagagaggtgtacaagaataattggaacttgctggtactaccgaatcgtatccagagggaactactactagattggttaagatgtgacgagacaattccggagagtgatgacgatgtggaacgaatagtggcacgtatggaaaggggatgggaggcattgaagccctttggttcgactacatttgtgtacttgatgagattaccagatgaagtaccgccatttgcacatgaaaggaatcatatcatttgggacttttacttgtggtatgaacaagggcgagaaaagaaaatttgtgaaccgtgttattccgggaagagtcgtttctactgtccaggatctgctaatgaatggttagaaaaaggatgggtatttaaacgcgtagagaatcactccatgattgatggggacagattgttacaggagttgatatgggatgaagacaactggtgcagtttatgtcttgttgagccattatggatccatatattggatgatgatgattgtttgtttgattatgactatcatttgaagagaaggcgtacatggagtgactcaagctctgaggacagtgatattgattattgtaaacatactgttatgcaagggattcgtatgaaccccactttgtataaatttattaatgaataaagcaagtgttatagaattgttgtttcattacagatgaataatgacgaccacttttgggaatattttgacagtactctgggagatgcttccggacgagtgggcgaatcatccgggagtatggtggaagattatggatacgataccactggaccaacaatgcaagaatgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgacgaggacttatccttatatctatccacagcgatcaggatggcagtgcccatgtccacaccatccacagctgcgcctacgcaaacagggcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacaatgaacgaaaacgaggaattgaaccgaaaggtgatggaggtactcgaagtcgcaaaagaagaggtgttggcaacgcaggaggagatggaggaatcagaggggacacaggagtaattcttgatttaattaagaggtatgctgtttgtccattatctgagatagtatatactcgcgagtacttggaaaatcgtattgcagttaaaaggttagatgatagggatgtaaagaacgctatagattgccatgctgctattattaatacttggaatagagaggattatgttaagttctatgaggacccaaacactgttaaaatttggagtgcacgtagtattgacttggtggaattatattatttaactgataatgaatcgagggatattattaataaactgctggattaccaatgtgggttagctaagaaacagtttgtaacagatttgattaatgtaacagacatgaatattcccaagtgtaattgctttctaattgttagccctccaagtgctggcaagaactttctatgcgatgctattaaagattactatcttaatgttggacaaatgcagaaccctaataaatataataccttcgcgtatcaggattgtcacaatagacgacttcttatttggaatgaaccaaactacgaacctagagaaacggagaatttaaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctccgttcttgaaagaggtaataaaaaaaccacgacctgattctgtaatgaatttattatatgaaatatacaattcataattatggttttacagttttaatcatttgcatcctgccatatacattaggattatcatatactttagcaataggacgaccatcatttgcaaaatattgaagttgagatttggtattggccgagtaacatccgcccttaatgtatgtgtaagggtcgacactctctgttgttagtacacaatcaacttcgaagtagccttgtgcgtctagccaggaattagcttgagtagtttcatcaatggtagttaacttgggaacagctcttatgccaacatttatagatggcatttgagtgtcatgtactgtctcaacatttacctcctcgaatatacctccttgttcaatagggactcgaagatagttctgttcctcatcaaagaatgttgtatctgcattgggaccctgtatatatttgcgaggtgcattacgtacttgtgttggtggtgtagcttgtgaggaatcggttgttttcgctgccgtcacttcattctttgttcccgcaggataactagcattatagttttgagtgatcaagttgtttggtactggtgcaaactgtggtaccagaggtgcgtagctaaaatcgtaatccatggcaactacatctgtgttgatacatgcacttgcatcgaattcttctataaagtttttgtatggtggaaaccccgccaatatcttagtaccagtagtagcccttgcatcattagtgtaaatggttaaatagtcctgtagatatatctctgcgccagttgcatctgctggtggtttcttagcaaagtctggactgtcattatcgtaaccatacatagcaatcttgagaccatctcgatattcgtaggatgtcaatgttgcgaatcccgtaggttgcatgggttcagtatcagaatatgtatactttctgtttgtagagcatataaaaggaatacttctaatacccttagcaacttgcagaaatttgttttgattcagtgtagcattagccgtttgagtatctccagtttgaaatgcaactctagtattccatgttctaatacgtactgaagccgacttagcgaacgtcccaggatagttcttcattcgattatattctgctggactcatataaaagaataagtattcccatggaattgatgccattcctgtggttaatgcccatcttgctggtctttctccgctagctgcctcggccaagatgacgttggcatttgctgaagttaggaatctccatttctttctataagtttgttgaaacttttcaacatgaagtccaattggacgtaatatagctgtagctccgccttccccactacctcctctagccataccatccaaattgcctcctgttcctggtaaacttgtcccagacttggataccttactcggcggtccgctaccatcactacctcgcttggatccactactagagctagatacttgagccatttctacgtccgccatgtttggttgtggtgcgttacttggtccggcgtctcttaaatcttgtctatctcttacttgttgtaaaaaatcgtctactccgtcttctgattgctgactgggttgttccgcctcctggggtgaccctaaaagatccaaatcaaagttatcaattgcatcagcctgagttaaaggtaggtcgtcatccccaggaggatcaaaatattctccacgtcttactctagctaggttccattgttccatagcatagcgtctttgtccatcattcatactatcccaattaggacgatcttggggattcggacccagcggtctaactctaggcattgcttaccgtaatatacttttccacttagacgttcgactgcgtgcttaactcccaacccgactcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtactggatttccaggtccgatattgttcgaaaacggtaaaactagtccgttccgtggtggttctccccttgctagattggcagaatctcttctcgcctgcgcttccctttgttcttgcttcttcgccttgtttagtcccctgcgaggatttactagtctccagtgttctggatatttagccttagtgtccttattccaaggatctccgtacggtttgatcttctcctgtcgctctttcaaccccaaacgtctcaattcccaaggaacggctgcgtagtaatcgtctcgattaccaaatattgctctcgataaagggttgttagggtcagctgctgctcttcccaattgcccttcgaatattggttgatattggctacctgctccaataaccgcacccccaacacgtggtgtattaggtaccttcaatttactgtcacttccggcactggagatactactattagacgagttaataggaatagatacgtatccacccctacgtcgcacccctgtacttgtattgggttgattaaacacactctcttcactactgctgggggtaatagtgctcagctcgatgtactcggacatactgactggaaataacctgcgcatacatatttatatccatgttgatcccccttacttggtgcttgcccataagcccctggcttatcactgacgactgcgacgacgaccagatctatttatacccaatggtgaccttggaaaccggttaccatgtaaattttctatttacggtataccatggtttccaaggtcaccattgggtataaatagatctggtcgtcgtc

SEQ ID NO: 2 - DNA, 4920 bp, Zophobas morio black wasting virus
  misc_feature 1..4920: note = Zophobas morio black wasting virus, strain FL-morio
  source 1..4920: mol_type = genomic DNA
aaccggttaccatgtaaattttctatttacggtataccatggtttcaaggtcaccatttgggtataaatagatctggtcgtcgtcgcagtcgtcagtgataagccaggggcttatgggcaagcaccaagtaagggagataaagtgaagtgtataaatagtgttgaacatgtaagtagctcattctgttgttgtattttatacagtgcagtaatactttgctttggtaagtaggtttagacataagaagatatatcaccctcagagcttgtatgacattactaagaagaaggtaagagaggtgtacaagaataattggaacgagacgaaggtactagcgaagagtatccagagggaactactactagattggttaagatgtgacgagacaattcctgagagtgatgacgatgtggaacgaatagtggcacgtatggaaaggggatgggaggcattgaagccctttggtccgactacatttgtgtacttgatgagattaccagatgaagtaccgccatttgcacatgaaaggaatcatatcatttgggacttctatttgtggtatgaacaagggcgagaaaagaaaatttgtgaaccgtgttattccgggaagagtcgtttctaccgtccaggatctgctaatgaatggttagaaaaaggatgggtatttaaacgcgtagagaatcactccatgattgatggggatagattgttagaggatttgatatgggatgaagacaactggtgcagtttatgtattgttgagccattatggatccatatatttgatgatgatgattgtttgtttgattatgactatcatttgaagagaaggcgtacatggagtgactcaagctctgaggacagtgatattgattattgtaaacatactgttatgcaagggattcgtataaaccccactttgtataaatttattaatgaataaagcaagtgttatagaattgttgtttcattgcagatgaataatgacgaccacttttgggaatattttgacagtactctgggagatgcttccggacgagtgggcgaatcatccgggagtatggtggaagattatggatacgataccactggaccaacaatgcaagagtgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgacgaggacttatccttatatctatccacagcgatcaagatggcagtgcccatgtccacaccatccacagctgcgcctacgcaaacagggcgtgcaggtgctatttcaaagcgttccccgaagcgcaagaggacgctagacgacttcttcgaaagcctccatccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttaaaggaggatcacactcaggcctggagaactgccacgacccgcttgaccatcacgatgaacgaaaacgaggaattgaaccgaaaggtgatggaagtactcgaagccgcaaaagaagaggtgttggcaacgcaggaggagatggaggaatcagaggggacacaggagtaattcttgacttaattaagaggtatgctgtttgtccattatctgagatagtatatactcgcgagtacttggaaaatcgcattgcagttaaaaggttagatgatagggatgtgaagaacgctatagattgccatgctgctattattaatacttggaatagagaggattatgttaagttctatgaggacccaaacactgttaaaatttggagtgcacgtagtattgacttggtggagttatattatttaactgataatgaatctagagacattattaataaactgcttgattaccaatgtgggttagctaagaaacagtttgtaacggatttgattaatgtaacagacatgaatattcccaagtgtaactgctttctaattgttagccctccaagtgctggcaagaactttctatgcgatgctattaaagattactatcttaatgttggacaaatgcagaaccctaataaatataataccttcgcgtatcaggattgtcacaatagacgacttcttatttggaatgaaccaaactacgaacctagggaaacggagaatttgaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctccgtttttgaaagaggtaataaaaaaccacgacctgattctgtaatgaatttattatatgaaatatacaactcataattatggttttacagttttaatcatttgcatcctgccatatacattaggattatcatatactttagcaataggacgaccatcacttgcaaaatattgaagttgagatttggtattggccgagtaacatccgcccttaatgtatgtgtaagggtcgacactctctgttcttagtacacaatcaacttcgaagtagccttgtgcgtctagccaggaattagcttgagtagtttcatcaatggtagttaacttgggaacagctcttatgccaacatttatagatggcatttgagtgtcatgtactgtctcaacatttacctcctcgaatatacctccttgttcaatagggactcgaagatagttctgttcctcatcaaagaatgttgtatctgcatggggaccctgtatatatttgcgaggtgcattacgcacttgtgttggtactgtagcttgtgaggaatcggttgttttcgctgccgtcacttcattctttgttcccgcaggataactaccattatagttttgagtaatcaagttgtttggtactggtgcaaactgtggtaccagaggtgcgtagctaaaatcgtaatccatagcaactacatctgtgtttatacatgcacttgcatcgaattcttctataaagtttttgtatggtggaaaccccgccaatatcttagtaccagtagtagcccttgcgtcattagtgtaaatggttaaatagtcctgtagatatatctctgcgccagttgcatctgctggtggtttcttagcaaagtctggactgtcattatcgtaaccgtacatagcaatcttgagaccatctcgatattggtaggatgtcaatgttgcgaatccagtaggttgcataggttcagtatcagaatatgtatactttctgtttgtagagcatataaaaggaatacttctaatccccttagccacttgtaagaacttattctgatttagtgtagcattagctgtttgagtatctccagtttggaatgcaactcttgtgttccacgtcctaatacgtactgaggcggacttagcaaacgtaccaggatagttcttcattctgttatattctgctggactcatataaaagaataagtattcccatggaattgatgccattcctgtcgttagtgcccatcttgctggtctgtctccgctagccgcttcagccaatatgacgtttgcatttgctgaagtcagaaacctccatttctttctataagtctgttgaaacttctcaacatgaagtccaattggactaatatagctgtagctccgccttccccactacctcctctaaccatgccatctaagttgcctccagttccagggagactcgtccctgacttggataccttactcggcggtccgctaccatcactgcctcgcttcgatccaccgctggaacttgatacttgagacatttctacgtccgccatgtttggttgtggtgcgttacttggtccggcgtctcttaaatcttgtctatctcttacttgttgtaaaaaatcgtctactccgtcttctgattgctgactgggttgttccgcctcctggggtgaccctaaaagatccaaatcaaaattatctattgcatcagcctgagttaatggttggtcgtcatccccaggaggattaaagtattccccacgtcttactctagctaggttccattgctccatagcataacgtctttgtccctcgttcatactatcccaattaggacgatcctgaggattcggacccaacggtctaactctaggcattgcttaccgtagtatacttttccgcttaaatgttcgactgcgtgcttaactcccagcccgactcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttgctcgcgggacgtatgggatttccaggtccgatattgttcgaaaatggtaaaactagtccgttccgtggtggttctccccttgctagattggcagaatctcttctcgcctgtgcttccctttgttcttgcttcttcgccttgtttagtcccctgcgaggatttactagcctccagtgttctggatatttggctttagtgtccttattccaagggtctccgtagggtttgatcttctcttgtcgatctttcaaccccaatcgtctcaattcccaaggaacggctgcgtagtaatcgtctcgattgccaaaaattgctcttgacaaggggttattaggg

SEQ ID NO: 3 - DNA, 5463 bp, Zophobas morio black wasting virus
  misc_feature 1..5463: note = Zophobas morio black wasting virus, strain LA-molitor
  source 1..5463: mol_type = genomic DNA
gacgacgaccagatctatttatacccaatggtgaccttggaaaccatggtataccatgtaaatagtaaatttacatggtataccatggtttccaaggtcaccattgggtataaatagatctggtcgtcgtcgcagtcgtcagtgataagccaggggcttatgggcaagcaccaagtaagggagataaagtgaagtgtataaatagtgttcaacatgtaagtagctcattctgttgttgtatttcgtgcagtccagtaacactttggtttggtaagtaggtttaaacataagaagatatatcaccctcagagcttgtatgacattactaagaagaaggtaagagaggtgtacaagaataattggaacgagacgaaggtactaccgaggactatccagagggaactattactagattggttaagatgtgacgagaaaatttctgagagtgatgacgatgtggaacgaatagtggcacgtatggaaaggggatgggaggcattgaagccctttggtccgactacatttgtgtacttgatgagattaccagatgaagtaccgccatttgcacatgaaaggaatcatatcatttgggacttctatttgtggtatgaacaagggcgagaaaagaaaatttgtgaaccgtgttattccgggaagagtcgtttctaccgtccaggatctgctaatgaatggttagaaaaaggatgggtatttaaacgcgtagagaatcactccatgattgatggggacagattgttagaggatttgatatgggatgaagacaactggtgcagtttatgcattgttgagccattatggatccatatatttgatgatgatgattgtttgtttgattatgactatcatttgaagagaaggcgtacatggagtgactcaagctctgaggacagtgatattgattattgtaaacatactgttatgcaagggattcgtataaaccccactttgtataaatttattaatgaataaagcaagtgttatagaattgttgtttcattacagatgaataatgacgaccacttttgggaatattttgacagtactctgggagatgcttccggacgagtgggcgaatcatccgggagtatggtggaagattatggatacgataccactggaccaacaatgcaagaatgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgaagaggacttatcctcatatctattcacagcgatcaagatggcggtgcccatatccacaccatccacagctgcgcctacgcaaacagggcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacgatgaacgaaaacgaggaattgaaccgaaaggtgatggaggtactcgaagtcgcaaaagaagaggtgttggcaacgcaggaggagatggaggaatcagaggggacacaggagtaattcttgatttaattaagaggtatgctgtttgtccattatctgagatagtatatactcgcgagtacttggaaaatccccttgcagttaaaaggttagatgatagggatgtaaagaacgctatagattgccatgctgctattattaatacttggaatagagaggattatgttaagttttatgaggacccaaaaactgttaaaatttggagtgcacgtaatattgactcggtggagttatattatttaactgataatgaatctagagatattattaataaactgcttgattatcaatgtgggttagctaagaaacagtttgtaacggacttgattaatgtaacagacatgaatattcccaagtgtaattgctttctaattgttagccctccaagtgctggcaagaactttctatgcgatgctattaaagattactatattaatgttggacaaatgcagaaccctaataaatataataccttcgcgtatcaggattgtcacaatagacgacttcttatttggaatgaaccaaactacgaacctagggaaacggagaatttgaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctcctttcttaaaagaggtaataaaaaaaccacgacctgattctgtaatgaatttattatatgaaatatacaattcataattatggtttcacagttttaatcatttgcatcctgccgtatacattaggattatcatatactttagcaataggacgaccatcacttgcaaaatattgaagttgagatttggtattggccgagtaacatccgcccttaatgtatgtgtaagggtcgacactctctgttgttaatacacaatcaacttcgaaatagccttgtgcgtctagccaggaattagcttgagtagtttcatcaatggtagttaacttgggaacagctcttatgccaacatttatagatggcatttgagtgtcatgtactgtctcaacatttacctcctcgaatataccaccttgttcaatagggactcgaagatagttctgttcctcatcaaagaacgttgtatctgcatggggaccctgtatatatttgcgaggtgcattacgtacttgtgttggtgctgtagcttgtgaggaatcggttgttttcgctgccgtcacttcattctttgttcccgcaggataactaccattatagttttgagtaatcaagttgtttggtactggtgcaaactgcggtaccagaggtgcatagctaaaatcgtaatccatagcaactacatctgtgtttatacatgcacttgcatcgaattcttctataaagtttttgtatggtggaaaccccgccaatatcttagtaccagtagtagcccttgcatcattagtgtaaatggttaaatagtcctgcagatatatctctgcgccagttgcatctgctggtggtttcttagcaaagtctgcactgtcattatcgtaaccatacatagcaatcttgagaccatctcgatattggtaggatgtcaatgttgcgaatcccgtaggttgcatgggttcagtatcagaatatgtatactttctgtttgtagaacatataaaaggaatacttctaatacccttagcaacttgcagaaatttgttttgattcagtgtagcattagccgtttgagtatctccagtttggaatgcaactcttgtgttccacgtcctaatacgtactgaagcggacttagcaaacgtaccaggatagtttttcattctgttatattctgctggactcatatagaagaataagtattcccatggaattgatgccattcctgtcgttagcgcccatcttgctggtctttctccgctagctgcctcggccaagatgacgttggcatttgctgaagttaggaatctccatttctttcggtatgtttgttgaaatttttcgacgtgtaatccaattggacgtaatatagctgtagctccgccttccccactacctcctctaaccataccatccaaattgcctcctgttcctggtaaacttgtcccagacttggataccttactcggcggtccgctaccatcactacctcgcttggatccactactagagctagatactcgagccatttctacgtccgccatgtttggttgtggtgcgttacttggtccggcgtctcttaaatcttgtctatctcttacttgttgtaaaaaatcgtctactccgtcttccgattgctgactgggttgttccgcctcctggggtgaccctaaaagatccaaatcaaagttatcaatcgcatcagcctgagttaaaggtaggtcgtcatccccaggaggatcaaagtattccccacgtcttactctagctaggttccattgttccatagcataacgtctttgtccctcattcatactatcccaattaggacgatcctgaggattcggacccaacggtctaactctaggcattgcttaccgtagtatacttttccgcttaaatgttcgactgcgtgcttaattcccagcccgattcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtattggatttccaggtccgatattgttcgaaaacggtaaaactagtccgttccgtggtggttctccccttgctagattggcagaatctcttctcgcctgtgcttccctttgttcttgcttcttcgccttctttagtcccctgcgaggatttactagtctccagtgttctggatatttagccttagtgtccttattccaaggatctccgtacggtttgatcttctcctgtcgctctttcaaccccaaacgtctcaattcccaaggaacggctgcgtagtaatcgtctcgattaccaaatattgctctcgataaagggttgttagggtcagctgctgctcttcccaattgcccttcgaatattggttgatattggctacctgctccaataaccgcacccccaacacgtggtgtattaggtactctcaatttactgtcactgccggcactggagatactactattagacgagttaataggaatagatacgtatccacccctacgtcgcacccctgtacttgtactgggttgattaaacacactctcttcactactgctgggggtaatagtgctcagctcgatgtactcggacatactgactggaaataacctgcgcatacatatttatatccatgttgatcccccttacttggtgcttgcccataagcccctggcttatcactgacgactgcgacgacgaccagatctatttatacccaatggtgaccttggaaaccatggtataccatgtaaatataaaatttacatggtataccatggtttccaaggtcaccattgggtataaatagatctggtcgtcgtc

SEQ ID NO: 4 - DNA, 5447 bp, Zophobas morio black wasting virus
  misc_feature 1..5447: note = Zophobas morio black wasting virus, strain NJ1-molitor
  source 1..5447: mol_type = genomic DNA
agatctatttatacccaatggtgaccttggaaaccatggtataccatgtaaatagtaaatttacatggtataccatggtttccaaggtcaccattgggtataaatagatctgctcgtcgtcgcagtcgtcagtgataagccaggggcttatgggcaagcaccaagtaagggagataaagtgaagtgtataaatagtgtttaacatgtaagtacctcattctgttgttgtatttcgtgcagtgcagtaatactttggtttggtaagtaggtttaaacataagaagatatatcaccctcagagcttgtatgacattactaagaagaaggtaagagaggtgtacaagaataattggaacgagacgaaggtgctaccgaagactatccagagggaactactactagattggttaagatgtgacgagaagatttctgagagtgatgacgatgtggaacgaatagtggcacgtatggaaaggggatgggaggcattgaagccctttggtccgaccgcatttgtgtacttgatgagattaccagatgaagtaccgccatttgcacatgaaaggaatcatatcatttgggacttttatttgtggtatgaacaagggcgagaaaagaaaatttgtgaaccgtgttattccgggaagagtcgtttctaccgtccaggatctgctaatgaatggttggaaaaaggatgggtatttaaacgcgtagagaatcactccatgattgatggggacagattgttagaggatttgatatgggatgaagacaactggtgcagtttgtgtattgttgagccattatggatccatatacttgatgatgatgattgtttgtttgattatgactatcatttgaagagaagacgtacatggagtgactcaagctctgaggacagtgatattgattattgtaaacatactgttatgcaagggattcgtataaaccccactttgtataaatttattaatgaataaagcaagtgttatagaattgttgtttcattacagatgaataatgacgaccacttttgggaatattttgacagtactctgggagatgcttccggacgagtgggcgaatcatccgggagtatggtggaagattatggatacgataccactggaccaacaatgcaagaatgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgacgaggacttatccttatatctatccacagcgatcaggatagcagtgcccatgtccacaccatccacagctgcgcctacgcaaacaggtcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacgatgaacgaaaacgaggaattgaatcgaaaggtgatggaggtactcgaagtcgcaaaagaagaggtgttggcaacgcaggaggagatggaggaatcagaggggacacaggagtaattcttgatttaattaagaggtatgctgtttgtccattatctgagatagtatatactcgcgagtacttggaaaatcccattgcagttaaaagattagatgatagggatgtaaagaacgctatagattgccatgctgctattattaatacttggaatagacaggattatgttaagttttatgaggacccaaacactgttaaaatttggagtgcacgtaatgttgactcggtagagttatattatttaactgataatgaatctagagatattattaataaactgcttgattatcaatgtgggttagctaagaaacagtttgtaacggatttgattaatgtaacagacatgaatattcccaagtgtaattgctttctaattgttagccctccaagtgctggcaagaactttctatgcgatgctattaaagattactatattaatgttggacaaatgcagaaccctaataaatataatactttcgcgtatcaggattgtcacaatagacgacttcttatttggaatgaaccaaactacgaacctagggaaacggagaatttgaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctcctttcttaaaagaggtaataaaaaagccacgtcctgattctgtaatgaatttattatatgaaatatacaattcataattatggttttacagttttaatcatttgcatcctgccatatacattaggattatcatatactttagcaataggacgaccatcacttgcaaaatattgaagttgagatttggtattggccgagtaacatccgcccttaatgtatgtgtaagggtcgacactctctgttgttagtacacaatcaacttcgaagtagccttgtgcgtctagccaggaattagcttgagtagtttcatcaatggtagttaacttgggaacagctcttatgccaacatttatagatggcatttgagtatcatgtactgtctcaacatttacctcctcgaatataccaccttgttcaatagggactcgaagatagttctgttcctcatcgaagaatgttgtatctgcgtggggaccctgtatatatttgcgaggtgcattacgtacttgtgttggtgctgtagcttgtgaggaatcggttgttttcgctgccgtcacttcattctttgttcccgcaggataactaccattataattttgagtaatcaagttgtttggtactggtgcaaactgcggtaccagaggtgcgtagctaaaatcgtaatccatagcaactacatctgtgttgatacatgcacttgcatcgaattcttctataaagtttttgtatggtggaaaccccgccaatatcttagtaccagtagtagcccttgcatcattagtgtaaatggttaaatagtcctgtagatatatctctgcgccagttgcatctgctggtggtttcttagcaaagtctttattatcgttatcgtaaccatacatagcagtcttgagaccatctcgatattggtaggatgtcaatgttgcgaatcccgtaggttgcatgggttcagtatcagaatatgtatactttctgtttgtagaacatataaaaggaatacttctaatacccttagcaacttgcagaaatttgttttgattcagtgtagcattagccgtttgagtatctccagtttgaaatgcaactctagtattccatgttctaatacgtactgaagccgacttagcaaacgtaccaggatagttcttcattcgattatattctgctggactcatataaaagaataagtattcccatggaattgatgccattcctgtcgttagcgcccatcttgctggtctttctccgctggccgcttcagccaatatgacgtttgcatttgctgaagtcagaaacctccatttctttctataagtttgttgaaacttctcaacatgaagtccaattggacgtaatatagctgtagctccgccttccccactacctcctctaaccatgccatccaaattgcctccagttccagggagactcgtccctgacttggataccttactcggcggtccgccaccaccactgcctcgcttcgatccaccgctagaacttgatacttgagacatttctatgtctgccatgtttggttgtggtgcgttacttggtccggcgtctcttaaatcttgtctatctcttacttgttgtaaaaaatcgtctactccgtcttctgattgctgactgggttgttccgcctcctggggtgaccctaaaagatccaaatcaaagttatcaatcgcatcagcctgagttaaaggtaggtcgtcatccccaggaggatcaaagtattccccacgtcttactctagctaggttccattgttccatagcataacgtctttgtccctcattcatactatcccaattaggacgatcctgaggattcggacccaacggtctaactctaggcattgcttaccgtagtatacttttccacttaaatgttcgactgcgtgcttaactcccagcccgattcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtattggatttccaggtccgatattgttcgaatacggtaaaactagtccgttccgtggtggttctccccttgctagattggcagaatctcttctcgcctgtgcttccctttgttcttgctttttcaccttctttagtcccctgcgaggatttactagtctccagtgttctggatatttagccttagtgtccttattccaaggatctccgtacggtttgatcttctcctgtcgctctttcaaccccaaacgtctcaattcccaaggaacggctgcgtagtaatcgtcccgattaccaaatatcgctctcgataaagggttcctagggtcagctgctgctcttcccaattgcccttcgaatattggttgatattggctacctgctccaataaccgcacccccaacacgtggtgtattaggtactctcaatttactgtcacttccggcactggagatactactattagacgagttaataggaatagatacgtatccacccctacgtcgcacccctgtacttgtactgggttgattaaacacactctcttcactactgctgggggtaatagtgctcagctcgatgtactcggacatactgactggaaataacctgcgcatacatatttatatccatgttgatcccccttacttggtgcttgcccataagcccctggcttatcactgacgactgcgacgacgagcagatctatttatacccaatggtgaccttggaaaccatggtataccgtaaatagaaaatttacatggtaaccggtttccaaggtcaccattgggtataaatagatctgctcgtcgt

SEQ ID NO: 5 - DNA, 5473 bp, Zophobas morio black wasting virus
  misc_feature 1..5473: note = Zophobas morio black wasting virus, strain NJ2-molitor
  source 1..5473: mol_type = genomic DNA
actgcgacgacgagcagatctatttatacccaatggtgaccttggaaaccatggtataccatgtaaattttctatttacggtataccatggtttccaaggtcaccattgggtataaatagatctgctcgtcgtcgcagtcgtcagtgataagccaggggcttatgggcaagcaccaagtaagggagataaagtgaagtgtataaatagtgtttaacatgtaagtacctcattctgttgttgtatttcgtgcagtgcagtaatactttggtttggtaagtaggtttaaacataagaagatatatcaccctcagagcttgtatgacattactaagaagaaagtaagagaggtgtacaagaataattggaacgagacgaaggtactaccgaagactatccagagggaactactactagattggttaagatgtgacgagagaattcctgagagtgatgacgatgtggaacgaatagtggcacgtatggaaaggggatgggaggcattgaagccctttggtccgactacatttgtctacttgatgagattaccagatgaagtaccgccatttgcacatgacaggaatcatatcatttgggacttctatttgtggtatgaacaagggcgagaaaagaaaatttgtgaagcgtgttattccgggaagagtcgtttctaccgtccaggatctgctaatgaatggttagaaagaggatgggtatttaaacgcgtagagaatcactccatgattgatggggacagattgttagaggatttgatatgggatgaagacaactggtgcagtttatgtattgttgagccattatggatccatatatttgatgatgatgattgtttgtttgattacgactatcatttgaagagaaggcgtacatggagtgactcaagctctgaggacagtgatattgattattgtaaacatactgttatgcaagggattcgtataaaccccactttgtataaatttattaatgaataaagcaagtgttatagaattgttgtttcattgcagatgaataatgacgaccacttttgggaatattttgacagtactctgggagatgcttccggacgagtgggcgaatcatccgggagtatggtggaagattatggatacgataccactggaccaacagtgcaagaatgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgacgaggacttatccttatatctatccacagcgatcaagatagcagtgcccatgtccacaccatccacagctgcgcctacgcaaacaggtcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacgatgaacgaaaacgaagaattgaaccgaaaggtgatggaggtactcgaagtcgcaaaagaagaggtgttggcaacgcaggaggagatggaggaatcagaggggacacaggagtaattcttgatttaattaagaggtatgctgtttgtccattatctgagatagtatatactcgcgagtacttggaaaatccccttgcagttaaaaggttagatgatagggatgtaaagaacgctatagattgccatgctgctattattaatacttggaatagagaggattatgttaagttttatgaggacccaaacactgttaaaatttggagtgcacgtaatattgactcggtggagttatattatttaactgataatgaatctagagatattattaataaactgcttgattatcaatgtgggttagctaagaaacagtttgtaacggatttgattaatgtaacagacatgaatattcccaagtgtaattgctttctaattgttagccctccaagtgctggcaagaactttctatgcgatgctattaaagattactatattaatgttggacaaatgcagaaccctaataaatataataccttcgcgtatcaggattgtcacaatagacgacttcttatttggaatgaaccaaactacgaacctagggaaacggagaatttgaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctccgttcttgaaagaggtaataaaaaaaccacgacctgattctgtaatgaatttattatatgaaatatacaattcataattatggttttacagttttaatcatttgcatcctgccatatacattaggattatcatatactttagcaataggacgaccatcacttgcaaaatattgaagttgagatttggtattggccgagtaacatccgcccttaatgtatgtgtaagggtcgacactctctgttgttagtacacaatcaacttcgaagtagccttgtgcgtctagccaggaatttgcttgagtagtttcatcaatggtagttaacttgggaacagctcttatgccaacatttatagatggcatttgagtgtcatgtactgtctcaacgtttacctcctcgaatataccaccttgttcaatagggactcgaagatagttctgttcctcatcgaagaatgtcgtatctgcgtggggaccctgtatatacttgcgaggtgcattacgtacttgtgttggtgctgtagcttgtgaggaatcggttgttttcgctgccgtcacttcattctttgttcccgcaggataactaccattatagttttgagtaatcaagttgtttggtactggtgcaaactgtggtaccagaggtgcgtagctaaaatcgtaatccatagcaactacatctgtgttgatacatgcacttgcatcgaattcttctataaagtttttgtatggtggaaaccccgccaatatcttagtaccagtggtagcccttgcatcattagtgtaaatggttaaatagtcctgtagatatatctctgcgccagttgcatctgctggtggtttcttagcaaagtctgcactgtcattatcgtaaccatacatagcaatcttgagaccatctcgatattggtaggatgtcaatgttgcgaatcccgtaggttgcatgggttcagtatcagaatatgtatactttctgtttgtagaacatataaaaggaatacttctaatacccttagcaacttgcagaaatttgttttgattcagtgtagcattagccgtttgagtatctccagtttgaaatgcaactctagtattccatgttctaatacgtactgaagccgacttagcaaacgtcccaggatagttcttcattctgttatattctgctggactcatatagaagaataagtattcccatggaattgatgccattcctgtagttaatgcccatcttgctggtctgtctccgctggccgcttcagccaatatgacgtttgcatttgctgaagtcagaaacctccatttctttctataagtttgttgaaacttctcaacatgaagtccaattggacgtaatatagctgtagctccgccttccccactacctcctctaaccataccatctaagttgcctccagttccagggagactcgtccctgacttggataccttactcggcggtccgccaccaccactacctcgcttcgatccaccgctagaacttgatacttgagacatttctatgtccgccatgtttggttgtggtgcgttacttggtccggcgtctcttaagtcttgtctatctcttacttgttgtagaaaatcgtctactccgtcttctgattgctgactgggttgttccgcctcctggggtgaccctaaaagatccaaatcaaagttgtcaatcgcatcagcctgagttaaaggtaggtcgtcatccccaggaggatcaaagtattccccacgtcttactctagctaggttccattgttccatagcataacgtctttgtccctcattcatactatcccaattaggacgatcctgaggattcggacccagcggtctaactctaggcattgcttaccgtagtatacttttccacttaaacgttcgactgcgtgcttaactcccagcccgattcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtattggatttccaggtccgatattgttcgaaaacggtaaaactagtccgttccgtggtggttctccccttgctagattggcagaatctcttctcgcctgtgcttccctttgttcttgcttcttcaccttctttagtcccctgcgaggatttactagtctccagtgttctggatatttagccttagtgtccttattccaaggatctccgtacggtttgatcttctcctgtcgctctttcaaccccaaacgtctcaattcccaaggaacggctctgtagtaatcgtctcgattaccaaatattgctctcgataaagggttgttagggtcagctgctgctcttcccaattgcccttcgaatattggttgatattggctacctgctccaataaccgcacccccaacacgtggtgtattaggtactctcaatttactgtcacttccggcactggagatactactattagatgagttaataggaatagatacgtatccacccctacgtcgcacccctgtacttgtactgggttgattaaacacactctcttcactactgctgggggtaatagtgctcagctcgatgtactcggacatactgactggaaataacctgcgcatacatatttatatccatgttgatcccccttacttggtgcttgcccataagcccctggcttatcactgacgactgcgacgacgagcagatctatttatacccaatggtgaccttggaaaccatggtataccatgtaaattgaaaatttacatggtaacctggtttccaaggtcaccattgggtataaatagatctgctcgtcgtcgcagtcgtc

SEQ ID NO: 6 - DNA, 5243 bp, Zophobas morio black wasting virus
  misc_feature 1..5243: note = Zophobas morio black wasting virus, strain OH-molitor
  source 1..5243: mol_type = genomic DNA
gtcgtcagtgataagccaggggcttatgggcaagcaccaagtaagggagataaagtgaagtgtataaatagttttgaacatgtaagtagctcattctgttgttgtatttcgtgcagtgcagtaatactctgctttggtaagtaggtttaaacataagaagatatatcaccctcagagcttgtatgacattactaagaagaaggtaagagaggtgtacaagaataattggaacgagacgaaggtgctaccgaagactatccagagggaactattactagattggttaagatgtgacgagaagatttctgagagtgatgacgatgtggaacgaatagtggcacgtatggaaaggggatgggaggcattgaagccctttggtccgaccacatttgtgtacttgatgagattaccagatgaagtaccgccatttgcacatgaaaggaatcatatcatttgggacttctacttgtggtatgaacaagggcgagaaaagaaaatttgtgaaccgtgttattccgggaagagtcgtttctaccgtccaggatctgctaatgaatggttggaaaaaggatgggtatttaaacgcgtagagaatcactccatgattgatggggacagattgttagaggatttgatatgggatgaagacaactggtgcagtttatgtattgttgagccattatggatccatatacttgatgatgatgattgtttgtttgattatgactatcatttgaagagaaggcgtacatggagtgactcaagctctgaggacagtgatattgattattgtaaacatactgttatgcaagggattcgtataaaccccactttgtataaatttattaatgaataaagcaagtgttatagaattgttgtttcattacagatgaataatgacgaccacttttgggaatattttgacagtactctgggagatgcttccggacgagtgggcgaatcatccgggagtatggtggaagattatggatacgataccactggaccaacagtgcaagaatgcgatgttgcagctaatagggcgttggagcaagaattacaaacaatggtcgaccggttcgttacccgccttgaaaaagaagattggcaagacagcggatactatgtgtcagatgtctttgcctgcgaatcaattggacgagctcaaggattggctaagcgaatggctgaacgagcgggaaatttccgacgaggacttatccttatatctatccacagcgatcaagatagcagtgcccatgtccacaccatccacagctgcgcctacgcaaacaggtcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacgatgaacgaaaacgaggaattgaaccgaaaggtgatggaggtactcgaagtcgcaaaagaagaggtgttggcaacgcaggaggagatggaggaatcagaggggacacaggagtaattcttgatttgattaagaggtatgctgtttgtccattatctgagatagtatatactcgcgagtacttggaaaatcccattgcagttaaaaggttagatgatagggatgtaaagaacgctatagattgccatgctgctattattaatacttggaatagacaggattatgttaagttttatgaggacccaaacactgttaaaatttggagtgcacgtaatgttgactcggtggagttatattatttaactgataatgaatctagagatattattaataaactgcttgattatcaatgtgggttagctaagaaacagtttgtaacggatttgattaatgtaacagacatgaatattcccaagtgtaattgctttctaattgttagccctccaagtgctggcaagaactttctatgcgatgctattaaagattactatattaatgttggacaaatgcagaaccctaataaatataataccttcgcgtatcaggattgtcacaatagacgacttcttatttggaatgaaccaaactacgaacctagggaaacggagaatttgaagatgttatttggaggggataacttgtctgctaatgttaagtgtaaacctcaagcgaatgtaaagcgtactccagtaatatgtatgagtaatgttgtacccagatttgctaatcatgaagcatttgctgacagagttattacatattattggaatgctgctcctttcttaaaagaggtaataaaaaagccacgacctgattctgtaatgaatttattatatgaaatatacaattcataattatggttttacagttttaatcatttgcatcctgccatatacattaggattatcatatactttagcaataggacgaccatcacttgcaaaatattgaagttgagatttggtattggccgagtaacatccgcccttaatgtatgtgtaagggtcgacactctctgttgttagtacacaatcaacttcgaagtagccttgtgcgtctagccaggaattagcttgagtagtttcatcaatggtagttaacttgggaacagctcttatgccaatatttatagatggcatttgagtgtcatgtactgtctcaacatttacctcctcgaatatacctccttgttcaatagggactcgaagatagttctgttcctcatcgaagaatgttgtatctgcattgggaccctgtatatatttgcgaggtgcattacgtacttgtgttggtgctgtagcttgtgaggaatcggttgttttcgctgccgtcacttcattctttgttcccgcaggataactaccattatagttttgagtaatcaagttgttcggtactggtgcaaactgtggtaccagaggtgcgtagctaaaatcgtaatccatagcaactacatctgtgttgatacatgcacttgcatcgaattcttctataaagtttttgtatggtggaaaccccgccaatatcttagtaccagtagtagcccttgcatcattagtgtaaatggttaaatagtcctgtagatatatctctgcgccagttgcatctgctggtggtttcttagcaaagtctttattgtcgttatcgtaaccatacatagcagtcttgagaccatctcgatattggtatgatgtcaaggttgcgaatccagtaggttgcatgggttcagtatcagaatatgtatactttctgtttgtagagcatataaaaggaatacttctaatccccttagccacttgtaagaatttattctgatttagtgtagcattagccgtttgagtatctccagtttggaatgcaactcttgtgttccacgtcctaatacgtactgaagcggacttagcaaacgtcccaggatagttcttcattcgattatattctgctggactcatataaaagaataagtattcccatggaattgatgccattcctgtagttaatgcccatcttgctggtctttctccgctagctgcctcggccaatatgacgtttgcatttgctgaagtcagaaacctccatttctttctataagtttgttgaaacttctcaacatgaagtccaattggacgtaatatagctgtagctccgccttccccactacctcctctaaccataccatctaagttgcctccagttccagggagactcgtccctgacttggataccttactcggcggtccgccaccaccactgcctcgcttcgatccaccgctagaacttgatacttgagacatttctatgtccgccatgtttggttgtggtgcgttacttggtccggcgtctcttaaatcttgtctatctcttacttgttgtaaaaaatcgtctactccgtcttctgattgctgactgggttgttccgcctcctggggtgaccctaaaagatccaaatcaaagttatcaatcgcatcagcctgagttaaaggtaggtcgtcatccccaggaggatcaaagtattccccacgtcttactctagctaggttccattgttccatagcataacgtctttgtccctcattcatactatcccaattaggacgatcctgaggattcggacccaacggtctaactctaggcattgcttaccgtagtatacttttccgcttaaatgttcgactgcgtgcttaattcccagcccgattcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtattggatttccaggtccgatattgttcgaatacggtaaaacaagtccgttccgtggtggttctccccttgctagattggcagaatctcttctcgcctgtgcttccctttgttcttgcttctttaccttctttagtcccctgcgaggatttactagtctccagtgttctggatatttagccttagtgtccttattccaaggatctccgtacggtttgatcttctcctgtcgctctttcaaccccaaacgtctcaattcccaaggaacggctctgtagtaatcgtctcgattaccaaatattgctctcgataaagggttgttagggtcagctgctgctcttcccaattgcccttcgaatattggttgatattggctacctgctccaaaagccgcacccccaacacgtggtgtattaggtactctcagtttactgtcacttccggcactggagatactactattagacgagttaataggaatagatacgtatccacccctacgtcgcacccctgtacttgtactgggttgattaaacacactctcttcactactgctgggggtaatagtgctcagctcgatgtactcggacatactgactggaaataacctgcgcatacatatttatatccatgttgatcccccttacttggtgcttgcccataagcccctggcttatcactgacgactgcgacgacgagcagatctatttatacccaatggtgaccttggaaacc

SEQ ID NO: 7 - DNA, 5475 bp, Zophobas morio black wasting virus
  misc_feature 1..5475: note = Zophobas morio black wasting virus, strain OR molitor
  source 1..5475: mol_type = genomic DNA
actgcgacgacgagcagatctatttatacccaatggtgaccttggaaaccagggtataccagtaaattgtatatttacatggtataccatggtttccaaggtcaccattgggtataaatagatctgctcgtcgtcgcagtcgtcagtgataagccaggggcttatgggcaagcaccaagtaagggagataaagtgaagtgtataaatagtgtttaacatgtaagtacctcattctgttgttgtatttcgtgcagtgcagtaatactttggtttggtaagtaggtttaaacataagaagatatatcaccctcagagcttgtatgacattactaagaagaaggtaagagaggtgtacaagaataattggaacgagacgaaggtgctaccgaagactatccagagggaactactactagattggttaagatgtgacgagaagatttctgagagtgatgacgatgtggaacgaatagtggcacgtatggaaaggggatgggaggcattgaagccctttggtccgaccgcatttgtgtacttgatgagattaccagatgaagtaccgccatttgcacatgaaaggaatcatatcatttgggacttttatttgtggtatgaacaagggcgagaaaagaaaatttgtgaaccgtgttattccgggaagagtcgtttctaccgtccaggatctgctaatgaatggttggaaaaaggatgggtatttaaacgcgtagagaatcactccatgattgatggggacagattgttagaggatttgatatgggatgaagacaactggtgcagtttgtgtattgttgagccattatggatccatatacttgatgatgatgattgtttgtttgattatgactatcatttgaagagaagacgtacatggagtgactcaagctctgaggacagtgatattgattattgtaaacatactgttatgcaagggattcgtataaaccccactttgtataaatttattaatgaataaagcaagtgttatagaattgttgtttcattacagatgaataatgacgaccacttttgggaatattttgacagtactctgggagatgcttccggacgagtgggcgaatcatccgggagtatggtggaagattatggatacgataccactggaccaacaatgcaagaatgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgacgaggacttatccttatatctatccacagcgatcaggatagcagtgcccatgtccacaccatccacagctgcgcctacgcaaacaggtcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacgatgaacgaaaacgaggaattgaaccgaaaggtgatggaggtactcgaagtcgcaaaagaagaggtgttggcaacgcaggaggagatggaggaatcagaggggacacaggagtaattcttgatttaattaagaggtatgctgtttgtccattatctgagatagtatatactcgcgagtacttggaaaatcccattgcagttaaaagattagatgatagggatgtaaagaacgctatagattgccatgctgctattattaatacttggaatagacaggattatgttaagttttatgaggacccaaacactgttaaaatttggagtgcacgtaatgttgactcggtagagttatattatttaactgataatgaatctagagatattattaataaactgcttgattatcaatgtgggttagctaagaaacagtttgtaacggatttgattaatgtaacagacatgaatattcccaagtgtaattgctttctaattgttagccctccaagtgctggcaagaactttctatgcgatgctattaaagattactatattaatgttggacaaatgcagaaccctaataaatataatactttcgcgtatcaggattgtcacaatagacgacttcttatttggaatgaaccaaactacgaacctagggaaacggagaatttgaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctcctttcttaaaagaggtaataaaaaagccacgtcctgattctgtaatgaatttattatatgaaatatacaattcataattatggttttacagttttaatcatttgcatcctgccatatacattaggattatcatatactttagcaataggacgaccatcacttgcaaaatattgaagttgagatttggtattggccgagtaacatccgcccttaatgtatgtgtaagggtcgacactctctgttgttagtacacaatcaacttcgaagtagccttgtgcgtctagccaggaattagcttgagtagtttcatcaatggtagttaacttgggaacagctcttatgccaacatttatagatggcatttgagtatcatgtactgtctcaacatttacctcctcgaatataccaccttgttcaatagggactcgaagatagttctgttcctcatcaaagaatgttgtatctgcgtggggaccctgtatatatttgcgaggtgcattacgtacttgtgttggtgctgtagcttgtgaggaatcggttgttttcgctgccgtcacttcattcttcgttcccgcaggataactaccattataattttgagtaatcaagttgtttggtactggtgcaaactgcggtaccagaggtgcgtagctaaaatcgtaatccatagcaactacatctgtgttgatacatgcacttgcatcgaattcttctataaagtttttgtatggtggaaaccccgccaatatcttagtaccagtagtagcccttgcatcattagtgtaaatggttaaatagtcctgtagatatatctctgcgccagttgcatctgctggtggtttcttagcaaagtctttattatcgttatcgtaaccatacatagcagtcttgagaccatctcgatattggtaggatgtcaatgttgcgaatcccgtaggttgcatgggttcagtatcagaatatgtatactttctgtttgtagaacatataaaaggaatacttctaatacccttagcaacttgcagaaatttgttttgattcagtgtagcattagccgtttgagtatctccagtttgaaatgcaactctagtattccatgttctaatacgtactgaagccgacttagcaaacgtaccaggatagttcttcattcgattatattctgctggactcatataaaagaataagtattcccatggaattgatgccattcctgtcgttagcgcccatcttgctggtctttctccgctggccgcttcagccaatatgacgtttgcatttgctgaagtcagaaacctccatttctttctataagtttgttgaaacttctcaacatgaagtccaattggacgtaatatagctgtagctccgccttccccactacctcctctaaccatgccatccaaattgcctccagttccagggagactcgtccctgacttggataccttactcggcggtccgccaccaccactgcctcgcttcgatccaccgctagaacttgatacttgagacatttctatgtctgccatgtttggttgtggtgcgttacttggtccggcgtctcttaaatcttgtctatctcttacttgttgtaaaaaatcgtctactccgtcttctgattgctgactgggttgttccgcctcctggggtgaccctaaaagatccaaatcaaagttatcaatcgcatcagcctgagttaaaggtaggtcgtcatccccaggaggatcaaagtattccccacgtcttactctagctaggttccattgttccatagcataacgtctttgtccctcattcatactatcccaattaggacgatcctgaggattcggacccaacggtctaactctaggcattgcttaccgtagtatacttttccacttaaatgttcgactgcgtgcttaactcccagcccgattcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtattggatttccaggtccgatattgttcgaatacggtaaaactagtccgttccgtggtggttctccccttgctagattggcagaatctcttctcgcctgtgcttccctttgttcttgctttttcaccttctttagtcccctgcgaggatttactagtctccagtgttctggatatttagccttagtgtccttattccaaggatctccgtacggtttgatcttctcctgtcgctctttcaaccccaaacgtctcaattcccaaggaacggctgcgtagtaatcgtcccgattaccaaatatcgctctcgataaagggttcctagggtcagctgctgctcttcccaattgcccttcgaatattggttgatattggctacctgctccaataaccgcacccccaacacgtggtgtattaggtactctcaatttactgtcacttccggcactggagatactactattagacgagttaataggaatagatacgtatccacccctacgtcgcacccctgtacttgtactgggttgattaaacacactctcttcactactgctgggggtaatagtgctcagctcgatgtactcggacatactgactggaaataacctgcgcatacatatttatatccatgttgatcccccttacttggtgcttgcccataagcccctggcttatcactgacgactgcgacgacgagcagatctatttatacccaatggtgaccttggaaaccatggtataccatgtaaatagtatatttacatggtaaccatggtttccaaggtcaccattgggtataaatagatctgctcgtcgtcgcagtcgtc

SEQ ID NO: 8 - DNA, 5445 bp, Zophobas morio black wasting virus
  misc_feature 1..5445: note = Zophobas morio black wasting virus, strain NY3-molitor
  source 1..5445: mol_type = genomic DNA
agcagatctatttatacccaatggtgaccttggaaaccatggtataccatgtaaattttcaatttactggtataccatggtttccaaggtcaccattgggtataaatagatctgctcgtcgtcgcagtcgtcagtgataagccaggggcttatgggcaagcaccaagtaagggagataaagtgaagtgtataaatagttttgaacatgtaagtagctcattctgttgttgtattccgtgcagtgcagtaatactttggtttggtaagtaggtttaaacataagaagatatatcaccctcagagcttgtatgacattactaagaagaaggtaagagaggtgtacaagaataattggaacgagacgaaggtactaccgaggactatccagagggaactactactagattggttaagatgtgacgagaagatttctgagagtgatgacgatgtggaacgaatagtggcacgtatggaaaggggatgggaggcattgaagccctttggtccgaccacatttgtgtacttgatgagattaccagatgaagtaccgccatttgcacatgaaaggaatcatatcatttgggacttctatttgtggtatgaacaagggcgagaaaagaaaatttgtgaaccgtgttattccgggaagagtcgtttctaccgtccaggatctgctaatgaatggttggaaaaaggatgggtatttaaacgcgtagagaatcactccatgattgatggggacagattgttagaggatttgatatgggatgaagacaactggtgcagtttatgtattgttgagccattatggatccatatatttgatgatgatgattgtttgtttgattatgactatcatttgaagagaaggcgtacatggagtgactcaagctctgaggacagtgatattgattattgtaaacatactgttatgcaagggattcgtataaaccccactttgtataaatttattaatgaataaagtaagtgttatagaattgttgtttcattacagatgaataatgacgaccacttttgggaatattttgacagtactctgggagatgcttccagacgagtgggcgaatcatccgggagtatggtggaagattatggatacgataccactggaccaacagtgcaagaatgcgatgttgcagctaatagggcgttggagcaagaattacaaacaatggtcgaccggttcgttacccgccttgaaaaagaagattggcaagacagcggatactatgtgtcagatgtctttgcctgcgaatcaattggacgagctcaaggattggctaagcgaatggctgaacgagcgggaaatttccgaagaggacttatcctcatatctattcacagcgatcaagatggcggtgcccatatccacaccatccacagctgcgcctacgcaaacaggtcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacgatgaacgaaaacgaggaattgaaccgaaaggtgatggaggtactcgaagtcgcaaaagaagaggtgttggcaacgcaggaggagatggaggaatcagaggggacacaggagtaattcttgatttgattaagaggtatgctgtttgtccattatctgagatagtatatactcgcgagtacttggaaaatcccattgcagttaaaaggttagatgatagggatgtaaagaacgctatagattgccatgctgctattattaatacttggaatagacaggattatgttaagttttatgaggacccaaacactgttaaaatttggagtgcacgtaatgttgactcggtggagttatattatttaactgataatgaatctagagatattattaataaactgcttgattatcaatgtgggttagctaagaaacagtttgtaacggatttgattaatgtaacagacatgaatattcccaagtgtaattgctttctaattgttagtcctccaagtgctggcaagaactttctatgcgatgctattaaagattactatattaatgttggacaaatgcagaaccctaataaatataataccttcgcgtatcaggattgtcacaatagacgacttcttatttggaatgaaccaaactacgaacctagggaaacggagaatttgaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctcctttcttaaaagaggtaataaaaaaaccacgacctgattctgtaatgaatttattatatgaaatatacaattcataattatggttttacagttttaatcatttgcatcctgccatatacattaggattatcatatactttagcaataggacgaccatcacttgcaaaatattgaagttgagatttggtattggccgagtaacatccgcccttaatgtatgtgtaagggtcgacactctctgttgttagtacacaatcaacttcgaagtagccttgtgcgtctagccaggaattagcttgagtagtttcatcaatggtagttaacttgggaacagctcttatgccaatatttatagatggcatttgagtgtcatgtactgtctcaacatttacctcctcgaatataccaccttgttcaatagggactcgaagatagttctgttcctcatcaaagaatgttgtatctgcattgggaccctgtatatatttgcgaggtgcattacgtacttgtgttggtgctgtagcttgtgaggaatcggttgttttcgctgccgtcacttcattctttgttcccgcaggataactaccattatagttttgagtaatcaagttgtttggtactggtgcaaactgcggtaccagaggtgcgtagctaaaatcgtaatccatagcaactacatctgtgttgatacatgcacttgcatcgaattcttctataaagtttttgtatggtggaaaccccgccaatatcttagtaccagtagtagcccttgcatcattagtgtaaatggttaaatagtcctgtagatatatctctgcgccagttgcatctgctggtggtttcttagcaaagtctttattgtcgttatcgtaaccatacatagcagtcttgagaccatctcgatattggtaggatgtcaatgttgcgaatcccgtaggttgcatgggttcagtatcagaatatgtatactttctgtttgtagaacatataaaaggaatacttctaatacccttagcaacttgcagaaatttgttttgattcagtgtagcattagctgtttgagtatctccagtttggaatgcaactcttgtgttccacgttctaatacgtactgaagcggacttagcgaacgtaccaggatagttcttcattctgttatattctgctggactcatatagaagaataagtattcccatggaattgatgccattcctgtagttaatgcccatcttgctggtctttctccgctagctgcctcggccaatatgacgtttgcatttgctgaagtcagaaacctccatttctttcggtatgtttgttggaatttttcgacgtgaagtccaataggacgtaatatagctgtagctccgccttccccaccacctcctctaaccatgccatctaagttgcctccagttccagggagactcgtccctgacttggataccttactcggcggtccgccaccaccactacctcgcttcgatccaccgctagaacttgatacttgagacatttctatgtccgccatgtttggttgtggtgcgttacttggtccggcgtctcttaaatcttgtctatctcttacttgttgtaaaaaatcgtctactccgtcttctgattgctgactgggttgttccgcctcctggggtgaccctaaaagatccaaatcaaagttatcaatcgcatcagcctgagttaaaggtaggtcgtcatccccaggaggatcaaagtattccccacgtcttactctagctaggttccattgttccatagcataacgtctttgtccctcattcatactatcccaattaggacgatcctgaggattcggacccaacggtctaactctaggcatctcttaccgtagtatacttttccgcttaaatgttcgactgcgtgcttaactcccagcccgattcctcccactgccgcgtgtaccctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtattggatttccaggtccgatattgttcgaatacggtaaaactagtccgttccgtggtggttctccccttgctagattggcagaatctcttctcgcctgtgcttccctttcttcttgcttcttcaccttctttagtcccctgcgaggatttactagtctccagtgttctggatatttagccttagtgtccttattccaaggatctccgtacggtttgatcttctcctgtcgctctttcaaccccaaacgtctcaattcccaaggaacggctctgtagtaatcgtctcgattaccaaatattgctctcgataaagggttgttagggtcagctgctgctcttcccaattgcccttcgaatattggttgatattggctacctgctccaaaagccgcacccccaacacgtggtgtattaggtactctcaatttactgtcacttccggcactggagatactactattagacgagttaataggaatagatacgtatccacccctacgtcgcacccctgtacttgtactgggttgattaaacacactctcttcactactgctgggggtaatagtgctcagctcgatgtactcggacatactgactggaaatgacctgcgcatacatatttatatccatgttgatcccccttacttggtgcttgcccataagcccctggcttatcactgacgactgcgacgacgagcagatctatttatacccaatggtgaccttggaaaccatggtataccatgtaaattttctatttacaggtataccatggtttccaaggtcaccattgggtataaatagatctg

SEQ ID NO: 9 - DNA, 5406 bp, Zophobas morio black wasting virus
  misc_feature 1..5406: note = Zophobas morio black wasting virus, strain PA-molitor
  source 1..5406: mol_type = genomic DNA
agcagatctatttatacccaatggtgaccttggaaaccggttaccatgtaaattttctatttacggtataccatggtttccaaggtcaccattgggtataaatagatctgctcgtcgtcgcagtcgtcagtgataagccaggggcttatgggcaagcaccaagtaagggagataaagtgaagtgtataaatagtgttcaacatgtaagtagctcattctgttgttgtatttcatgcagtgcagtaatactttgcattggtaagtaggtttaaacataagaagatatatcaccctcagagcttgtatgacattactaagaagaaggtaagagaggtgtacaagaataattggaacgagacgaaggtgctaccgaagactatccagagggaactactactagattggttaagatgtgacgagaagatttctgagagtgacgacgatgtggaacgaatagtggcacgtatggaaaggggatgggaggcattgaagccctttggtccgaccacatttgtgtacttgatgagattaccagatgaagtaccgccatttgcacatgaaaggaatcatatcatttgggacttctatttgtggtatgaacaagggcgagaaaagaaaatttgtgaaccgtgttattccgggaagagtcgtttctaccgtccaggatctgctaatgaatggttggaaaaaggatgggtatttaaacgcgtagagaatcactccatgattgatggggacagattgttagaggatttgatatgggatgaagacaactggtgcagtttatgtattgttgagccattatggatccatatacttgatgatgatgattgtttgtttgattatgactatcatttcaagagaagacgtacatggagtgactcaagctctgaggacagtgatattgattattgtaaacatactgttatgcaagggattcgtataaaccccactttgtataaatttattaatgaataaagcaagtgttatagaattgttgtttcattacagatgaataatgacgaccacttttgggaatattttgacagtactctgggagatgcttccggacgagtgggcgaatcatccaggagtatggtggaagattatggatacgataccactggaccaacaatgcaagaatgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgaagaggacttatcctcatatctattcacagcgatcaagatggcggtgcccatatccacaccatccacagctgcgcctacgcaaacaggtcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacgatgaacgaaaacgaggaattgaaccgaaaggtgatggaggtactcgaagtcgcaaaagaagaggtgttggcaacgcaggaggagatggaggaatcagaggggacacaggagtaattcttgatttaattaagaggtatgctgtttgtccattatctgagatagtatatactcgcgagtacttggaaaatcccattgcagttaaaaggttagatgatagggatgtaaagaacgctatagattgccatgctgctattattaatacttggaatagacaggattatgttaagttttatgaggacccaaacactgttaaaatttggagtgcacgtaatgttgactcggtggagttatattatttaactgataatgaatctagagatattattaataaactgcttgattatcaatgtgggttagctaagaaacagtttgtaacggatttgattaatgtaacagacatgaatattcccaagtgtaattgctttctaattgttagccctccaagtgctggcaagaactttctatgcgatgctattaaagattactatattaatgttggacaaatgcagaaccctaataaatataataccttcgcgtatcaggattgtcacaatagacgacttcttatttggaatgaacccaactacgaacctagggaaacggagaatttgaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctcctttcttaaaagaggtaataaaaaaaccacgacctgattctgtaatgaatttattatatgaaatatacaattcataattatggtttcacagttttaatcatttgcatcctgccatatacattaggattatcatatactttagcaataggacgaccatcacttgcaaaatattgaagttgagatttggtattggccgagtaacatccgcccttaatgtatgtgtaagggtcgacactctctgttgttagtacacaatccacttcgaagtagccttgtgcgtctagccaggaattagcttgagtagtttcatcaatggtagttaacttgggaacagctcttatgccaatatttatagatggcatttgagtgtcatgtactgtttcaacatttacctcctcgaatataccaccttgttcaatagggactcgaagatagttctgttcctcatcgaagaatgttgtatctgcgtggggaccctgtatatatttgcgaggtgcattacgtacttgtgttggtgctgtagcttgtgaggaatcggttgttttcgctgccgtcacttcattctttgttcccgcaggataactaccattataattttgagtaatcaagttgtttggtactggtgcaaactgtggtaccagaggtgcgtagctaaaatcgtaatccatagcaactacatctgtgttgatacatgcacttgcatcgaattcttctataaagtttttgtatggtggaaaccccgccaatatcttagtaccagtagtagcccttgcatcattagtgtaaatggttaaatagtcctgtagatatatctctgcgccagttgcatctgctggtggtttcttagcaaagtctgcactgtcattatcgtaaccatacatagcaatcttgagaccatctcgatattggtaggatgtcaaggttgcgaatccagtaggttgcatgggttcagtatcagaatatgtatactttctgtttgtagagcatataaaaggaatacttctaatccccttagccacttgtaggaatttattctgatttagtgtagcattagatgtttgagtatctccagtttggaatgcaactcttgtgttccacgtcctaatacgtactgaagcggacttagcaaacgtaccaggatagttcttcattctgttatattctgctggactcatataaaagaataagtattcccatggaattgatgccattcctgtagttaatgcccatcttgctggtctttctccgctggccgcttcagccaatatgacgtttgcatttgctgaagtcagaaacctccatttctttctataagtttgttgaaacttctcaacatgaagtccaattggacgtaatatagctgtagctccgccttccccactacctcctctaaccataccatccaaattgcctcctgttcctggtaaacttgtcccagacttggataccttactcggcggtccgctaccatcactacctcgcttggatccactactagagctagatacagacatttctacgtccgccatgtttggttgtggtgcgttacttggtccggcgtctcttaaatcttgtctatctcttacttgctgtaaaaaatcgtctactccgtcttctgattgctgactgggttgttccgcctcctggggtgaccctaaaagatccaaatcaaagttatcaatcgcatcagcctgagttaaaggtaggtcgtcatccccaggaggatcaaagtattccccacgtcttactctagctaggttccattgttccatagcataacgtctttgtccctcattcatactatcccaattaggacgatcctgaggattcggacccaacggtctaactctaggcattgcttaccgtagtatacttttccgcttaaatgttcgactgcgtgcttaactcccagcccgattcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtattggatttccaggtccgatattgttcgaaaacggtaaaactagtccgttccgtggtggttctccccttgctagattggcagaatctcttctcgcctgtgcttccctttgttcttgcttcttcgccttctttagtcccctgcgaggatttactagtctccagtgttctggatatttagccttagtgtccttattccaaggatctccgtacggtttgatcttctcctgtcgctctttcaaccccaaacgtctcaattcccaaggaacggctgcgtagtaatcgtctcgattaccaaatattgctctcgataaagggttcctagggtcagctgctgctcttcccaattgcccttcgaatattggttgatattggctacctgctccaataaccgcacccccaacacgtggtgtattaggtactctcaatttactgtcacttccggcactggagatactactattagacgagttaataggaatagatacgtatccacccctacgtcgcacccctgtacttgtactgggttgattaaacacactctcttcactactgctgggggtaatagtgctcagctcgatgtactcggacatactgactggaaatgacctgcgcatacatatttatatccatgttgatcccccttacttggtgcttgcccataagcccctggcttatcactgacgactgcgacgacgagcagatctatttatacccaatggtgaccttggaaaccggttaccatgtaaattttctatttacggtataccatggtttcca

SEQ ID NO: 10 - DNA, 5449 bp, synthetic construct
  misc_feature 1..5449: note = Synthetic: sequence of engineered strain 1
  source 1..5449: mol_type = other DNA
gacgaccagatctatttatacccaatggtgaccttggaaacctggttaccatgtaaattttctatttactggtataccatggtttccaaggtcaccattgggtataaatagatctggtcgtcgtcgcagtcgtcagtgataagccaggggcttatgggcaagcaccaagtaagggggataaagtgaagtgtataaatagtgttcaacatgtaagtagctcattctgttgttgtatttcgtgcagtgcagtaatactttggtttggtaagtaggtttaaacataagaagatatatcaccctcagagcttgtatgacattactaagaagaaggtaagagaggtgtacaagaataattggaacttgctggtactaccgaatcgtatccagagggaactactactagattggttaagatgtgacgagacaattccggagagtgatgacgatgtggaacgaatagtggcacgtatggaaaggggatgggaggcattgaagccctttggttcgactacatttgtgtacttgatgagattaccagatgaagtaccgccatttgcacatgaaaggaatcatatcatttgggacttttacttgtggtatgaacaagggcgagaaaagaaaatttgtgaaccgtgttattccgggaagagtcgtttctactgtccaggatctgctaatgaatggttagaaaaaggatgggtatttaaacgcgtagagaatcactccatgattgatggggacagattgttacaggagttgatatgggatgaagacaactggtgcagtttatgtcttgttgagccattatggatccatatattggatgatgatgattgtttgtttgattatgactatcatttgaagagaaggcgtacatggagtgactcaagctctgaggacagtgatattgattattgtaaacatactgttatgcaagggattcgtatgaaccccactttgtataaatttattaatgaataaagcaagtgttatagaattgttgtttcattacagatgaataatgacgaccacttttgggaatattttgacagtactctgggagatgcttccggacgagtgggcgaatcatccgggagtatggtggaagattatggatacgataccactggaccaacaatgcaagaatgcgatgttacagctaatagggcgttggagcaagaattacaaacaatggtcgaccggttcgttacccgccttgaaaaagaagattggcaagacagcggatactatgtgtcagatgtctttgcctgcgaatcaattggacgagctcagggattggctaagcgaatggctgaacgagcgggaaatttccgacgaggacttatccttatatctatccacagcgatcaggatggcagtgcccatgtccacaccatccacagctgcgcctacgcaaacagggcgtgcaggtgctacttcaaagcgttccccgaagcgcaagaggacgctagacgacttcttcgaaagcctccacccatcgaaacgttcaagcgaagcgattgggaaaatatcacaaagtatttttgtacgaaagggagacgagcaacgttcgctaaaatcaacggtgccatacaaagacttcctcttgaaattacaaatctatccgacattatcctatcaggccaagttgaaggaggatcacactcaggcctggagaactgccacgacccgcttgacaataacaatgaacgaaaacgaggaattgaaccgaaaggtgatggaggtactcgaagtcgcaaaagaagaggtgttggcaacgcaggaggagatggaggaatcagaggggacacaggagtaattcttgatttaattaagaggtatgctgtttgtccattatctgagatagtatatactcgcgagtacttggaaaatcgtattgcagttaaaaggttagatgatagggatgtaaagaacgctatagattgccatgctgctattattaatacttggaatagagaggattatgttaagttctatgaggacccaaacactgttaaaatttggagtgcacgtagtattgacttggtggaattatattatttaactgataatgaatcgagggatattattaataaactgctggattaccaatgtgggttagctaagaaacagtttgtaacagatttgattaatgtaacagacatgaatattcccaagtgtaattgctttctaattgttagccctccaagtgctggcaagaactttctatgcgatgctattaaagattactatcttaatgttggacaaatgcagaaccctaataaatataataccttcgcgtatcaggattgtcacaatagacgacttcttatttggaatgaaccaaactacgaacctagagaaacggagaatttaaagatgttatttggaggggataacttgtctgctaatgttaagtgtaaacctcaagctaatgtaaagcgtactccagtaatatgtatgagtaatgttgtacccagatttgctaatcatgaagcatttgctgacagagttattacatattattggaatgctgctccgttcttgaaagaggtaataaaaaaaccacgacctgattctgtaatgaatttattatatgaaatatacaattcataattatggttttacagttttaatcatttgcatcctgccatatacattaggattatcatatactttagcaataggacgaccatcatttgcaaaatattgaagttgagatttggtattggccgagtaacatccgcccttaatgtatgtgtaagggtcgacactctctgttgttagtacacaatcaacttcgaagtagccttgtgcgtctagccaggaattagcttgagtagtttcatcaatggtagttaacttgggaacagctcttatgccaacatttatagatggcatttgagtgtcatgtactgtctcaacatttacctcctcgaatatacctccttgttcaatagggactcgaagatagttctgttcctcatcaaagaatgttgtatctgcattgggaccctgtatatatttgcgaggtgcattacgtacttgtgttggtggtgtagcttgtgaggaatcggttgttttcgctgccgtcacttcattctttgttcccgcaggataactagcattatagttttgagtgatcaagttgtttggtactggtgcaaactgtggtaccagaggtgcgtagctaaaatcgtaatccatggcaactacatctgtgttgatacatgcacttgcatcgaattcttctataaagtttttgtatggtggaaaccccgccaatatcttagtaccagtagtagcccttgcatcattagtgtaaatggttaaatagtcctgtagatatatctctgcgccagttgcatctgctggtggtttcttagcaaagtctggactgtcattatcgtaaccatacatagcaatcttgagaccatctcgatattcgtaggatgtcaatgttgcgaatcccgtaggttgcatgggttcagtatcagaatatgtatactttctgtttgtagagcatataaaaggaatacttctaatacccttagcaacttgcagaaatttgttttgattcagtgtagcattagccgtttgagtatctccagtttgaaatgcaactctagtattccatgttctaatacgtactgaagccgacttagcgaacgtcccaggatagttcttcattcgattatattctgctggactcatataaaagaataagtattcccatggaattgatgccattcctgtggttaatgcccatcttgctggtctttctccgctagctgcctcggccaagatgacgttggcatttgctgaagttaggaatctccatttctttctataagtttgttgaaacttttcaacatgaagtccaattggacgtaatatagctgtagctccgccttccccactacctcctctagccataccatccaaattgcctcctgttcctggtaaacttgtcccagacttggataccttactcggcggtccgctaccatcactacctcgcttggatccactactagagctagatacttgagccatttctacgtccgccatgtttggttgtggtgcgttacttggtccggcgtctcttaaatcttgtctatctcttacttgttgtaaaaaatcgtctactccgtcttctgattgctgactgggttgttccgcctcctggggtgaccctaaaagatccaaatcaaagttatcaattgcatcagcctgagttaaaggtaggtcgtcatccccaggaggatcaaaatattctccacgtcttactctagctaggttccattgttccatagcatagcgtctttgtccatcattcatactatcccaattaggacgatcttggggattcggacccagcggtctaactctaggcattgcttaccgtaatatacttttccacttagacgttcgactgcgtgcttaactcccaacccgactcctcccactgccgcgtgtatcctagatataggatcctgaccctggattgcctcgtgtgcgaattgtgagattgcttctctatccgctgacagcacgtcgctatcgctcttagcttgttgataatgtaagtcgtgaccctgcgcaataaggtcagctctgttggtcgcgggacgtactggatttccaggtccgatattgttcgaaaacggtaaaactagtccgttccgtggtggttctccccttgctagattggcagaatctcttctcgcctgcgcttccctttgttcttgcttcttcgccttgtttagtcccctgcgaggatttactagtctccagtgttctggatatttagccttagtgtccttattccaaggatctccgtacggtttgatcttctcctgtcgctctttcaaccccaaacgtctcaattcccaaggaacggctgcgtagtaatcgtctcgattaccaaatattgctctcgataaagggttgttagggtcagctgctgctcttcccaattgcccttcgaatattggttgatattggctacctgctccaataaccgcacccccaacacgtggtgtattaggtaccttcaatttactgtcacttccggcactggagatactactattagacgagttaataggaatagatacgtatccacccctacgtcgcacccctgtacttgtattgggttgattaaacacactctcttcactactgctgggggtaatagtgctcagctcgatgtactcggacatactgactggaaataacctgcgcatacatatttatatccatgttgatcccccttacttggtgcttgcccataagcccctggcttatcactgacgactgcgacgacgaccagatctatttatacccaatggtgaccttggaaaccggttaccatgtaaattttctatttacggtataccatggtttccaaggtcaccattgggtataaatagatctggtcgtcgtc

SEQ ID NO: 11 - DNA, 21 bp, synthetic construct
  misc_feature 1..21: note = Synthetic
  source 1..21: mol_type = other DNA
gacagcggatactatgtgtca

SEQ ID NO: 12 - DNA, 21 bp, synthetic construct
  misc_feature 1..21: note = Synthetic
  source 1..21: mol_type = other DNA
aatttcaagaggaagtctttg